(19) United States
(12) Patent Application Publication
(10) Pub. No.: US 2026/0293910 A1
(43) Pub. Date: Oct. 1, 2026

(54) ENDOPHYTE COMPOSITIONS AND METHODS FOR IMPROVED PLANT HEALTH

(71) Applicant: INDIGO AG, INC., Charlestown, MA (US)

(72) Inventors: David A. HUBERT, Durham, NC (US); Shib Sankar BASU, Apex, NC (US); John Paul SMITH, Dayton, KY (US); Cesar U. VALENCIA, Durham, NC (US); Steven E. SCREEN, Wake Forest, NC (US); Carlos VASSALLO, Buenos Aires (AR)

(21) Appl. No.: 18/993,287

(22) PCT Filed: Jul. 13, 2023
(86) PCT No.: PCT/US2023/070181

§ 371 Date: Jan. 10, 2025

Related U.S. Application Data
(60) Provisional application No. 63/388,891, filed on Jul. 13, 2022.

(51) Int. Cl. A01N 63/22; A01N 25/04; A01N 37/22; A01N 43/36; A01N 43/56; A01P 3/00; A01P 21/00; C12N 1/20; C12R 1/07

(52) U.S. Cl. CPC A01N 63/22; A01N 25/04; A01N 37/22; A01N 43/36; A01N 43/56; A01P 3/00; A01P 21/00; C12N 1/20; C12R 2001/07

(57) ABSTRACT

This invention relates to compositions and methods for improving plant health, wherein a plant is heterologously disposed to one or more endophytes, or derived from a plant element heterologously disposed to one or more endophytes.

### CROSS REFERENCE TO RELATED APPLICATIONS

This application is a U.S. National Phase Application under 35 U.S.C. § 371 of International Application No. PCT/US2023/070181 filed Jul. 13, 2023, which claims the benefit of U.S. Provisional Application No. 63/388,891, filed Jul. 13, 2022, entitled ENDOPHYTE COMPOSITIONS AND METHODS FOR IMPROVED PLANT HEALTH, which are incorporated by reference in their entirety.

### SEQUENCE LISTING

The instant application contains a Sequence Listing which has been submitted electronically in XML format and is hereby incorporated by reference in its entirety. Said XML copy, created on May 15, 2026, is named 10248PCTUS1_SL_v2.xml and is 183,609 bytes in size.

### BACKGROUND

According to the United Nations Food and Agriculture Organization, the world's population will exceed 9.6 billion people by the year 2050, which will require significant improvements in agriculture to meet growing food demands. There is a need for improved methods and agricultural plants that will enable a near doubling of food production with fewer resources and more environmentally sustainable inputs, and for plants with improved responses to various stresses.

### BRIEF DESCRIPTION OF THE DRAWINGS

FIG. 1A shows results of 2022 USA field trials in soybean fields across 7 trials in fields inoculated with Fusarium graminearum. Plant height was measured 17 full days after full emergence and compared to untreated controls. The treatment “MIC-54924+Chem” was endophyte MIC-54924 plus sedaxane, mefenoxam, and fludioxonil. The treatment “Chemical Control” was only sedaxane, mefenoxam, and fludioxonil. Untreated controls had an average plant height of 15.46 cm at 17 full days after full emergence.

FIG. 1B shows results of 2022 USA field trials in soybean fields across 7 trials in fields inoculated with Fusarium graminearum. Yield was measured and compared to untreated controls. The treatment “MIC-54924+Chem” was endophyte MIC-54924 plus sedaxane, mefenoxam, and fludioxonil. The treatment “Chemical Control” was only sedaxane, mefenoxam, and fludioxonil. Untreated controls had an average yield of 67.56 bu/acre.

FIG. 2 shows results of USA soybean field trials where Pythium ultimum inoculum was applied in-furrow. Root fresh weight was measured 17 full days after full emergence and compared to untreated controls. Twelve MIC-54924 (“MIC-54924”) treated plants, twelve plants treated with a commercial chemical nematicide (“Chemical Control”), and twelve plants treated with a reference biological biotic product (“Biological Benchmark”) were measured. Error bars represent 95% confidence intervals. Untreated controls had an average fresh root weight at 17 full days after full emergence of 1.83 g/plant. Treatment with MIC-54924 resulted in a highly significant (p<0.01) increase in root weight compared to untreated controls with a 100% win rate.

FIG. 3 shows results from 8 replicated soybean field trials in Argentina. Trials were under natural infestation of plant pathogens. Trials included three treatments of MIC-54924 applied to soybeans at three dosages 0.5 mL/kg seeds, 1.3 mL/kg seeds, 2 mL/kg seeds, as well as treated with a commercial chemical fungicide (“Chemical Control”). All treatments showed a positive average yield delta relative to untreated controls, with two dosages of MIC-54924 showing greater increases in yield than the Chemical Control.

FIG. 4 shows exemplary images of a plate based anti-biosis assay in which a fungal pathogen Cercospora sojina was introduced in the center of each plate surrounded by treated or untreated seeds. The image labeled “MIC-54924” shows 12 representative plates in which MIC-54924 treated seeds surround the central position in which Cercospora sojina was introduced. The image labeled “Chemical Control” shows 4 representative plates in which seeds treated with commercial chemical fungicide surround the central position in which Cercospora sojina was introduced. The image labeled “Untreated” shows 4 representative plates in which untreated seeds surround the central position in which Cercospora sojina was introduced. The line adjacent to the label “A” shows an example of the diameter of the pathogen culture on a plate with MIC-54924 treated seeds. The line adjacent to the label “B” shows an example of the diameter of the pathogen culture on a plate with Chemical Control treated seeds. The line adjacent to the label “C” shows an example of the diameter of the pathogen culture on a plate with untreated seeds.

FIG. 5 shows the emergence (plants/acre) of soybean grown in field conditions under infestation with Rhizoctonia solani. Emergence was measured at 10 days after planting. Results labeled “MIC-54924” show emergence of soybean seeds treated with MIC-54924 alone. Results labeled “MIC-54924 with Fludioxonil Sedaxane Mefenoxam” show emergence of soybean seeds treated with MIC-54924 and Fludioxonil, Sedaxane, and Mefenoxam. Results labeled “Fludioxonil Sedaxane Mefenoxam” show emergence of soybean seeds treated with only Fludioxonil, Sedaxane, and Mefenoxam. While all treatments outperformed untreated plants (labeled “Stress Control”), the combination of MIC-54924 with chemical fungicides resulted in improved results relative to chemical fungicides alone.

FIG. 6 shows exemplary results from 7 replicated greenhouse experiments in winter wheat plants infected with Pythium ultimum. Shoot fresh weight (g) of plants treated with MIC-54924 (labeled “MIC-54924”) consistently resulted in higher average shoot fresh weight compared to untreated plants “Stress Control”, 100% win rate. Error bars are +/−one standard deviation.

FIG. 7 shows exemplary results from 16 replicated greenhouse experiments in winter wheat plants infected with Fusarium oxysporum. Shoot fresh weight (g) of plants treated with MIC-54924 (labeled “MIC-54924”) consistently resulted in higher average shoot fresh weight compared to untreated plants “Stress Control”, 75% win rate. Error bars are +/−one standard deviation.

FIG. 8 shows exemplary images of a plate based anti-biosis assay in which a fungal pathogen Pythium ultimum was introduced in the center of each plate. Positions labeled “A” show where MIC-54924 was placed on the plates. Positions labeled “B” show where Mefenoxam was placed on the plate. The top two plates show pathogen growth on King's medium B agar. The top two plates show pathogen growth on King's medium B agar without the carbohydrate source glycerol. MIC-54924 shows a greater zone of inhibition on the agar without the carbohydrate source glycerol.

FIG. 9 shows exemplary images of a plate based anti-biosis assay in which a fungal pathogen Fusarium graminearum was introduced in the center of each plate. Positions labeled “A” show where MIC-54924 was placed on the plates. Positions labeled “B” show where Mefenoxam was placed on the plate. The top two plates show pathogen growth on King's medium B agar. The top two plates show pathogen growth on King's medium B agar without the carbohydrate source glycerol. MIC-54924 shows a greater zone of inhibition on the agar without the carbohydrate source glycerol.

FIG. 10 shows exemplary images of a plate based anti-biosis assay in which a fungal pathogen Rhizoctonia solani was introduced in the center of each plate. Positions labeled “A” show where MIC-54924 was placed on the plates. Positions labeled “B” show where Mefenoxam was placed on the plate. The top two plates show pathogen growth on Yeast Extract Peptone Dextrose Agar. The top two plates show pathogen growth on Yeast Extract Peptone Dextrose Agar without the carbohydrate source dextrose. MIC-54924 shows a greater zone of inhibition on the agar without the carbohydrate source dextrose.

FIG. 11 shows a comparison of predicted structures MIC-54924 W1-99 and Bacillus amyloliquefaciens W1, the area of the MIC-54924 W1-99 structure labeled “A” shows a unique feature of MIC-54924 W1-99.

FIG. 12 shows a comparison of predicted structures MIC-54924 W1-98 and Bacillus amyloliquefaciens W1, the area of the MIC-54924 W1-98 structure labeled “A” shows a unique feature of MIC-54924 W1-98.

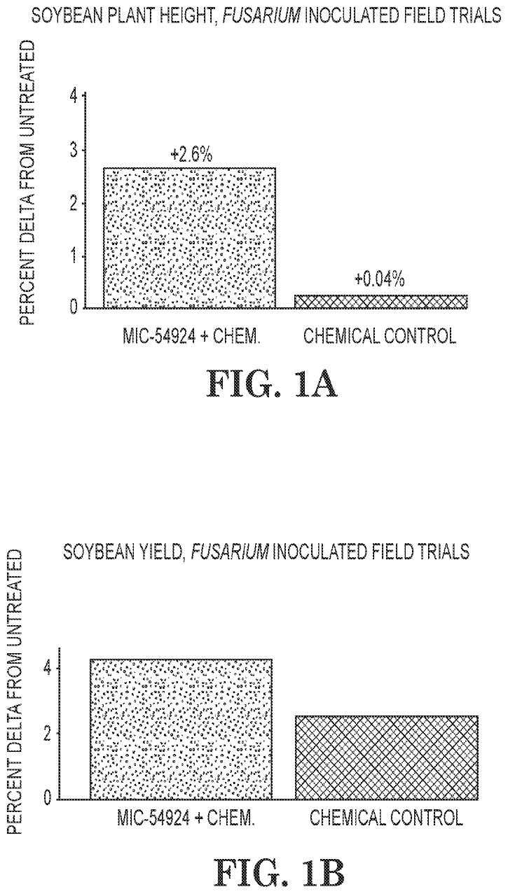

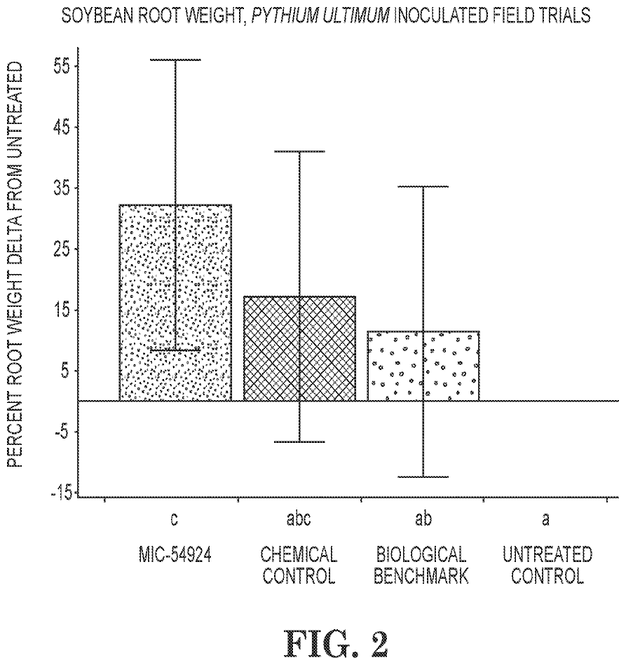

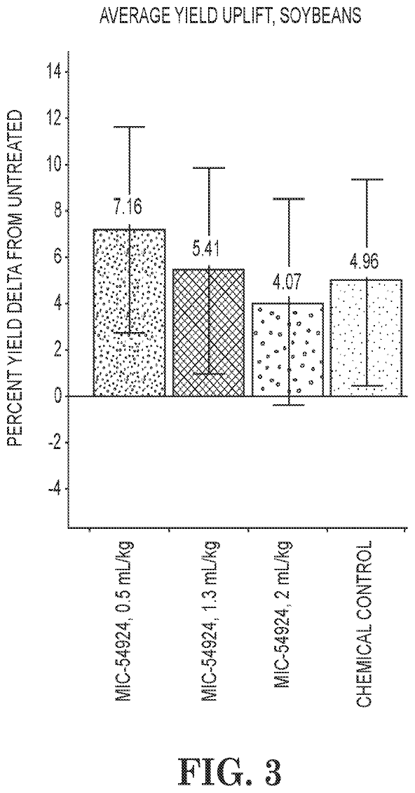

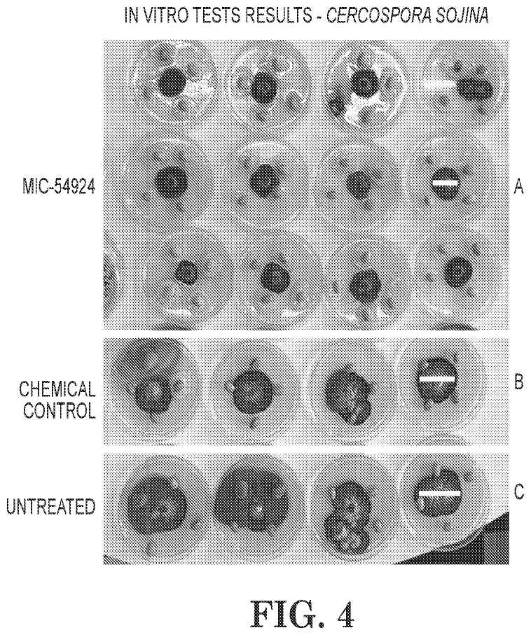

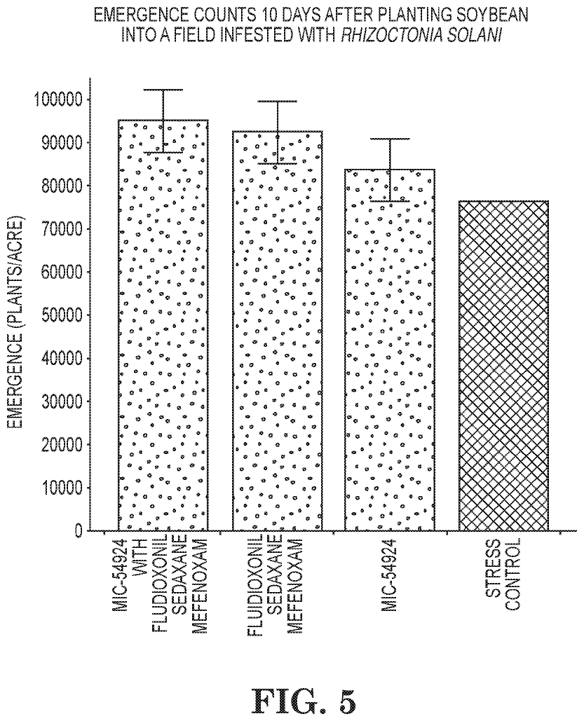

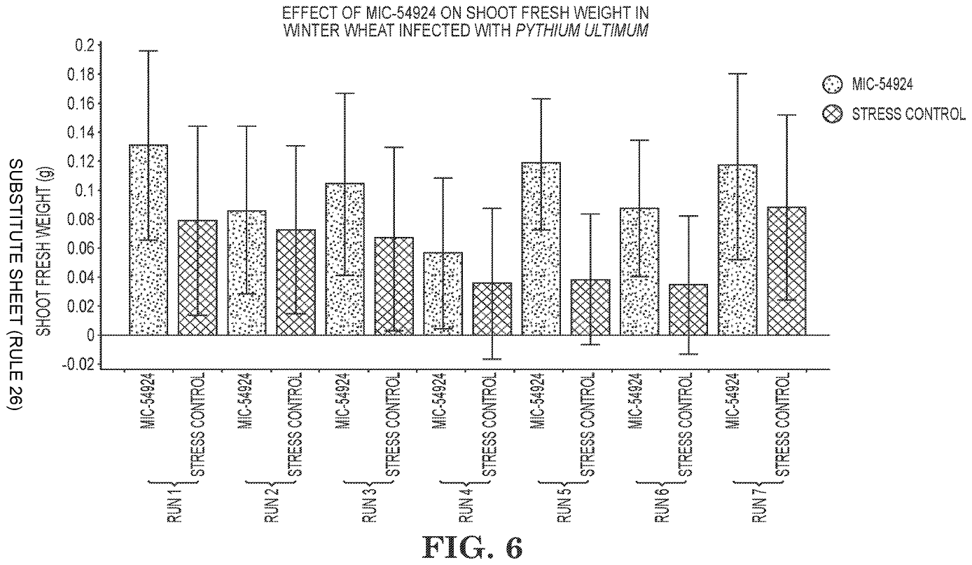

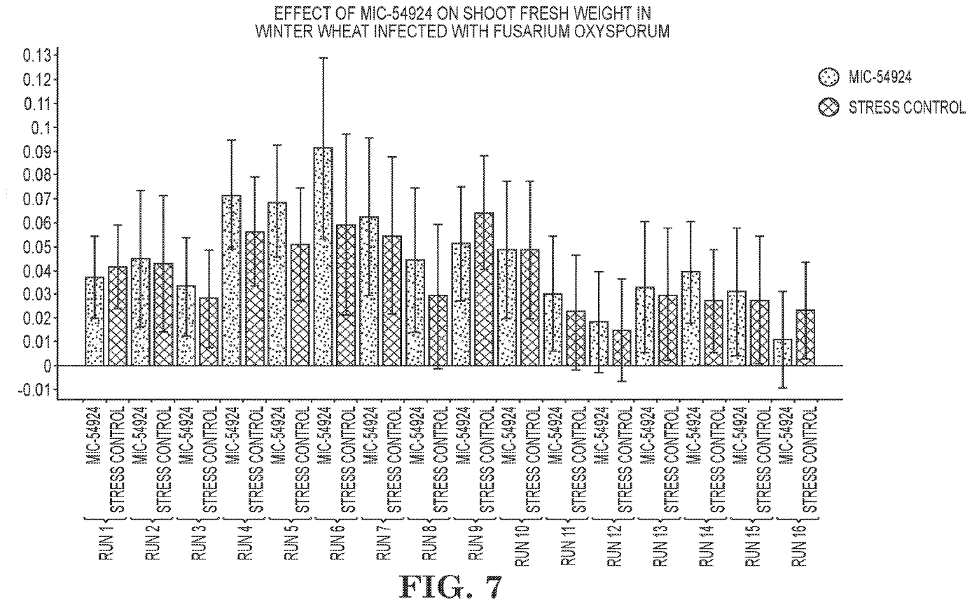

### SUMMARY OF INVENTION

Disclosed herein are methods of improving plant health, comprising heterologously disposing one or more endophytes to a plant element in an effective amount to improve a trait of agronomic importance in a plant derived from the treated plant element relative to a reference plant derived from a reference plant element, wherein the one or more endophytes comprise at least one polynucleotide sequence that is at least 97% identical to SEQ ID NO. 5-51, or one or more genes encoding a protein whose amino acid sequence is selected from the group consisting of SEQ ID NOs. 52-134, or combinations thereof.

In some embodiments, the method additionally comprises the step of placing the plant element in or on a growth medium. In some embodiments, the one or more endophytes are heterologously disposed to a plant element prior to placing the treated plant element in or on a growth medium. In some embodiments, the one or more endophytes are heterologously disposed to a plant element after placing the plant elements in or on a growth medium. In some embodiments, the one or more endophytes are heterologously disposed to a plant element concurrently with placing the plant elements in or on a growth medium.

In some embodiments, the one or more endophytes are heterologously disposed to a plant element at least two times. In some embodiments, the one or more endophytes are heterologously disposed to a plant element via a seed treatment or soil pre-treatment and one or more foliar applications. In some embodiments, the one or more endophytes are heterologously disposed to a plant element via a seed treatment or soil pre-treatment and one or more floral applications. In some embodiments, the one or more endophytes are heterologously disposed to a plant element via one or more seed treatments or soil pre-treatments, one or more foliar applications, and one or more floral applications. In some embodiments, the one or more endophytes are heterologously disposed to a plant element via seed treatment, root wash, seedling soak, foliar application, floral application, soil inoculum, in-furrow application, sidedress application, soil pre-treatment, wound inoculation, drip tape irrigation, vector-mediation inoculation, injection, osmopriming, hydroponics, aquaponics, aeroponics, or combinations thereof.

In some embodiments, the one or more endophytes are heterologously disposed to a plant element of a different plant variety from the variety of the plant element from which the one or more endophytes were obtained. In some embodiments, the one or more endophytes are heterologously disposed to a plant element of the same plant variety as the variety of the plant element from which the one or more endophytes were obtained. In some embodiments, the one or more endophytes are heterologously disposed to a plant element of a different plant species from the species of the plant element from which the one or more endophytes were obtained. In some embodiments, the one or more endophytes are heterologously disposed to a plant element of the same plant species as the species of the plant element from which the one or more endophytes were obtained.

In some embodiments, the plant elements are allowed to germinate. In some embodiments, the plant elements are grown to yield.

In another aspect, disclosed herein are synthetic compositions, comprising one or more endophytes heterologously disposed to a treatment formulation, wherein the one or more endophytes comprise at least one polynucleotide sequence that is at least 97% identical to SEQ ID NO. 5. In some embodiments, the composition additionally comprises a plant element. In some embodiments, the one or more endophytes are capable of improving a trait of agronomic importance in a plant derived from the plant element (for example, when grown from a treated seed) relative to a plant derived from a reference plant element.

In some embodiments, the synthetic composition additionally comprises one or more of a surfactant, a buffer, a tackifier, a microbial stabilizer, a fungicide, an anticomplex agent, an herbicide, a nematicide, an insecticide, a plant growth regulator, a rodenticide, a desiccant, a nutrient, an excipient, a wetting agent, a salt, and a polymer. In some embodiments, the polymer is a biodegradable polymer selected from the group consisting of alginate, agarose, agar, gelatin, polyacrylamide, chitosan, polyvinyl alcohol, and combinations thereof. In some embodiments, the biodegradable polymer is alginate, and the alginate is sodium alginate.

In some embodiments, the synthetic composition comprises one or more endophytes of the present invention and one or more chemical or biological agent capable of killing, impeding the feeding and or growth and or reproduction of, repelling, and or reducing the severity or extent of infection to a plant host of, a pest of a plant, including without limitation chemical or biological agents that are acetylcholinesterase (AChE) inhibitors, GABA-gated chloride channel blockers, sodium channel modulators, nicotinic acetylcholine receptor (nAChR) competitive modulators, nicotinic acetylcholine receptor (nAChR) allosteric modulators—Site I, Glutamate-gated chloride channel (GluCl) allosteric modulators, Chordotonal organ TRPV channel modulators, Nicotinic acetylcholine receptor (nAChR) channel blockers, Octopamine receptor agonists, Voltage-dependent sodium channel blockers, multi-site inhibitors, Ryanodine receptor modulators, chordotonal organ modulators (wherein the chordotonal organ modulator does not bind to the Nan-lav TRPV channel complex), GABA-gated chloride channel allosteric modulators, GABA-gated chloride channel allosteric modulators-Site II, nicotinic acetylcholine receptor (nAChR) Allosteric Modulators-Site II, Juvenile hormone mimics, Mite growth inhibitors affecting CHS1, Inhibitors of chitin biosynthesis affecting CHS1, Inhibitors of chitin biosynthesis-type 1, Molting disruptors-Dipteran, Ecdysone receptor agonists, Inhibitors of acetyl COA carboxylase, Inhibitors of mitochondrial ATP synthase, Uncouplers of oxidative phosphorylation via disruption of the proton gradient, Mitochondrial complex III electron transport inhibitors, Mitochondrial complex I electron transport inhibitors, Mitochondrial complex IV electron transport inhibitors, Mitochondrial complex II electron transport inhibitors, Microbial disruptors of insect midgut membranes, Host-specific occluded pathogenic viruses, other active compounds (such as Azadirachtin, Benzoximate, Bromopropylate, Chinomethionat, Dicofol, Lime sulfur, Mancozeb, Pyridalyl, Sulfur, Chlorantraniliprole, Clothianidin, Tioxazafen, Fluopyram), other active bacterial agents (such as certain Burkholderia strains including without limitation Burkholderia rinojenses, Wolbachia pipientis), other active fungal agents (such as Beauveria bassiana strains, Metarhizium anisopliae strain F52, Paecilomyces fumosoroseus Apopka strain 97), biological essence including synthetics or extracts or refined or unrefined oils (such as Dysphania ambrosioides near ambrosioides extract, fatty acid monoesters with glycerol or propanediol, neem oil), non-specific mechanical disruptors (such as Diatomaceous earth), or combinations thereof. Examples of AChE inhibitors include without limitation Carbamates (such as Alanycarb, Aldicarb, Bendiocarb, Benfuracarb, Butocarboxim, Butoxycarboxim, Carbaryl, Carbofuran, Carbosulfan, Ethiofencarb, Fenobucarb, Formetanate, Furathiocarb, Isoprocarb, Methiocarb, Methomyl, Metolcarb, Oxamyl, Pirimicarb, Propoxur, Thiodicarb, Thiofanox, Triazamate, Trimethacarb, XMC, Xylylcarb) and Organophosphates (such as Acephate, Azamethiphos, Azinphos-ethyl, Azinphosmethyl, Cadusafos, Chlorethoxyfos, Chlorfenvinphos, Chlormephos, Chlorpyrifos, Chlorpyrifos-methyl, Coumaphos, Cyanophos, Demeton-S-methyl, Diazinon, Dichlorvos/DDVP, Dicrotophos, Dimethoate, Dimethylvinphos, Disulfoton, EPN, Ethion, Ethoprophos, Famphur, Fenamiphos, Fenitrothion, Fenthion, Fosthiazate, Heptenophos, Imicyafos, Isofenphos, Isopropyl O-(methoxyaminothio-phosphoryl) salicylate, Isoxathion, Malathion, Mecarbam, Methamidophos, Methidathion, Mevinphos, Monocrotophos, Naled, Omethoate, Oxydemeton-methyl, Parathion, Parathion-methyl, Phenthoate, Phorate, Phosalone, Phosmet, Phosphamidon, Phoxim, Pirimiphos-methyl, Profenofos, Propetamphos, Prothiofos, Pyraclofos, Pyridaphenthion, Quinalphos, Sulfotep, Tebupirimfos, Temephos, Terbufos, Tetrachlorvinphos, Thiometon, Triazophos, Trichlorfon, Vamidothion). Examples of GABA-gated chloride channel blockers include without limitation Cyclodiene Organochlorines (such as Chlordane, Endosulfan) and Phenylpyrazoles (Fiproles) (such as Ethiprole, Fipronil). Examples of sodium channel modulators include without limitation pyrethroids and pyrethrins (such as Acrinathrin, Allethrin, d-cis-trans Allethrin, d-trans Allethrin, Bifenthrin, Bioallethrin, Bioallethrin Scyclopentenyl isomer, Bioresmethrin, Cycloprothrin, Cyfluthrin, beta-Cyfluthrin, Cyhalothrin, lambda-Cyhalothrin, gamma-Cyhalothrin, Cypermethrin, alpha-Cypermethrin, beta-Cypermethrin, thetacypermethrin, zeta-Cypermethrin, Cyphenothrin, (1R)-trans-isomers], Deltamethrin, Empenthrin (EZ)-(1R)-isomers], Esfenvalerate, Etofenprox, Fenpropathrin, Fenvalerate, Flucythrinate, Flumethrin, tau-Fluvalinate, Halfenprox, Imiprothrin, Kadethrin, Permethrin, Phenothrin [(1R)-trans-isomer], Prallethrin, Pyrethrins (pyrethrum), Resmethrin, Silafluofen, Tefluthrin, Tetramethrin, Tetramethrin [(1R)-isomers], Tralomethrin, Transfluthrin) and Methoxychlor. Examples of nAChR competitive modulators include without limitation Neonicotinoids (such as Acetamiprid, Clothianidin, Dinotefuran, Imidacloprid, Nitenpyram, Thiacloprid, Thiamethoxam), nicotine, sulfoximines (such as Sulfoxaflor), Butenolides (such as Flupyradifurone), and Mesoionics (such as Triflumezopyrim). Examples of nAChR allosteric modulators-Site I include without limitation Spinosyns (such as Spinetoram, Spinosad). Examples of GluCI allosteric modulators include without limitation Avermectins and Milbemycins (such as Abamectin, Emamectin benzoate, Lepimectin, Milbemectin). Examples of mult-site inhibitors include without limitation Alkyl halides (such as Methyl bromide and other alkyl halides), Chloropicrin, Fluorides (such as Cryolite (Sodium aluminum fluoride), Sulfuryl fluoride), Borates (such as Borax, Boric acid, Disodium octaborate, Sodium borate, Sodium metaborate), Tartar emetic, Methyl isothiocyanate generators (such as Dazomet, Metam). Examples of chordotonal organ TRPV channel modulators include without limitation Pyridine azomethine derivatives (such as Pymetrozine, Pyrifluquinazon), and Pyropenes (such as Afidopyropen). Examples of juvenile hormone mimics include without limitation juvenile hormone analogues (such as Hydroprene, Kinoprene, Methoprene), fenoxycarb, and pyriproxyfen. Examples of mite growth inhibitors affecting CHS1 include without limitation Clofentezine, Diflovidazin, Hexythiazox, and Etoxazole. Examples of microbial disruptors of insect midgut membranes include without limitation Bacillus thuringiensis (such as Bacillus thuringiensis subsp. israelensis, Bacillus thuringiensis subsp. aizawai, Bacillus thuringiensis subsp. kurstaki, Bacillus thuringiensis subsp. tenebrionis, Bacillus thuringiensis strain EX297512) and the insecticidal proteins they produce (such as Cry1Ab, Cry1Ac, Cry1Fa, Cry1A.105, Cry2Ab, Vip3A, mCry3A, Cry3Ab, Cry3Bb, Cry34Ab1/Cry35Ab1) and Bacillus sphaericus. Examples of inhibitors of mitochondrial ATP synthase include without limitation Diafenthiuron, Organotin miticides (such as Azocyclotin, Cyhexatin, Fenbutatin oxide), Propargite, and Tetradifon. Examples of uncouplers of oxidative phosphorylation via disruption of the proton gradient include without limitation Pyrroles (such as Chlorfenapyr), Dinitrophenols, and Sulfluramid. Examples of nAChR channel blockers include without limitation Nereistoxin analogues (such as Bensultap, Cartap hydrochloride, Thiocyclam, Thiosultap-sodium). Examples of inhibitors of chitin biosynthesis affecting CHS1 include without limitation Benzoylureas (such as Bistrifluron, Chlorfluazuron, Diflubenzuron, Flucycloxuron, Flufenoxuron, Hexaflumuron, Lufenuron, Novaluron, Noviflumuron, Teflubenzuron, Triflumuron). Examples of inhibitors of chitin biosynthesis-type 1 include without limitation Buprofezin. Examples of molting disruptors (Dipteran) include without limitation Cyromazine. Examples of ecdysone receptor agonists include without limitation Diacylhydrazines (such as Chromafenozide, Halofenozide, Methoxyfenozide, Tebufenozide). Examples of octopamine receptor agonists include without limitation Amitraz. Examples of mitochondrial complex III electron transport inhibitors include without limitation Hydramethylnon, Acequinocyl, Fluacrypyrim, and Bifenazate. Examples of mitochondrial complex I electron transport inhibitors include without limitation METI acaricides and insecticides such as Fenazaquin, Fenpyroximate, Pyridaben, Pyrimidifen, Tebufenpyrad, Tolfenpyrad) and Rotenone. Examples of voltage-dependent sodium channel blockers include without limitation Oxadiazines (such as Indoxacarb) and Semicarbazones (such as Metaflumizone). Examples of inhibitors of acetyl CoA carboxylase include without limitation Tetronic and Tetramic acid derivatives (such as Spirodiclofen, Spiromesifen, Spiropidion, Spirotetramat). Examples of mitochondrial complex IV electron transport inhibitors include without limitation Phosphides (Aluminium phosphide, Calcium phosphide, Phosphine, Zinc phosphide), Cyanides (such as Calcium cyanide, Potassium cyanide, Sodium cyanide). Examples of mitochondrial complex II electron transport inhibitors include without limitation Beta-ketonitrile derivatives (such as Cyenopyrafen, Cyflumetofen) and Carboxanilides (such as Pyflubumide). Examples of ryanodine receptor modulators include without limitation such as Diamides (such as Chlorantraniliprole, Cyantraniliprole, Cyclaniliprole Flubendiamide, Tetraniliprole). Examples of chordotonal organ modulators include without limitation Flonicamid. Examples of GABA-gated chloride channel allosteric modulators include without limitation Meta-diamides (Broflanilide) and Isoxazolines (such as Fluxametamide). Examples of nicotinic acetylcholine receptor (nAChR) Allosteric Modulators-Site II include without limitation GS-omega/kappa HXTX-Hv1a peptide.

In some embodiments, the synthetic composition comprises one or more endophytes of the present invention and one or more chemical or biological agent capable of killing, impeding the feeding and or growth and or reproduction of, repelling, and or reducing the severity or extent of infection to a plant host of, a pathogen of a plant, including without limitation chemical or biological agents that are PhenylAmides fungicides (acylalanines, oxazolidinones, butyrolactones), hydroxy-(2-amino-) pyrimidines, heteroaromatics (such as isoxazoles, isothiazolones), carboxylic acids, Methyl-Benzimidazole-Carbamates (MBC) fungicides (such as thiophanates, benzimidazoles), N-phenyl carbamates, benzamides (such as toluamides, pyridinylmethyl-benzamides), thiazole carboxamide (such as ethylamino-thiazole-carboxamide), phenylureas, cyanoacrylates (such as aminocyanoacrylates), aryl-phenyl-ketones (such as benzophenone, benzoylpyridine), pyrimidinamines, pyrazole-MET1 (such as pyrazole-5-carboxamides), quinazoline, succinate-dehydrogenase inhibitors (SDHI) (such as phenyl-benzamides, phenyl-oxo-ethyl thiophene amide, pyridinyl-ethyl-benzamides, phenyl-cyclobutyl-pyridineamide, furan-carboxamides, oxathiin-carboxamides, thiazole-carboxamides, pyrazole-4-carboxamides, N-cyclopropyl-N-benzyl-pyrazole-carboxamides, N-methoxy-(phenyl-ethyl)-pyrazole-carboxamides, pyridine-carboxamides, pyrazine-carboxamides, pydiflumetofen, fluxapyroxad), quinone outside inhibitors (such as methoxy-acrylates, methoxy-acetamide, methoxy-carbamates, oximino-acetates, oximino-acetamides, oxazolidine-diones, dihydro-dioxazines, imidazolinones, benzyl-carbamates, tetrazolinones), quinone inside inhibitors (such as cyano-imidazole, sulfamoyl-triazole, picolinamides), uncouplers of oxidative phosphorylation (such as dinitrophenyl-crotonates, 2,6-dinitro-anilines), organo tin compounds (tri-phenyl tin compounds), thiophene-carboxamides, Quinone outside Inhibitor-stigmatellin binding type (such as triazolo-pyrimidylamine), anilino-pyrimidines, enopyranuronic acid antibiotic, hexopyranosyl antibiotic, glucopyranosyl antibiotic, tetracycline antibiotic, aza-naphthalenes (such as aryloxyquinoline, quinazolinone), phenylpyrroles, dicarboximides, phosphoro-thiolates, dithiolanes, aromatic hydrocarbons, chlorophenyls, nitroanilines, heteroaromatics (such as 1,2,4-thiadiazoles), carbamates, demethylation inhibitors (such as piperazines, pyridines, pyrimidines, imidazoles, triazoles, triazolinthiones), amines (such as morpholines, piperidines, spiroketal-amines), ketoreductase inhibitors (such as hydroxyanilides, amino-pyrazolinone), thiocarbamates, allylamines, polyoxins (such as peptidyl pyrimidine nucleoside), Carboxylic Acid Amides (such as cinnamic acid amides, valinamide carbamates, mandelic acid amides), melanin biosynthesis inhibitors-reductase (such as isobenzo-furanone, pyrrolo-quinolinone, triazolobenzo-thiazole), melanin biosynthesis inhibitors-dehydratase (such as cyclopropane-carboxamide, carboxamide, propionamide), melanin biosynthesis inhibitors-polyketide synthase (such as trifluoroethyl-carbamate), benzo-thiadiazole, benzisothiazole, thiadiazole-carboxamide, polysaccharides (such as laminarin), plant ethanol extracts (such as anthraquinones, resveratrol, extract from Reynoutria sachalinensis), phosphonates (such as ethyl phosphonates, fosetyl-Al, phosphorous acid and salts), isothiazole (such as isothiazolylmethyl ether), cyanoacetamide-oxime, phthalamic acids, benzotriazines, benzene-sulphonamides, pyridazinones, phenyl-acetamide, guanidines, thiazolidine (such as cyano-methylene-thiazolidines), pyrimidinone-hydrazones, 4-quinolyl-acetates, tetrazolyloximes, glucopyranosyl antibiotics, copper salts, sulphur, dithio-carbamates and relatives (such as amobam, ferbam, mancozeb, maneb, metiram, propineb, thiram, zinc thiazole, zineb, ziram), phthalimides, chloronitriles (phthalonitriles), sulfamides (such as dichlofluanid, tolylfluanid), bis-guanidines (such as guazatine, iminoctadine), triazines (such as anilazine), quinones (anthraquinones) (such as dithianon), quinoxalines (such as chinomethionat, quinomethionate), maleimide (such as fluoroimide), thiocarbamate (such as methasulfocarb), polypeptide (lectin) plant extracts (such as extract from the cotyledons of lupine plantlets), phenol and sesquiterpene and triterpenoid and coumarin plant extracts (such as extract from Swinglea glutinosa), terpene hydrocarbon and terpene alcohol and terpene phenol extracts plant extracts (such as extract from Melaleuca alternifolia, plant oils such as eugenol, geraniol, thymol mixtures thereof), Polyene (such as amphoteric macrolide antifungal antibiotic from Streptomyces natalensis or Streptomyces chattanoogensis), oxysterol binding protein homologue inhibition (piperidinyl-thiazole-isoxazolines), other active compounds (such as Fludioxonil, Mefenoxam, Sedaxane, Azoxystrobin, Thiabendazole, Ethaboxam, metalaxyl, Trifloxystrobin, Myclobutanil, Acibenzolar-S-methyl, Metconazole, tolclofos-methyl, Fluopyram, Ipconazole, Oxathiapiprolin, Difenoconazole, Prothyoconazol, Tebuconazole, Pyraclostrobin, Fluxapyroxad), and combinations thereof.

In some embodiments, the synthetic composition comprises one or more endophytes of the present invention and one or more biological agents (for example bacterial or fungal agents) including, but not limited to, those agents capable of killing, impeding the feeding and or growth and or reproduction of, repelling, and or reducing the severity or extent of infection to a plant host of, a pathogen or pest of a plant. The one or more bacterial or fungal agents may be living or dead (including without limitation by heat inactivation) bacteria or fungi, extracts and or metabolites of bacteria or fungi (including without limitation extracts and or metabolites in spent growth media), or combinations thereof. Non-limiting examples of biological agents include Trichoderma species including without limitation Trichoderma atroviride strain I-1237, Trichoderma atroviride strain LU132, Trichoderma atroviride strain SC1, Trichoderma atroviride strain SKT-1, Trichoderma atroviride strain 77B, Trichoderma asperellum strain T34, Trichoderma asperellum strain kd, Trichoderma harzianum strain T-22, Trichoderma virens strain G-41; Clonostachys species including without limitation Gliocladium catenulatum strain J1446, Clonostachys rosea strain CR-7; Coniothyrium species including without limitation Coniothyrium minitans strain CON/M/91-08; Talaromyces species including without limitation Talaromyces flavus strain SAY-Y-94-01; Saccharomyces species including without limitation Saccharomyces cerevisae strain LAS02; Bacillus species including without limitation Bacillus amyloliquefaciens strain QST713, Bacillus amyloliquefaciens strain FZB24, Bacillus amyloliquefaciens strain MBI600, Bacillus amyloliquefaciens strain D747, Bacillus amyloliquefaciens strain F727, Bacillus amyloliquefaciens strain AT-332, Bacillus amyloliquefaciens strain MBI 600 Bacillus mycoides isolate J, Bacillus subtilis strain AFS032321, Bacillus subtilis strain Y1336, Bacillus subtilis strain HAI-0404); Pseudomonas species including without limitation Pseudomonas chlororaphis strain AFS009; Streptomyces species including without limitation Streptomyces griseovirides strain K61, Streptomyces lydicus strain WYEC108; Penicillium species such as Penicillium bilaiae, Penicillium bilaiae; Pasteuria species including without limitation Pasteuria nishizawae Pnl),

In some embodiments, one or more endophytes of the present invention and one more or chemical or biological agents described herein are present in a synthetic composition at a weight ratio of between 1000:1 and 1:1000, 100:1 and 1:100, or 10:1 and 1:10.

In some embodiments, the synthetic composition may be stored at between 0 degrees Celsius and 4 degrees Celsius for 1 week with less than 1 log loss of CFU of the one or more endophytes. In some embodiments, the synthetic composition may be stored at between 4.1 degrees Celsius and 20 degrees Celsius for 1 week with less than 1 log loss of CFU of the one or more endophytes. In some embodiments, the synthetic composition may be stored at between 20.1 degrees Celsius and 33 degrees Celsius for 1 week with less than 1 log loss of CFU of the one or more endophytes.

In yet another aspect, described herein are methods of measuring plant health comprising determining the presence or abundance of one or more endophytes in a plant element, growth medium or growth environment, wherein the one or more endophytes comprise at least one polynucleotide sequence that is at least 97% identical to SEQ ID NO. 5. In some embodiments, the presence or abundance of one or more endophytes is determined relative to a reference plant element, growth medium or growth environment. In some embodiments, the one or more endophytes are not present in the reference plant element, growth medium or growth environment. In some embodiments, the one or more endophytes are less abundant in the reference plant element, growth medium or growth environment. In some embodiments, the presence or abundance of one or more endophytes is determined in a plant element and modulation of one or more traits of agronomic importance is inferred from the presence or amount of the one or more endophytes in the plant element. In some embodiments, the presence or abundance of one or more endophytes is determined in a growth medium and the capacity of the growth medium to modulate one or more trait of agronomic importance in a plant element planted therein is inferred from the presence or amount of the one or more endophytes in the growth medium. In some embodiments, the presence or abundance of one or more endophytes is determined in a growth environment and the capacity of the growth environment to modulate one or more trait of agronomic importance in a plant element grown therein is inferred from the presence or amount of the one or more endophytes in the growth environment. In some embodiments, the presence or abundance of one or more endophytes is determined by polymerase chain reaction, fluorescence in situ hybridization, or isothermal amplification.

In some embodiments, a plurality of nucleic acid probes are used to determine the presence or abundance of one or more endophytes in a plant element, growth medium or growth environment, wherein the plurality comprises complementary or reverse complementary sequences to a region of at least 10 contiguous nucleotides within SEQ ID NO. 5. In some embodiments, the complementary or reverse complementary region comprises at least 20 contiguous nucleotides. In some embodiments, the complementary or reverse complementary region comprises at least 30 contiguous nucleotides. In some embodiments, the complementary or reverse complementary region comprises at least 40 contiguous nucleotides. In some embodiments, the plurality of nucleic acid probes are single-stranded DNA. In some embodiments, the plurality of nucleic acid probes are attached to one or more solid supports. In some embodiments, the plurality of nucleic acid probes are attached to a plurality of beads. In some embodiments, the plurality of nucleic acid probes are attached to a contiguous solid support.

In some embodiments, the plant element is a monocot. In some embodiments, the monocot is a cereal. In some embodiments, the cereal is selected from the group consisting of wheat, rice, barley, buckwheat, rye, millet, oats, corn, sorghum, triticale, and spelt. In some embodiments, the cereal is wheat.

In some embodiments, the plant element is a dicot. In some embodiments, the dicot is selected from the group consisting of cotton, tomato, lettuce, peppers, cucumber, endive, melon, potato, and squash. In some embodiments, the dicot is a legume. In some embodiments, the legume is soy, peas, or beans.

In some embodiments, the plant element is a whole plant, seedling, meristematic tissue, ground tissue, vascular tissue, dermal tissue, seed, leaf, root, shoot, stem, flower, fruit, stolon, bulb, tuber, corm, keikis, shoot, or bud. In some embodiments, the plant element is a seed.

In some embodiments, the trait of agronomic importance is selected from the group consisting of yield, root weight, shoot weight, plant height, full emergence, biotic stress tolerance, and combinations thereof. In some embodiments, the trait of agronomic importance is biotic stress tolerance. In some embodiments, the trait of agronomic importance is improved nutrient use efficiency. In some embodiments, the trait of agronomic importance is drought tolerance.

In some embodiments, the one or more endophytes is a member of the Class Bacilli. In some embodiments, the one or more endophytes is a member of the Order Bacillales. In some embodiments, the one or more endophytes is a member of the Family Bacillaceae. In some embodiments, the one or more endophytes is a member of the Genus Bacillus.

In some embodiments, the one or more endophytes comprises at least 2 endophytes. In some embodiments, the one or more endophytes comprises at least 3 endophytes. In some embodiments, the one or more endophytes comprises at least 4 endophytes. In some embodiments, the one or more endophytes comprises at least 5 endophytes. In some embodiments, the one or more endophytes comprises at least 10 endophytes.

In some embodiments, the one or more endophytes are encapsulated in polymeric beads. In some embodiments, the polymeric beads are less than 500 μm in diameter at their widest point. In some embodiments, the polymeric beads are less than 200 μm in diameter at their widest point. In some embodiments, the polymeric beads are less than 100 μm in diameter at their widest point. In some embodiments, the polymeric beads are less than 50 μm in diameter at their widest point. In some embodiments, the polymeric beads' average diameter at their widest point is between 500 μm and 250 μm. In some embodiments, the polymeric beads' average diameter at their widest point is between 249 μm and 100 μm. In some embodiments, the polymeric beads' average diameter at their widest point is between 100 μm and 50 μm.

### DETAILED DESCRIPTION

Terms used in the claims and specification are defined as set forth below unless otherwise specified.

It must be noted that, as used in the specification and the appended claims, the singular forms “a,” “an” and “the” include plural referents unless the context clearly dictates otherwise.

This invention relates to methods and compositions for improving plant health. The present invention includes methods for improving plant health, as well as synthetic compositions comprising endophytes capable of improving plant health, and nucleic acid probes and nucleic acid detection kits that may be used to identify endophytes of the present invention.

“Plant health” is demonstrated by the improvement of a trait of agronomic importance in a plant or plant element as compared to a reference plant or plant element. A trait of agronomic importance includes, but is not limited to, drought tolerance, heat tolerance, cold tolerance, salinity tolerance, metal tolerance, herbicide tolerance, improved water use efficiency, improved nitrogen utilization, improved nitrogen fixation, improved nutrient use efficiency, improved nutrient utilization, biotic stress tolerance, yield improvement, health enhancement, vigor improvement, decreased necrosis, decreased chlorosis, decreased area of necrotic tissue, decreased area of chlorotic tissue, decreased pathogen load of tissues, growth improvement, photosynthetic capability improvement, nutrition enhancement, altered protein content, altered oil content, increased biomass, increased shoot height, increased root length, increased shoot biomass, increased root biomass, increased leaf area, increased shoot area, increased root area, improved root architecture, increased seed germination percentage, increased seed germination rate, increased seedling survival, increased survival, photosynthetic efficiency, transpiration rate, seed/fruit number or mass, plant grain or fruit yield, leaf chlorophyll content, photosynthetic rate, wilt recovery, turgor pressure, modulation of a metabolite, production of a volatile organic compound (VOC), modulation of the proteome, increased seed weight, altered seed carbohydrate composition, altered seed oil composition, altered seed protein composition, altered seed nutrient composition, and combinations thereof. The phrase “biotic stress” refers to a growth environment comprising one or more pests or pathogens. Pests can be nematodes and/or insects. In some embodiments, a pest is of the order Lepidoptera, Hemiptera, Tylenchida/Rhabditida, Dorylaimida, Heterodera, Meloidogyne, Pratylenchus, Trichinellida, Globodera, Xiphinema, Hoplolaimus, Longidorus, Rotylenchulus, Helicotylenchus, Belonolaimus, Trichodorus, Paratrichodorus, Tylenchorhynchus, Anguillulina, Merlinia, or Triplonchida. Pathogens can be fungal, viral, protist, or bacterial pathogens, for example of vertebrates or plants. In some embodiments, a pathogen is of a genera Pythium, Rhizoctonia, Phytophthora, Fusarium, Alternaria, Stagonospora, Aspergillus, Magnaporthe, Botrytis, Puccinia, Blumeria, Erysiphe, Leveillula, Mycosphaerella, Colletotrichum, Macrophomina, Cercospora, Corynespora, or Phomopsis. “Biomass” means the total mass or weight (fresh or dry), at a given time (for example, age or stage of development), of a plant tissue, plant tissues, an entire plant, or population of plants. The term may also refer to all the plants or species in the community (“community biomass”).

An “increased yield” can refer to any increase in seed or fruit biomass; or seed, seed pod or ear, or fruit number per plant; or seed or fruit weight; or seed or fruit size per plant or unit of production area, e.g. acre or hectare. For example, increased yield of seed or fruit biomass may be measured in units of bushels per acre, pounds per acre, tons per acre, or kilos per hectare. An increased yield can also refer to an increased production of a component of, or product derived from, a plant or plant element or of a unit of measure thereof. For example, increased carbohydrate yield of a grain or increased oil yield of a seed. Typically, where yield indicates an increase in a particular component or product derived from a plant, the particular characteristic is designated when referring to increased yield, e.g., increased oil or grain yield or increased protein yield or seed size.

“Nutrition enhancement” refers to modulation of the presence, abundance or form of one or more substances in a plant element, wherein the modulation of the one or more substances provides a benefit to other organisms that consume or utilize said plant element.

Synthetic compositions and methods of use described herein may improve plant health by providing an improved benefit or tolerance to a plant that is of at least 0.1%, at least 0.5%, at least 1%, at least 2%, at least 3%, between 3% and 5%, at least 5%, between 5% and 10%, at least 10%, between 10% and 15%, at least 15%, between 15% and 20%, at least 20%, between 20% and 30%, at least 30%, between 30% and 40%, at least 40%, between 40% and 50%, at least 50%, between 50% and 60%, at least 60%, between 60% and 75%, at least 75%, between 75% and 100%, at least 100%, between 100% and 150%, at least 150%, between 150% and 200%, at least 200%, between 200% and 300%, at least 300% or more, when compared with a reference plant. A “reference plant”, “reference plant element”, “reference agricultural plant” or “reference seed” means a similarly situated plant or seed of the same species, strain, or cultivar to which a treatment, formulation, composition or endophyte preparation as described herein is not administered/contacted. A reference plant, therefore, is identical to the treated plant except for the presence of the active ingredient to be tested and can serve as a control for detecting the effects of the treatment conferred to the plant. A plurality of reference plants may be referred to as a “reference population”.

In some embodiments, one or more endophytes and or one or more compounds produced by one or more endophytes are heterologously disposed on a plant element in an effective amount to improve plant health. In some embodiments, an improvement of plant health is measured by an increase in a trait of agronomic importance, for example root length or yield. In some embodiments, an improvement of subject health is measured by a decrease in a trait of importance, for example necrosis or chlorosis. In some embodiments, improved plant health is demonstrated by an improvement of a trait of agronomic importance or tolerance in a treated plant by at least 0.1%, at least 0.5%, at least 1%, at least 2%, at least 3%, between 3% and 5%, at least 5%, between 5% and 10%, at least 10%, between 10% and 15%, at least 15%, between 15% and 20%, at least 20%, between 20% and 30%, at least 30%, between 30% and 40%, at least 40%, between 40% and 50%, at least 50%, between 50% and 60%, at least 60%, between 60% and 75%, at least 75%, between 75% and 100%, at least 100%, between 100% and 150%, at least 150%, between 150% and 200%, at least 200%, between 200% and 300%, at least 300% or more, as compared to a reference plant element not further comprising said endophyte. An “effective amount” of one or more endophytes is the amount capable of improving trait of agronomic importance or tolerance by at least 0.1%, at least 0.5%, at least 1%, at least 2%, at least 3%, between 3% and 5%, at least 5%, between 5% and 10%, at least 10%, between 10% and 15%, at least 15%, between 15% and 20%, at least 20%, between 20% and 30%, at least 30%, between 30% and 40%, at least 40%, between 40% and 50%, at least 50%, between 50% and 60%, at least 60%, between 60% and 75%, at least 75%, between 75% and 100%, at least 100%, between 100% and 150%, at least 150%, between 150% and 200%, at least 200%, between 200% and 300%, at least 300% or more, as compared to a reference plant element not further comprising said endophyte. In some embodiments, an effective amount of treatment comprising an endophyte is at least 10 CFU per unit of plant element, at least 10\hat{ }2 CFU per unit of plant element, between 10\hat{ }2 and 10\hat{ }3 CFU per unit of plant element, at least about 10\hat{ }3 CFU per unit of plant element, between 10\hat{ }3 and 10\hat{ }4 CFU per unit of plant element, at least about 10\hat{ }4 CFU per unit of plant element, between 10\hat{ }4 and 10\hat{ }5 CFU per unit of plant element, at least about 10\hat{ }5 CFU, between 10\hat{ }5 and 10\hat{ }6 CFU per unit of plant element, at least about 10\hat{ }6 CFU per unit of plant element, between 10\hat{ }6 and 10\hat{ }7 CFU per unit of plant element, at least about 10\hat{ }7 CFU per unit of plant element, between 10\hat{ }7 and 10\hat{ }8 CFU per unit of plant element, or even greater than 10\hat{ }8 CFU per unit of plant element. A unit of a plant element may be an individual plant element, e.g. an individual seed, or a unit of area surface area of a plant element, e.g. a square inch of leaf tissue, or unit of surface area of a plant element, e.g. a cubic centimeter of root.

In some embodiments, an effective amount of treatment comprising an endophyte is at least 10 CFU per gram of synthetic composition, at least 10\hat{ }2 CFU per gram of synthetic composition, between 10\hat{ }2 and 10\hat{ }3 CFU per gram of synthetic composition, at least about 10\hat{ }3 CFU per gram of synthetic composition, between 10\hat{ }3 and 10\hat{ }4 CFU per gram of synthetic composition, at least about 10\hat{ }4 CFU per gram of synthetic composition, between 10\hat{ }4 and 10\hat{ }5 CFU per gram of synthetic composition, at least about 10\hat{ }5 CFU per gram of synthetic composition, between 10\hat{ }5 and 10\hat{ }6 CFU per gram of synthetic composition, at least about 10\hat{ }6 CFU per gram of synthetic composition, between 10\hat{ }6 and 10\hat{ }7 CFU per gram of synthetic composition, at least about 10\hat{ }7 CFU per gram of synthetic composition, between 10\hat{ }7 and 10\hat{ }8 CFU per gram of synthetic composition, at least about 10\hat{ }8 CFU per gram of synthetic composition, between 10\hat{ }8 and 10\hat{ }9 CFU per gram of synthetic composition, at least about 10\hat{ }9 CFU per gram of synthetic composition, between 10\hat{ }9 and 10\hat{ }10 CFU per gram of synthetic composition, or even greater than 10\hat{ }10 CFU per unit of plant element. In some embodiments, a synthetic composition comprises an endophyte heterologously disposed in a treatment formulation at concentration of at least 10\hat{ }6 CFU per gram.

In some embodiments, an effective amount of treatment comprising an endophyte is at least 10 CFU per unit of plant element, at least 10\hat{ }2 CFU per unit of plant element, between 10\hat{ }2 and 10\hat{ }3 CFU per unit of plant element, at least about 10\hat{ }3 CFU per unit of plant element, between 10\hat{ }3 and 10\hat{ }4 CFU per unit of plant element, at least about 10\hat{ }4 CFU per unit of plant element, between 10\hat{ }4 and 10\hat{ }5 CFU per unit of plant element, at least about 10\hat{ }5 CFU per unit of plant element, between 10\hat{ }5 and 10\hat{ }6 CFU per unit of plant element, at least about 10\hat{ }6 CFU per unit of plant element, between 10\hat{ }6 and 10\hat{ }7 CFU per unit of plant element, at least about 10\hat{ }7 CFU per unit of plant element, between 10\hat{ }7 and 10\hat{ }8 CFU per unit of plant element, at least about 10\hat{ }8 CFU per unit of plant element, between 10\hat{ }8 and 10\hat{ }9 CFU per unit of plant element, at least about 10\hat{ }9 CFU per unit of plant element, between 10\hat{ }9 and 10\hat{ }10 CFU per unit of plant element, or even greater than 10\hat{ }10 CFU per unit of plant element. In some embodiments, the plant element is a seed.

The methods and compositions of the present invention are broadly applicable to cultivated plants, particularly plants that are cultivated by humans for food, feed, fiber, fuel, and/or industrial purposes. In some embodiments, plants (including seeds and other plant elements) are monocots or dicots. In some embodiments, plants used in the methods and compositions of the present invention include, but are not limited to: agricultural row, agricultural grass plants or other field crops: wheat, rice, barley, buckwheat, beans (for example: soybean, snap, dry), corn (for example: grain, seed, sweet corn, silage, popcorn, high oil), canola, peas (for example: dry, succulent), peanuts, safflower, sunflower, alfalfa hay, forage and cover crops (for example: alfalfa, clover, vetch, and trefoil), berries and small fruits (for example: blackberries, blueberries, currants, elderberries, gooseberries, huckleberries, loganberries, raspberries, strawberries, bananas and grapes), bulb crops (for example: garlic, leeks, onions, shallots, and ornamental bulbs), citrus fruits (for example: citrus hybrids, grapefruit, kumquat, lines, oranges, and pummelos), cucurbit vegetables (for example: cucumbers, melons, gourds, pumpkins, and squash), flowers (for example: ornamental, horticultural flowers including roses, daisies, tulips, freesias, carnations, heather, lilies, irises, orchids, snapdragons, and ornamental sunflowers), bedding plants, ornamentals, fruiting vegetables (for example: eggplant, sweet and hot peppers, tomatillos, and tomatoes), herbs, spices, mints, hydroponic crops (for example: cucumbers, tomatoes, lettuce, herbs, and spices), leafy vegetables and cole crops (for example: arugula, celery, chervil, endive, fennel, lettuce including head and leaf, parsley, radicchio, rhubarb, spinach, Swiss chard, broccoli, Brussels sprouts, cabbage, cauliflower, collards, kale, kohlrabi, and mustard greens), asparagus, legume vegetable and field crops (for example: snap and dry beans, lentils, succulent and dry peas, and peanuts), pome fruit (for example: pears and quince), root crops (for example: beets, sugar beets, red beets, carrots, celeriac, chicory, horseradish, parsnip, radish, rutabaga, salsify, and turnips), deciduous trees (for example: maple and oak), evergreen trees (for example: pine, cedar, hemlock and spruce), small grains (for example: rye, wheat including spring and winter wheat, millet, oats, barley including spring and winter barley, and spelt), stone fruits (for example: apricots, cherries, nectarines, peaches, plums, and prunes), tree nuts (for example: almonds, beech nuts, Brazil nuts, butternuts, cashews, chestnuts, filberts, hickory nuts, macadamia nuts, pecans, pistachios, and walnuts), oil seed crops (for example: soybeans, sunflowers, canola, copra, cottonseed, palm kernel, peanut, rapeseed, and flax), and tuber crops (for example: potatoes, sweet potatoes, yams, artichoke, cassava, and ginger). In a particular embodiment, the agricultural plant is selected from the group consisting of rice (Oryza sativa and related varieties), soy (Glycine max and related varieties), wheat (Triticum aestivum and related varieties), oats (Avena sativa and related varieties), barley (Hordeum vulgare and related varieties), corn (Zea mays and related varieties), peanuts (Arachis hypogaea and related varieties), canola (Brassica napus, Brassica rapa and related varieties), coffee (Coffea spp.), cocoa (Theobroma cacao), melons, and tomatoes (Solanum lycopsersicum and related varieties).

Plant health may be improved by treatment of a plant or plant element. A “plant element” is intended to generically reference either a whole plant or a plant component, including but not limited to plant tissues, parts, and cell types. A plant element is preferably one of the following: whole plant, seedling, meristematic tissue, ground tissue, vascular tissue, dermal tissue, seed, leaf, root, shoot, stem, flower, fruit, stolon, bulb, tuber, corm, keikis, shoot, or bud.

Plant health may be improved by treatment with a composition of the present invention, in particular compositions of the present invention comprising one or more endophytes. An “endophyte” is an organism capable of living on a plant element (e.g., rhizoplane or phyllosphere) or within a plant element, or on a surface in close physical proximity with a plant element, e.g., the phyllosphere and rhizosphere including soil surrounding roots. A “beneficial” endophyte does not cause disease or harm the host plant otherwise. Endophytes can occupy the intracellular or extracellular spaces of plant tissue, including the leaves, stems, flowers, fruits, seeds, or roots. An endophyte can be, for example, a bacterial or fungal organism, and can confer a beneficial property to the host plant such as an increase in yield, biomass, resistance, or fitness. An endophyte can be a fungus or a bacterium. As used herein, the term “microbe” is sometimes used to describe an endophyte. As used herein, the term “microbe” or “microorganism” refers to any species or taxon of microorganism, including, but not limited to, archaea, bacteria, microalgae, fungi (including mold and yeast species), mycoplasmas, microspores, nanobacteria, oomycetes, and protozoa. In some embodiments, a microbe or microorganism is an endophyte, for example a bacterial or fungal endophyte, which is capable of living within a plant.

The term “isolated” is intended to specifically reference an organism, cell, tissue, polynucleotide, or polypeptide that is removed from its original source and purified from additional components with which it was originally associated. For example, an endophyte may be considered isolated from a seed if it is removed from that seed source and purified so that it is isolated from one or more additional components with which it was originally associated. Similarly, an endophyte may be removed and purified from a plant or plant element so that it is isolated and no longer associated with its source plant or plant element.

As used herein, an isolated strain of a microbe is a strain that has been removed from its natural milieu. “Pure cultures” or “isolated cultures” are cultures in which the organisms present are only of one strain of a particular genus and species. “Mixed cultures,” are cultures in which more than one genus and/or species of microorganism are present. As such, the term “isolated” does not necessarily reflect the extent to which the microbe has been purified. A “substantially pure culture” of the strain of microbe refers to a culture which contains substantially no other microbes than the desired strain or strains of microbe. In other words, a substantially pure culture of a strain of microbe is substantially free of other contaminants, which can include microbial contaminants. Further, as used herein, a “biologically pure” strain is intended to mean the strain was separated from materials with which it is normally associated in nature. A strain associated with other strains, or with compounds or materials that it is not normally found with in nature, is still defined as “biologically pure.” A monoculture of a particular strain is, of course, “biologically pure.” As used herein, the term “enriched culture” of an isolated microbial strain refers to a microbial culture that contains more that 50%, 60%, 70%, 80%, 90%, or 95% of the isolated strain.

A “population” of endophytes, or an “endophyte population”, refers to one or more endophytes that share a common genetic derivation, e.g., one or more propagules of a single endophyte, i.e., endophytes grown from a single picked colony. In some embodiments, a population refers to endophytes of identical taxonomy. In some embodiments, a population of endophytes refers to one or more endophytes of the same genus. In some embodiments, a population of endophytes refers to one or more endophytes of the same species or strain.

A “plurality of endophytes” means two or more types of endophyte entities, e.g., of bacteria or fungi, or combinations thereof. In some embodiments, the two or more types of endophyte entities are two or more individual endophytic organisms, regardless of genetic derivation or taxonomic relationship. In some embodiments, the two or more types of endophyte entities are two or more populations of endophytes. In other embodiments, the two or more types of endophyte entities are two or more species of endophytes. In yet other embodiments, the two or more types of endophyte entities are two or more genera of endophytes. In yet other embodiments, the two or more types of endophyte entities are two or more families of endophytes. In yet other embodiments, the two or more types of endophyte entities are two or more orders of endophytes. In yet other embodiments, the two or more types of endophyte entities are two or more classes of endophytes. In yet other embodiments, the two or more types of endophyte entities are two or more phyla of endophytes. In some embodiments, a plurality refers to three or more endophytes, either distinct individual organisms or distinct members of different genetic derivation or taxa. In some embodiments, a plurality refers to four or more either distinct individual endophytic organisms or distinct members of different genetic derivation or taxa. In some embodiments, a plurality refers to five or more, ten or more, or an even greater number of either distinct individual endophytic organisms or distinct members of different genetic derivation or taxa. In some embodiments, the term “consortium” or “consortia” may be used as a collective noun synonymous with “plurality”, when describing more than one population, species, genus, family, order, class, or phylum of endophytes.

In some embodiments, a treatment may comprise a modified microbe or plant or plant element. A microbe, plant, or plant element is “modified” when it comprises an artificially introduced genetic or epigenetic modification. In some embodiments, the modification is introduced by a genome engineering or genome editing technology. In some embodiments, genome engineering or editing utilizes non-homologous end joining (NHEJ), homology directed repair (HDR), or combinations thereof. In some embodiments, genome engineering or genome editing is carried out with a Class I or Class II clustered regulatory interspaced short palindromic repeats (CRISPR) system. In some embodiments, the CRISPR system is CRISPR/Cas9. In some embodiments, the CRISPR system is CRISPR/Cpf1. In some embodiments, the modification is introduced by a targeted nuclease. In some embodiments, targeted nucleases include, but are not limited to, transcription activator-like effector nuclease (TALEN), zinc finger nuclease (ZNF), Cas9, Cas9 variants, Cas9 homologs, Cpf1, Cpf1 variants, Cpf1 homologs, and combinations thereof. In some embodiments, the modification is an epigenetic modification. In some embodiments, the modification is introduced by treatment with a DNA methyltransferase inhibitor such as 5-azacytidine, or a histone deacetylase inhibitor such as 2-amino-7-methoxy-3H-phenoxazin-3-one. In some embodiments, the modification is introduced via tissue culture. In some embodiments, a modified microbe or plant or plant element comprises a transgene.

As used herein, the term “bacterium” or “bacteria” refers in general to any prokaryotic organism and may reference an organism from either Kingdom Eubacteria (Bacteria), Kingdom Archaebacteria (Archaea), or both. In some cases, bacterial genera have been reassigned due to various reasons (such as, but not limited to, the evolving field of whole genome sequencing), and it is understood that such nomenclature reassignments are within the scope of any claimed genus.

As used herein, the term “fungus” or “fungi” refers in general to any organism from Kingdom Fungi. Historical taxonomic classification of fungi has been according to morphological presentation. Beginning in the mid-1800's, it was recognized that some fungi have a pleomorphic life cycle, and that different nomenclature designations were being used for different forms of the same fungus. With the development of genomic sequencing, it became evident that taxonomic classification based on molecular phylogenetics did not align with morphological-based nomenclature (Shenoy B D, Jeewon R, Hyde K D. Impact of DNA sequence-data on the taxonomy of anamorphic fungi. Fungal Diversity 26 (10) 1-54. 2007). Systematics experts have not aligned on common nomenclature for all fungi, nor are all existing databases and information resources inclusive of updated taxonomies. As such, many fungi provided herein may be described by their anamorph form, but it is understood that based on identical genomic sequencing, any pleomorphic state of that fungus may be considered to be the same organism. In some cases, fungal genera have been reassigned due to various reasons, and it is understood that such nomenclature reassignments are within the scope of any claimed genus.

The degree of relatedness between microbes may be inferred from the sequence similarity of one or more homologous polynucleotide sequences of the microbes. In some embodiments, the one or more homologous polynucleotide sequences are marker genes. As used herein, the term “marker gene” refers to a conserved genomic region comprising sequence variation among related organisms. Examples of marker genes that may be used for the present invention, include but are not limited to: 16S ribosomal RNA gene (“16S”), internal transcribed spacer (“ITS”); fusA gene; largest subunit of RNA polymerase II (“RPB1”); second largest subunit of RNA polymerase II (“RPB2”); beta-tubulin or tubulin (“BTUB2” or “TUB2”); phosphoglycerate kinase (“PGK”); actin (“ACT”); long subunit rRNA gene (“LSU”); small subunit rRNA gene (“SSU”), 60S ribosomal protein L 10 (“60S_L10_L1”), atpD, Calmodulin (“CMD”), GDP gene (“GPD1_2”), etc.

The terms “sequence similarity”, “identity”, “percent identity”, “percent sequence identity” or “identical” in the context of polynucleotide sequences refer to the nucleotides in the two sequences that are the same when aligned for maximum correspondence. There are different algorithms known in the art that can be used to measure nucleotide sequence identity. Nucleotide sequence identity can be measured by a local or global alignment, preferably implementing an optimal local or optimal global alignment algorithm. For example, a global alignment may be generated using an implementation of the Needleman-Wunsch algorithm (Needleman, S. B. & Wunsch, C. D. (1970) Journal of Molecular Biology. 48 (3): 443-53). For example, a local alignment may be generated using an implementation of the Smith-Waterman algorithm (Smith T. F & Waterman, M. S. (1981) Journal of Molecular Biology. 147 (1): 195-197). Optimal global alignments using the Needleman-Wunsch algorithm and optimal local alignments using the Smith-Waterman algorithm are implemented in USEARCH, for example USEARCH version v8.1.1756_i86osx32.

A gap is a region of an alignment wherein a sequence does not align to a position in the other sequence of the alignment. A terminal gap is a region beginning at the end of a sequence in an alignment wherein the nucleotide in the terminal position of that sequence does not correspond to a nucleotide position in the other sequence of the alignment and extending for all contiguous positions in that sequence wherein the nucleotides of that sequence do not correspond to a nucleotide position in the other sequence of the alignment. An internal gap is a gap in an alignment which is flanked on the 3′ and 5′ end by positions wherein the aligned sequences are identical. In global alignments, terminal gaps are discarded before identity is calculated. For both local and global alignments, internal gaps are counted as differences.

In some embodiments, the nucleic acid sequence to be aligned is a complete gene. In some embodiments, the nucleic acid sequence to be aligned is a gene fragment. In some embodiments, the nucleic acid sequence to be aligned is an intergenic sequence. In a preferred embodiment, inference of homology from a sequence alignment is made where the region of alignment is at least 85% of the length of the query sequence.

The term “substantial homology” or “substantial similarity,” when referring to a polynucleotide sequence or fragment thereof, indicates that, when optimally aligned with appropriate nucleotide insertions or deletions with another polynucleotide sequence (or its complementary strand), there is nucleotide sequence identity in at least about 76%, 80%, 85%, or at least about 90%, or at least about 95%, 96%, at least 97%, 98%, 99% or 100% of the positions of the alignment, wherein the region of alignment is at least about 50%, 60%, 70%, 75%, 85%, or at least about 90%, or at least about 95%, 96%, 97%, 98%, 99% or 100% of the length of the query sequence. In a preferred embodiment, the region of alignment contains at least 100 positions inclusive of any internal gaps. In some embodiments, the region of alignment comprises at least 100 nucleotides of the query sequence. In some embodiments, the region of alignment comprises at least 200 nucleotides of the query sequence. In some embodiments, the region of alignment comprises at least 300 nucleotides of the query sequence. In some embodiments, the region of alignment comprises at least 400 nucleotides of the query sequence. In some embodiments, the region of alignment comprises at least 500 nucleotides of the query sequence. In some embodiments, the terminal nucleotides are trimmed from one or both ends of the sequence prior to alignment. In some embodiments, at least the terminal 10, 15, 20, 25, 30, between 20-30, 35, 40, 45, 50, between 25-50 nucleotides are trimmed from the sequence prior to alignment.

### Synthetic Compositions for Improving Plant Health

In some embodiments, a synthetic composition comprises one or more endophytes capable of improving plant health. A “synthetic composition” comprises one or more endophytes combined by human endeavor with a heterologously disposed plant element or a treatment formulation, said combination which is not found in nature. In some embodiments, a synthetic composition comprises one or more plant elements or formulation components combined by human endeavor with an isolated, purified endophyte composition. In some embodiments, synthetic composition refers to a plurality of endophytes in a treatment formulation comprising additional components with which said endophytes are not found in nature. An endophyte is “heterologously disposed” when mechanically or manually applied, artificially inoculated or disposed onto or into a plant element, seedling, plant or onto or into a plant growth medium or onto or into a treatment formulation so that the endophyte exists on or in the plant element, seedling, plant, plant growth medium, or formulation in a manner not found in nature prior to the application of the treatment, e.g., said combination which is not found in nature in that plant variety, at that time in development, in that tissue, in that abundance, or in that growth condition (for example, drought, flood, cold, nutrient deficiency, etc.).

A “treatment formulation” refers to one or more compositions that facilitate the stability, storage, and/or application of one or more endophytes. Treatment formulations may comprise any one or more agents such as: surfactant, a buffer, a tackifier, a microbial stabilizer, an antimicrobial, a fungicide, an anticomplex agent, an herbicide, a nematicide, an insecticide, a plant growth regulator, a rodenticide, a desiccant, a nutrient, an excipient, a wetting agent, a salt, a polymer. As used herein as a noun, a “treatment” may comprise one or more endophytes.

In some embodiments, a treatment formulation may comprise one or more polymeric beads comprising one or more endophytes. In some embodiments, a treatment formulation may consist of one or more polymeric beads comprising one or more endophytes. A polymeric bead may contain a biodegradable polymer such as alginate, agarose, agar, gelatin, polyacrylamide, chitosan, and polyvinyl alcohol. In some embodiments, the polymeric beads are less than 500 μm in diameter at their widest point. In some embodiments, the polymeric beads' average diameter at their widest point is between 500 μm and 250 μm, between 249 μm and 100 μm, 100 μm or less, between 100 μm and 50 μm, or 50 μm or less.

In some embodiments, an “agriculturally compatible carrier” can be used to formulate an agricultural formulation or other composition that includes a purified endophyte preparation. As used herein an “agriculturally compatible carrier” refers to any material, other than water, that can be added to a plant element without causing or having an adverse effect on the plant element (e.g., reducing seed germination) or the plant that grows from the plant element, or the like.

In some embodiments, the formulation can include a tackifier or adherent. Such agents are useful for combining the bacterial population of the invention with carriers that can contain other compounds (e.g., control agents that are not biologic), to yield a coating composition. Such compositions help create coatings around the plant or seed to maintain contact between the microbe and other agents with the plant or plant part. In some embodiments, adherents are selected from the group consisting of: alginate, gums, starches, lecithins, formononetin, polyvinyl alcohol, alkali formononetinate, hesperetin, polyvinyl acetate, cephalins, Gum Arabic, Xanthan Gum, Mineral Oil, Polyethylene Glycol (PEG), Polyvinyl pyrrolidone (PVP), Arabino-galactan, Methyl Cellulose, PEG 400, Chitosan, Polyacrylamide, Polyacrylate, Polyacrylonitrile, Glycerol, Triethylene glycol, Vinyl Acetate, Gellan Gum, Polystyrene, Polyvinyl, Carboxymethyl cellulose, Gum Ghatti, and polyoxyethylene-polyoxybutylene block copolymers.

The formulation can also contain a surfactant. Non-limiting examples of surfactants include nitrogen-surfactant blends such as Prefer 28 (Cenex), Surf-N(US), Inhance (Brandt), P-28 (Wilfarm) and Patrol (Helena); esterified seed oils include Sun-It II (AmCy), MSO (UAP), Scoil (Agsco), Hasten (Wilfarm) and Mes-100 (Drexel); and organo-silicone surfactants include Silwet L77 (UAP), Silikin (Terra), Dyne-Amie (Helena), Kinetic (Helena), Sylgard 309 (Wilbur-Ellis) and Century (Precision). In one embodiment, the surfactant is present at a concentration of between 0.01% v/v to 10% v/v. In another embodiment, the surfactant is present at a concentration of between 0.1% v/v to 1% v/v.

In certain cases, the formulation includes a microbial stabilizer. Such an agent can include a desiccant. As used herein, a “desiccant” can include any compound or mixture of compounds that can be classified as a desiccant regardless of whether the compound or compounds are used in such concentrations that they in fact have a desiccating effect on the liquid inoculant. Such desiccants are ideally compatible with the bacterial population used and should promote the ability of the microbial population to survive application on the seeds and to survive desiccation. Examples of suitable desiccants include one or more of trehalose, sucrose, glycerol, and Methylene glycol. Other suitable desiccants include, but are not limited to, non-reducing sugars and sugar alcohols (e.g., mannitol or sorbitol). The amount of desiccant introduced into the formulation can range from about 5% to about 50% by weight/volume, for example, between about 10% to about 40%, between about 15% and about 35%, or between about 20% and about 30%.

In some embodiments the formulation includes, for example, solid carriers such as talc, Fuller's earth, bentonite, kaolin clay, pyrophyllite, bentonite, montmorillonite, diatomaceous earth, acid white soil, vermiculite, and pearlite, and inorganic salts such as ammonium sulfate, ammonium phosphate, ammonium nitrate, urea, ammonium chloride, and calcium carbonate. Also, organic fine powders such as wheat flour, wheat bran, and rice bran may be used. The liquid carriers include vegetable oils such as soybean oil and cottonseed oil, glycerol, ethylene glycol, polyethylene glycol, propylene glycol, polypropylene glycol, etc.

In some embodiments, the water dispersed endophyte formulations comprises MIC-54924 in liquid fermentation broth at a minimum concentration of 1E6 CFU/g. In some embodiments, the water dispersed MIC-54924 formulation is applied as a seed treatment at a use rate of 65-130 mL/100 kg seed. The water dispersed endophyte formulations comprises MIC-54924 may be stored between 0-20C for 12 months without appreciable degradation of product efficacy.

In some embodiments, the abundance of an endophyte can be estimated by methods well known in the art including, but not limited to, qPCR, community sequencing, flow cytometry, and/or counting colony-forming units. As used herein, a “colony-forming unit” (“CFU”) is used as a measure of viable microorganisms in a sample. A CFU is an individual viable cell capable of forming on a solid medium a visible colony whose individual cells are derived by cell division from one parental cell.

In some embodiments, the synthetic composition of the present invention comprises one or more of the following: antimicrobial, fungicide, nematicide, bactericide, insecticide, or herbicide.

In some embodiments, the time to 1 log loss in CFU of an endophyte in formulations is greater than or equal to 1000 days, greater than or equal to 730 days, greater than or equal to 365 days, greater than or equal to 168 days, greater than or equal to 150 days, greater than or equal to 125 days, greater than or equal to 100 days, greater than or equal to 75 days, greater than or equal to 50 days, greater than or equal to 20 days at 4 degrees Celsius. In some embodiments, the time to 1 log loss in CFU of an endophyte in formulation is at least 1000 days, at least 730 days, at least 365 days, 140 days, at least 90 days, at least 60 days, at least 50 days, at least 30 days, at least 20 days, at 22 degrees Celsius. In some embodiments, the time to 2 log loss in CFU of an endophyte on a seed is at least 3 days, at least 5 days, at least 10 days, at least 20 days, at least 21 days, at least 22 days, at least 23 days, at least 24 days, at least 25 days, at least 30 days, at least 60 days, at least 90 days, at least 120 days at 22 degrees Celsius.

In some embodiments, a treatment is applied mechanically or manually or artificially inoculated to a plant element in a seed treatment, root wash, seedling soak, foliar application, floral application, soil inoculum, in-furrow application, sidedress application, soil pre-treatment, wound inoculation, drip tape irrigation, vector-mediation via a pollinator, injection, osmopriming, hydroponics, aquaponics, aeroponics, and combinations thereof. Application to the plant may be achieved, for example, as a powder for surface deposition onto plant leaves, as a spray to the whole plant or selected plant element, as part of a drip to the soil or the roots, or as a coating onto the plant element prior to or after planting. Such examples are meant to be illustrative and not limiting to the scope of the invention.

In some embodiments, the invention described herein provides a synthetic composition comprising one or more endophytes capable of improving plant health, wherein the one or more endophytes is a member of the Class Bacilli. In some embodiments, the one or more endophytes is a member of the Order Bacillales. In some embodiments, the one or more endophytes is a member of the Family Bacillaceae. In some embodiments, the one or more endophytes is a member of the Genus Bacillus. In some embodiments, the one or more endophytes are selected from Table 1 In some embodiments, the one or more endophytes comprise one or more polynucleotide sequences at least 95%, at least 96%, at least 97%, at least 97%, at least 98%, at least 99%, or 100% identical to one or more polynucleotide sequences selected from the group consisting of SEQ ID NOs. 5-51, or combinations thereof.

In some embodiments, the one or more endophytes comprise one or more polynucleotide sequences that are 100% identical to a subregion within one or more of SEQ ID NOs. 5-51, wherein the subregion is a 100, 200, 300, 400, 500, 600, 700, or 800 nucleotides in length.

In some embodiments, the one or more endophytes comprise one or more polynucleotide sequences that are 100% identical to a subregion within one or more of SEQ ID NOs. 6, 7, 8, 9, 10, 11, 12, 13, 14, wherein the subregion is 100 nucleotides in length. In some embodiments, the one or more endophytes comprise one or more polynucleotide sequences that are 100% identical to a subregion within one or more of SEQ ID NOs. 6, 7, 8, 9, 10, 11, 12, 13, 14, wherein the subregion is 200 nucleotides in length. In some embodiments, the one or more endophytes comprise one or more polynucleotide sequences that are 100% identical to a subregion within one or more of SEQ ID NOs. 6, 7, 8, 9, 10, 11, 12, 13, 14, wherein the subregion is 400 nucleotides in length. In some embodiments, the one or more endophytes comprise one or more polynucleotide sequences that are 100% identical to a subregion within one or more of SEQ ID NOs. 6, 7, 8, 9, 10, 11, 12, 13, wherein the subregion is 600 nucleotides in length.

In some embodiments, the one or more endophytes comprise one or more polynucleotide sequences that are 100% identical to a subregion within one or more of SEQ ID NOs. 15, 16, 17, 18, 19, 20, 21, 22, wherein the subregion is 100 nucleotides in length. In some embodiments, the one or more endophytes comprise one or more polynucleotide sequences that are 100% identical to a subregion within one or more of SEQ ID NOs. 15, 16, 17, 18, 19, 20, 21, 22, wherein the subregion is 200 nucleotides in length. In some embodiments, the one or more endophytes comprise one or more polynucleotide sequences that are 100% identical to a subregion within one or more of SEQ ID NOs. 15, 16, 17, 18, 19, 20, 21, 22, wherein the subregion is 400 nucleotides in length. In some embodiments, the one or more endophytes comprise one or more polynucleotide sequences that are 100% identical to a subregion within one or more of SEQ ID NOs. 16, 17, 18, 19, 21, 22, wherein the subregion is 600 nucleotides in length.

In some embodiments, the one or more endophytes comprise one or more polynucleotide sequences that are 100% identical to a subregion within one or more of SEQ ID NOs. 23, 24, 25, 26, 27, wherein the subregion is 100 nucleotides in length. In some embodiments, the one or more endophytes comprise one or more polynucleotide sequences that are 100% identical to a subregion within one or more of SEQ ID NOs. 23, 24, 25, 26, 27, wherein the subregion is 200 nucleotides in length. In some embodiments, the one or more endophytes comprise one or more polynucleotide sequences that are 100% identical to a subregion within one or more of SEQ ID NOs. 23, 24, 25, 26, 27, wherein the subregion is 400 nucleotides in length. In some embodiments, the one or more endophytes comprise one or more polynucleotide sequences that are 100% identical to a subregion within one or more of SEQ ID NOs. 23, 24, wherein the subregion is 600 nucleotides in length.

In some embodiments, the one or more endophytes comprise one or more polynucleotide sequences that are 100% identical to a subregion within one or more of SEQ ID NOs. 28, 29, 30, 31, 32, 33, 34, 35, 36, 37, 38, 39, wherein the subregion is 100 nucleotides in length. In some embodiments, the one or more endophytes comprise one or more polynucleotide sequences that are 100% identical to a subregion within one or more of SEQ ID NOs. 28, 29, 30, 31, 32, 33, 34, 35, 36, 37, 38, 39, wherein the subregion is 200 nucleotides in length. In some embodiments, the one or more endophytes comprise one or more polynucleotide sequences that are 100% identical to a subregion within one or more of SEQ ID NOs. 28, 29, 30, 31, 35, 36, 37, 38, 39, wherein the subregion is 400 nucleotides in length. In some embodiments, the one or more endophytes comprise one or more polynucleotide sequences that are 100% identical to a subregion within one or more of SEQ ID NOs. 28, 29, 30, 31, 35, 36, 37, 38, 39, wherein the subregion is 600 nucleotides in length.

In some embodiments, the one or more endophytes comprise one or more polynucleotide sequences that are 100% identical to a subregion within one or more of SEQ ID NOs. 42, 43, 44, 45, 46, 47, 48, 49, 50, 51, wherein the subregion is 100 nucleotides in length. In some embodiments, the one or more endophytes comprise one or more polynucleotide sequences that are 100% identical to a subregion within one or more of SEQ ID NOs. 42, 43, 44, 45, 46, 47, 48, 49, 50, 51, the subregion is 200 nucleotides in length. In some embodiments, the one or more endophytes comprise one or more polynucleotide sequences that are 100% identical to a subregion within one or more of SEQ ID NOs. 42, 43, 44, 46, 48, 49, wherein the subregion is 400 nucleotides in length. In some embodiments, the one or more endophytes comprise one or more polynucleotide sequences that are 100% identical to a subregion within one or more of SEQ ID NOs. 42, 43, 44, 48, wherein the subregion is 600 nucleotides in length.

In some embodiments, the one or more endophytes comprise one or more polynucleotide sequences that are 100% identical to a subregion within SEQ ID NO. 40, wherein the subregion is 100 nucleotides in length. In some embodiments, the one or more endophytes comprise one or more polynucleotide sequences that are 100% identical to a subregion within SEQ ID NO. 40, wherein the subregion is 200 nucleotides in length.

In some embodiments, the one or more endophytes comprise one or more polynucleotide sequences that are 100% identical to a subregion within SEQ ID NO. 41, wherein the subregion is 100 nucleotides in length. In some embodiments, the one or more endophytes comprise one or more polynucleotide sequences that are 100% identical to a subregion within SEQ ID NO. 41, wherein the subregion is 200 nucleotides in length. In some embodiments, the one or more endophytes comprise one or more polynucleotide sequences that are 100% identical to a subregion within SEQ ID NO. 41, wherein the subregion is 400 nucleotides in length. In some embodiments, the one or more endophytes comprise one or more polynucleotide sequences that are 100% identical to a subregion within SEQ ID NO. 41, wherein the subregion is 600 nucleotides in length.

The amino acid variants of MIC-54924 W1 listed in Table 22 which may be engineered and expressed in any protein expression system. For example, a protein expression system may be E. coli and isolated, recombinantly produced W1 of SEQ ID 100-134 heterologously applied to the exterior of a plant element. As an additional example, an endophyte expressing a protein having amino acid sequence of one or more of W1 of SEQ ID 100-134 may be applied to a plant element.

In some embodiments of any of the synthetic compositions described herein, the synthetic compositions comprise at least 2, 3, 4, 5, 6, 7, 8, 9, 10, 15 or 20 or more endophytes. In some embodiments, the one or more endophytes comprise at least 2, 3, 4, 5, 6, 7, 8, 9, 10, 15 or 20 or more endophytes. In some embodiments, the one or more endophytes are distinct individual organisms or distinct members of different genetic derivation or taxa.

### Methods for Improving Plant Health

In some embodiments, the invention provides methods of improving plant health comprising heterologously disposing one or more endophytes to a plant element in an effective amount to increase a trait of agronomic importance in the plant derived from the treated plant element relative to a plant derived from a reference plant element. In some embodiments, the one or more endophytes are a component of a treatment formulation. In some embodiments, the one or more endophytes are a component of a synthetic composition.

In some embodiments, the invention provides methods of improving plant health comprising creating any of the synthetic compositions described herein, wherein the synthetic composition comprises any of the plant elements of any of the plants described herein and any of the one or more endophytes described herein. In some embodiments, the synthetic composition comprises any of the treatment formulations described herein and any of the one or more endophytes described herein. In some embodiments, the synthetic composition additionally comprises a growth medium or growth environment. A growth environment is a natural or artificially constructed surrounding, capable of supporting the life of a plant. In some embodiments, the growth medium is soil. In some embodiments, the growth medium is a culture fluid suitable for propagation of an endophyte or plant tissue culture. In some embodiments, the method comprises a step of applying the synthetic composition to a growth medium. In some embodiments, the synthetic composition is applied before one or more plant elements are placed in or on the growth medium. In some embodiments, the synthetic composition is applied after one or more plant elements are placed in or on the growth medium. In some embodiments, the method comprises a step of germinating the plants. In some embodiments, the method comprises a step of growing the plants. For example, the plants may be grown in the plant vigor assays, greenhouse assessment, and or field trials described herein. In some embodiments, the method comprises a step of growing the plants to maturity. In some embodiments, where the plants are commercially produced, maturity is the stage at which the plant is normally harvested.

In some embodiments of any of the methods described herein, plant health may be improved for plants in a stress condition. In some embodiments, the stress condition is a biotic or abiotic stress, or a combination of one or more biotic or abiotic stresses. In some embodiments of any of the methods described herein, the stress condition is one or more of the following abiotic stresses: drought stress, salt stress, metal stress, heat stress, cold stress, low nutrient stress (alternately referred to herein as nutrient deficiency or growth in nutrient deficient conditions), and excess water stress, and combinations thereof. In some embodiments of any of the methods described herein, the stress condition is one or more of the following biotic stresses: insect infestation, nematode infestation, complex infection, fungal infection, bacterial infection, oomycete infection, protozoal infection, viral infection, herbivore grazing, and combinations thereof. In some embodiments, the biotic stress condition is the presence of one or more of soybean cyst nematode (e.g. Heterodera glycines), root-knot nematode (e.g. Meloidogyne incognita), root lesion nematode (e.g. Pratylenchus brachyurus), cyst nematode (e.g. Heterodera and Globodera spp.), dagger nematode (e.g. Xiphinema spp.), lance nematode (e.g. Hoplolaimus galeatus), lesion nematode (e.g. Pratylenchus spp.), needle nematode (e.g. Longidorus spp.), reniform nematode (e.g. Rotylenchulus reniformis), spiral nematode (e.g. Helicotylenchus spp.), sting nematode (e.g. Belonolaimus longicaudatus), stubby-root nematode (e.g. Trichodorus and Paratrichodorus spp.), and stunt nematode (e.g. Tylenchorhynchus spp., Anguillulina spp., and Merlinia) spp.).

Stress tolerance is exemplified by improvement of one or more other traits of agronomic importance when compared with a reference plant, reference plant element, or reference plant population. For example, biotic stress tolerance may be shown by one or more of decreased pathogen load of tissues, decreased area of chlorotic tissue, decreased necrosis, improved growth, increased survival, increased biomass, increased shoot height, increased root length, relative to a reference.

### Methods for Measuring Plant Health

The present invention includes methods of measuring plant health, comprising determining the presence or abundance of one or more endophytes in a plant element, growth medium, and or growth environment. In some embodiments, the abundance or presence of the one or more endophytes in a plant element in an effective amount to improve a trait of agronomic importance is an indicator of plant health. In some embodiments, the abundance or presence of the one or more endophytes in a growth medium and or growth environment in an effective amount to improve a trait of agronomic importance of a plant element grown in the growth environment or growth medium may be used as a measure or predictor of plant health in a plant grown in that growth environment or growth medium. In some embodiments, the presence or abundance of one or more endophytes in a plant element, growth medium or growth environment can be detected before an improvement of a trait of agronomic importance can otherwise be observed or detected. In some embodiments, the presence or abundance of one or more endophytes is determined by polymerase chain reaction, fluorescence in situ hybridization, or isothermal amplification.

### Nucleic Acid Probes and Detection Kits

The present invention includes one or more nucleic acid probes that are markers of improved plant health. These probes include single and double stranded nucleic acids, engineered polymers such as peptide nucleic acids, or combinations thereof. In some embodiments, there are a plurality of nucleic acid probes. In some embodiments, the nucleic acid probes are attached to one or more solid supports. In some embodiments, the nucleic acid probes are reversibly attached to one or more solid supports. In some embodiments, the nucleic acid probes are attached to a contiguous solid support. In some embodiments, the nucleic acid probes are attached to a plurality of particles, for example beads. In some embodiments, only one unique sequence is attached to each particle. In some embodiments, nucleic acid probes attached to a solid support are physically separated from non-identical probes by an indentation or raised portion of the solid support. In some embodiments, the invention described herein provides a nucleic acid detection kit comprising any of the plurality of nucleic acid probes described herein.

In some embodiments, the one or more nucleic acid probes of the present invention may comprise sequences complementary or reverse complementary to one or more polynucleotide sequences selected from the group consisting of SEQ ID NOs. 5-51, or combinations thereof. In some embodiments, the one or more nucleic acid probes of the present invention may comprise nucleic acid sequences complementary or reverse complementary to a nucleic acid sequence that is at least 97% identical to one or more polynucleotide sequences selected from the group consisting of SEQ ID NOs. 5-51, or combinations thereof. In some embodiments, the one or more nucleic acid probes of the present invention may comprise sequences complementary or reverse complementary to the entire length of one or more polynucleotide sequences selected from the group consisting of SEQ ID NOs. 5, or combinations thereof. In some embodiments, the one or more nucleic acid probes of the present invention may comprise sequences complementary or reverse complementary to a region within one or more polynucleotide sequences selected from the group consisting of SEQ ID NOs.-51, or combinations thereof. In some embodiments, the region to which the nucleic acid probe is complementary or reverse complementary is a contiguous region. In some embodiments, the region to which the nucleic acid probe is complementary or reverse complementary is at least 5 nucleotides (nt) in length, at least 10 nt in length, at least 15 nt, between 10 nt and 30 nt, between 10 and 20 nt, between 15 and 50 nt, at least 20 nt, between 20 and 60 nt, at least 25 nt, at least 30 nt, at least 40 nt, at least 50 nt, between 50 nt and 100 nt, at least 60 nt, at least 70 nt, at least 80 nt, at least 100 nt in length. In some embodiments, the regions to which the nucleic acid probe is complementary or reverse complementary is not a contiguous region.

In some embodiments, a nucleic acid probe is capable of hybridizing to one or more polynucleotide sequences selected from the group consisting of SEQ ID NOs. 5-51, or combinations thereof, or a reverse complement thereof. In some embodiments, the nucleic acid probe is capable of hybridizing under moderate conditions. “Moderate conditions” are 0.165M-0.330M NaCl and 20-29 degrees Celsius below the melting temperature of the nucleic acid probe. In some embodiments, the nucleic acid probe is capable of hybridizing under stringent conditions. “Stringent conditions” are 0.0165M-0.0330M NaCl and 5-10 degrees Celsius below the melting temperature of the nucleic acid probe.

In some embodiments, the nucleic acid probes are a component of a nucleic acid detection kit. In some embodiments, the nucleic acid probes are a component of a DNA detection kit. In some embodiments, the nucleic acid detection kit comprises additional reagents. In some embodiments, the contents of the nucleic acid detection kit are utilized in performing DNA sequencing.

In some embodiments, the one or more nucleic acid probes comprises at least 2, 3, 4, 5, 6, 7, 8, 9, or 10 probes.

The invention will be further described in the following examples, which do not limit the scope of the invention described in the claims.

### EXAMPLES

### Example 1. Isolation and Identification of Endophytes

Endophytes of the present invention were isolated as described in Table 1 and Table 2.

TABLE 1
Sources of microbes of the present invention

| MICID | Taxonomy | Isolated From | Isolation Environment |
|---|---|---|---|
| MIC-54924 | Bacillus | Glycine max | Cold |
| | aryabhattai | | |

TABLE 2
Method of isolating microbes of the present invention

| MIC | |
|---|---|
| ID | Isolation Method |
| 54924 | Healthy, whole, field-grown seedlings of Glycine max (soybean) were collected from an |
| MIC- | agricultural field in Indiana, USA. Plant tissue samples were washed in a dilute water and detergent |
| | solution and were surface sterilized by successive rinses: 2 minutes in 10% bleach solution, 2 |
| | minutes in 70% ethanol solution, and a series of three rinses with sterile water. The plant tissue was |
| | cut into small pieces with sterile scissors and blended with 3, 7 mm steel beads in 5-7.5 ml |
| | phosphate buffered solution (PBS). Serial dilutions of tissue homogenate were plated on a panel of |
| | media types for endophyte cultivation. A non-descript, non-pigmented colony arose on Nutrient |
| | Agar after 4 days of incubation at 24° C. and was streaked to purity and stored in glycerol (20% v/v) |
| | at −80° C. until subjected to further testing. |

### Phylogenetic and Genomic Analysis of Endophytes

Phylogenetic and genomic analyses for bacterial strains. According to the manufacturer's protocol, DNA was extracted from pure cultures using the Omega Mag-Bind Universal Pathogen Kit with a final elution volume of 60 μl (Omega Biotek Inc., Norcross, GA). DNA samples were quantified using a Qubit fluorometer (ThermoFisher Scientific, Waltham, MA) and normalized to 100 ng. DNA was prepared using the Nextera DNA Flex Library Prep Kit according to the manufacturer's instructions (Illumina Inc., San Diego, CA). DNA libraries were quantified via qPCR using the KAPA Library Quantification kit (Roche Sequencing and Life Science, Wilmington, MA) and combined in equimolar concentrations into one 24-sample pool. Libraries were sequenced on a MiSeq using pair-end reads (2×200 bp). Reads were trimmed of adapters and low-quality bases using Cutadapt (version 1.9.1) and assembled into contigs using MEGAHIT (version 1.1.2) (Li, D., Liu, C.-M., Luo, R., Sedaxane, K., and Lam, T.-W. 2015. MEGAHIT: an ultra-fast single-node solution for large and complex metagenomics assembly via succinct de Bruijn graph. Bioinformatics. 31:1674-1676). Reads were mapped to contigs using Bowtie2 (version 2.3.4) (Langmead, B., and Salzberg, S. L. 2012. Fast gapped-read alignment with bowtie 2. Nat Methods. 9 Available at: doi.org/10.1038/nmeth.1923.), and contigs were assembled into scaffolds using BESST (2.2.8) (Sahlin, K., Vezzi, F., Nystedt, B., Lundeberg, J., and Arvestad, L. 2014. BESST-efficient scaffolding of large fragmented assemblies. BMC bioinformatics. 15:281).

Genes for phylogenetic analyses were extracted from genome assemblies using barrnap (Seemann, T. 2019. barrnap 0.9: rapid ribosomal RNA prediction. Available at: github.com/tseemann/barrnap) or blast (Altschul, S. F., Madden, T. L., Schäffer, A. A., Zhang, J., Zhang, Z., Miller, W., et al. 1997. Gapped BLAST and PSI-BLAST: A new generation of protein database search programs. Nucleic Acids Research. 25:3389-3402). Homologous DNA sequences from type strains or other, likely correctly identified strains were retrieved from GenBank and aligned using MAFFT (Katoh, K., and Standley, D. M. 2013. MAFFT multiple sequence alignment software version 7: improvements in performance and usability. Molecular Biology and Evolution. 30:772-780), or other software. Single or multilocus phylogenetic analyses were performed using PAUP (Swofford, D. L. 2002. PAUP*. Phylogenetic Analysis Using Parsimony (*and Other Methods). Version 4. Sunderland, Massachusetts: Sinauer Associates) or similar software.

16S rRNA gene sequences were extracted from genome assemblies using barrnap (Seemann 2019). Phylogenomic analyses were performed using GToTree (Lee, M. D. 2019. Applications and considerations of GToTree: a user-friendly workflow for phylogenomics. Evolutionary Bioinformatics. 15:1176934319862245) with default settings. Average nucleotide identity analyses were performed using the pyani ANIm algorithm (Richter, M., and Rosselló-Móra, R. 2009. Shifting the genomic gold standard for the prokaryotic species definition. Proceedings of the National Academy of Sciences. 106:19126-19131) implemented in the MUMmer package (Kurtz, S., Phillippy, A., Delcher, A. L., Smoot, M., Shumway, M., Antonescu, C., et al. 2004. Versatile and open software for comparing large genomes. Genome biology. 5: R12) retrieved from github.com/widdowquinn/pyani.

Identification of bacterial strains. A bacteria is identified at the species level, if its average nucleotide identity (ANI) was >95% to the genome of a single species represented by its type strain downloaded from GenBank. Phylogenomic analyses were also performed if a bacteria had >1 species with >95% ANI, or the gap between the top two ANI hits was <3%, in this case, the bacteria is identified at the genus and species if it had a single sister group with >70% bootstrap support.

Genomic analysis methods. Key genomic features were identified using standard bioinformatic analysis including BLAST search, presence of known protein domains within the translated gene product, homology to members of protein families, and homology to functional orthologs. The presence of several genomic features in MIC-54924 make plant beneficial bacteria capable of improving plant health and productivity under biotic stresses. The features can be grouped in two, capability to induce plant's defense and antagonism mediated by lytic enzymes and putative effectors.

The genome of MIC-54924 contains multiple features that support a plant associated lifestyle. Its capabilities to biocontrol activities, to use plant derived photosynthates, and to store nutrients can provide it competitive advantage. The genome of MIC-54924 contains genetic capabilities to promote growth as well as benefit plant by mobilizing soil nutrients.

The genome of MIC-54924 contains multiple features that support nutrient acquisition, including: presence of assimilatory nitrate reduction enzymes (nasBCDE), nitrate, nitrite and ammonia transports (nark, nrgA, amtB), and assimilation pathways; TnrA, a transcription regulator under N-limitation; urease and accessory proteins (ureABCDEFG) to catabolize urea producing ammonia; acid and alkaline phosphatases (phoABDEHPRU) to access organically bound phosphate nutrients; degradation of organophosphonates (phn genes); mobilize soil macro and micronutrients for plant uptake-presence of transporter genes for K, P, S, Mg [kdpAB, kimA (8 copies), ktrBC, pit, pstABS]; Fe-siderophores binding proteins and transporters for iron acquisition.

The genome of MIC-54924 contains multiple features that support plant growth, and stress resilience, including: biosynthesis of 2,3-butanediol and acetoin [bdhA, acuABC, alsS (2 copies)], these VOCs can induce plant ISR and promote growth; putative auxin biosynthesis (yhcX, dhaS, patB, bsdBC, ysnE) and auxin efflux carrier (ywkB, homologous to auxin-specific PIN transporter of plant) genes; Cytokinins (miaAB, 3 copies of LOG protein), Spermidine (speABCEGHE) and Riboflavin (rib genes) biosynthesis to promote plant growth; presence of osmoprotectant transport system (opu genes); osmolytes (glycine betaine, GABA, proline, trehalose) biosynthesis and transport; multiple water channels for osmotic balance; ROS scavenging enzymes (cat1, katAEGX, sodACF, ydhU) to reduce oxidative stress; biosynthesis of antioxidant carotenoid; over 35 different genes coding for stress proteins (csp, hsp etc.).

The genome of MIC-54924 contains multiple features that support niche establishment, including: flagellar components (flg and fli genes) and motility proteins (motAB); S-ribosylhomocysteine lyase (luxS) for quorum sensing; genes for capsular polysaccharides synthesis and chemotaxis, Swarming motility proteins (swrC); biofilm biosynthetic/capsule synthesis and regulatory genes and extracellular glycolytic enzymes and proteases; presence of genes encoding chitin deacetylases and chitinase (chia); detected in a gene encoding a putative insecticidal neutral metalloprotease.

The genome of MIC-54924 contains multiple features that support plant associated lifestyle, including: sucrose degradation III (sucrose invertase 3 copies) pathway; presence of genes coding for glucoamylases, glucan hydrolases and alpha amylases; presence of Levansucrase and associated genes (sacB_1, sacB_2, sacPT), levanbiose-producing levanase, associated genes (levBDEFGR) and fructose transporters for efficient colonization of sucrose-rich habitats; Trehalose utilization thuA and trehalose degradation I and transport (low osmolarity) (treAPR); phenolic acid decarboxylase (padCR, bsdCD) to detoxify plant produced antimicrobials; presence of Ankyrin repeat domains; presence of branched amino acid transporters (6); transporters for dicarboxylate (4 dct genes). Exemplary genomic features and protein products of are listed in Table 4.

### Identification of Endophytes by Sequencing of Marker Genes

The endophytes were characterized by the sequences of genomic regions. Primers that amplify genomic regions of the endophytes of the present invention are listed in Table 3. Sanger sequencing was performed at Genewiz (South Plainfield, NJ). Raw chromatograms were converted to sequences, and corresponding quality scores were assigned using TraceTuner v3.0.6beta (U.S. Pat. No. 6,681,186). These sequences were quality filtered, aligned and a consensus sequence generated using Geneious v 8.1.8 (Biomatters Limited, Auckland NZ).

TABLE 3
Primer sequences useful in identifying microbes of the present
invention

| SeqID | Primer | Description | Sequence |
|---|---|---|---|
| 1 | 27f | 16S ribosomal RNA | AGAGTTTGATYMTGGCTCAG |
| 2 | 1492r | 16S ribosomal RNA | GGTTACCTTGTTACGACTT |
| 3 | 515f | 16S ribosomal RNA | GTGYCAGCMGCCGCGGTAA |
| 4 | 806r | 16S ribosomal RNA | GGACTACNVGGGTWTCTAAT |

MIC-54924 was deposited with ______ as Deposit ID ______.

TABLE 4
Genomic features and protein products.

| | | Locus | |
|---|---|---|---|
| Sequence Category | SEQ ID | Abbreviation | Sequence Description |
| 2,3-butanediol and | 11, 57 | bdhA | (R,R)-butanediol dehydrogenase |
| acetoin biosynthesis | 6, 52 | acuA | Acetoin utilization protein AcuA |
| | 7, 53 | acuB | Acetoin utilization protein AcuB |
| | 8, 54 | acuC | Acetoin utilization protein AcuC |
| | 9, 55 | alsS_1 | Acetolactate synthase |
| | 10, 56 | alsS_2 | Acetolactate synthase |
| | 13, 59 | ilvB | acetolactate synthase, large subunit, biosynthetic type |
| | 14, 60 | ilvN | acetolactate synthase, small subunit |
| | 12, 58 | budA | Alpha-acetolactate decarboxylase |
| Antifungal peptide | 40, 97 | yrbF | Sec translocon accessory complex subunit YrbF, protien |
| W1 | | | cleaved to produce antifungal peptide W1 |
| | 98, 99 | W1 | antifungal peptide W1 |
| Auxin biosynthesis | 19, 65 | yhcX | Hydrolase YhcX |
| and transport | 18, 64 | patB | Patbp |
| | 16, 62 | bsdC | Phenolic acid decarboxylase |
| | 17, 63 | dhaS | Putative aldehyde dehydrogenase DhaS |
| | 20, 66 | ysnE | putative N-acetyltransferase YsnE |
| | 21, 67 | ywkB_1 | putative transporter YwkB |
| | 22, 68 | ywkB_2 | putative transporter YwkB |
| | 15, 61 | bsdB | putative UbiX-like flavin prenyltransferase |
| Cytokinin | 25, 71 | yvdD_1 | Belongs to the LOG |
| biosynthetic genes | 26, 72 | yvdD_2 | Belongs to the LOG |
| | 27, 73 | yvdD_3 | Belongs to the LOG |
| | 23, 69 | miaA | tRNA isopentenyltransferase |
| | 24, 70 | miaB | tRNA-2-methylthio-N(6)-dimethylallyladenosine synthase |
| Fungal | 28, 74 | bglA | Aryl-phospho-beta-D-glucosidase BglA |
| polysaccharides, | 29, 75 | bglH5 | Belongs to the glycosyl hydrolase 1 |
| chitin and lichenan, | 33, 79 | licB_1 | Beta-glucanase |
| degradation & | 34, 80 | licB_2 | Beta-glucanase |
| transport genes | 30, 76 | cdal | Chitin deacetylase 1 |
| | 31, 77 | Chia | Glyco 18, chitinase |
| | 35, 81 | licC | Lichenan permease IIC component |
| | 32, 78 | licA | Lichenan-specific phosphotransferase enzyme IIA |
| | | | component, lichenan transport |
| | 39, 85 | licR | Mga helix-turn-helix domain, transcription regulator |
| | 36, 82 | licH_1 | putative 6-phospho-beta-glucosidase |
| | 37, 83 | licH_2 | putative 6-phospho-beta-glucosidase |
| | 38, 84 | licH_3 | putative 6-phospho-beta-glucosidase |
| Putative protease | 41, 86 | ina | Immune inhibitor A, Neutral metalloprotease with |
| with biocontrol | | | biocontrol activity |
| activity | | | |
| Type Secretion | 49, 94 | yueC | ESX secretion system protein YueC |
| Systems VII & | 42, 87 | bsn_1 | Extracellular ribonuclease, putative effector |
| effectors for | 51, 96 | yukE | Protein YukE, type VII secretion systems (T7SS) effector |
| microbial | | | for iron acquisition |
| competitiveness | 46, 91 | 000164 | putatative effector of Type VII secretion system |
| | 47, 92 | 000165 | putatative effector of Type VII secretion system |
| | 45, 90 | 000163 | putatative effector of Type VII secretion system, |
| | | | ribonuclease |
| | 44, 89 | essC | Type VII secretion protein EssC |
| | 48, 93 | yueB | Type VII secretion system EsaA |
| | 43, 88 | essB | WXG100 protein secretion system (Wss), protein YukC |
| | 50, 95 | yukD | WXG100 protein secretion system (Wss), protein YukD; |
| | | | EsaB |

### Example 2. Production of Microbial Treatments

Preparation of bacterial endophyte treatments: A sterile loop is used to inoculate each bacteria by taking a looping into broth using a transfer tube into 3 mL tryptic soy broth in a 24-well plate. A breathable cover is placed over the 24-well plate. The plate is incubated at room temperature at 400 rpm on a shaker for 3 days. At the end of the incubation period, the 24-well plate containing cultures is centrifuged at 5000 rpm for 4 minutes. The supernatant is removed from the opposite edge of tube leaving the pellet at the bottom of the tube. The pellet should be free of any culture filtrate. The pellet is resuspended in 2-3 mL of 1×PBS solution and mixed well. A 1:2 dilution for OD reading is made by transferring 50 μl of resuspended pellet into a clear bottom 96-well OD reading plate with 50 μl 1×PBS to achieve a final volume of 100 μl. An appropriate blank is made (50 μl 1xPBS, 50 μl uninoculated broth), and the OD600 read. The concentration of each culture is adjusted by diluting in PBS to 0.05 OD. The cultures are plated to check for colony forming units.”

Additional methods of producing endophyte treatments include the following methods.

An agar plug of each bacterial strain is transferred using a transfer tube to 4 ml of potato dextrose broth (PDB) in a 24-well plate and incubated at room temperature at 675 rpm on a shaker for 3 days. After growth of bacteria in broth, 200 μl is transferred into a spectrophotometer reading plate and bacteria OD is read at 600 nm absorbance. All bacteria strains are then normalized to the desired concentration using PBS 1x buffer.

### Example 3. Formulation of Endophyte Treatments

Method of Treating Seeds with Wettable Powder Formulations

Wettable powder endophyte formulations comprise endophyte biomass, a clay carrier, sugar, protein, dispersant, and/or surfactant. The volume of seeds was used to determine the volume of endophyte slurry needed for the target dose per seed, where the total slurry comprises 95% water and 5% wettable powder. The calculated volume of water was added to the mix tank, and the endophyte in wettable powder was added to a clean mix tank. The contents of the tank were mixed for five minutes to ensure the powder was well dispersed in the tank. Agitation was maintained in the mix tank during seed treatment to limit settling of the product. The required volume of slurry was then applied to the seeds and the seeds were gently mixed until the slurry was evenly dispersed.

Method of Treating Seeds with Water Dispersed Formulations

Water dispersed endophyte formulations comprise endophyte biomass in liquid fermentation broth that may be diluted in a buffered carrier such as phosphate buffered saline as well as a preservative and/or a pH adjusting agent. The volume of seeds was used to determine the volume of endophyte in water dispersion formulation needed for the target dose per seed. The calculated volume of endophyte formulation was added to the seeds in a clean mixing vessel. The seeds and endophyte formulation were mixed for at least 30 seconds to ensure the endophyte formulation was well dispersed on the seeds.

Method of Treating Seeds with Oil Dispersed Formulations

Oil dispersion formulations comprise endophyte biomass, a vegetable oil-based carrier, a dispersant, and/or a rheology modifier. The volume of seeds is used to determine the volume of endophyte in oil dispersion formulation needed for the target dose per seed. The oil dispersed endophyte formulation is thoroughly agitated to resuspend the endophyte throughout the formulation. The calculated volume of endophyte formulation is added to the seeds in a clean mixing vessel. The seeds and endophyte formulation are mixed to ensure the endophyte formulation was well dispersed on the seeds.

Method of Treating Seeds with Flowable Powder Formulations

Flowable powder endophyte formulations comprise talc, mineral oil base, desiccant (optionally), and spray dried or solid state fermentation produced endophyte. The volume of seeds was used to determine the volume of endophyte in a flowable powder formulation needed for the target dose per seed. The seeds to be treated were added to a clean mixing vessel. The calculated volume of endophyte formulation for the desired dose was added to the seeds in a clean mixing vessel. The seeds and endophyte formulation were mixed for at least 30 seconds to ensure the endophyte formulation was well dispersed on the seeds.

### Example 4. Additional Methods for Creating Synthetic Compositions

### Osmopriming and Hydropriming

One or more endophytes are inoculated onto seeds during the osmopriming (soaking in polyethylene glycol solution to create a range of osmotic potentials) and/or hydropriming (soaking in de-chlorinated water) process. Osmoprimed seeds are soaked in a polyethylene glycol solution containing one or more endophytes for one to eight days and then air dried for one to two days. Hydroprimed seeds are soaked in water for one to eight days containing one or more endophytes and maintained under constant aeration to maintain a suitable dissolved oxygen content of the suspension until removal and air drying for one to two days. Talc and or flowability polymer are added during the drying process.

### Foliar Application

One or more endophytes are inoculated onto aboveground plant tissue (leaves and stems) as a liquid suspension in dechlorinated water containing adjuvants, sticker-spreaders and UV protectants. The suspension is sprayed onto crops with a boom or other appropriate sprayer.

### Soil Inoculation

One or more endophytes are inoculated onto soils in the form of a liquid suspension, either; pre-planting as a soil drench, during planting as an in-furrow application, or during crop growth as a side-dress. One or more endophytes are mixed directly into a fertigation system via drip tape, center pivot or other appropriate irrigation system.

Hydroponic and Aeroponic inoculation

One or more endophytes are inoculated into a hydroponic or aeroponic system either as a powder or liquid suspension applied directly to the rockwool substrate or applied to the circulating or sprayed nutrient solution.

### Vector-Mediated Inoculation

One or more endophytes are introduced in powder form in a mixture containing talc or other bulking agent to the entrance of a beehive (in the case of bee-mediation) or near the nest of another pollinator (in the case of other insects or birds). The pollinators pick up the powder when exiting the hive and deposit the inoculum directly onto the crop's flowers during the pollination process.

### Root Wash

The method includes contacting the exterior surface of a plant's roots with a liquid inoculant formulation containing one or more endophytes. The plant's roots are briefly passed through standing liquid microbial formulation or liquid formulation is liberally sprayed over the roots, resulting in both physical removal of soil and microbial debris from the plant roots, as well as inoculation with microbes in the formulation.

### Seedling Soak

The method includes contacting the exterior surfaces of a seedling with a liquid inoculant formulation containing one or more endophytes. The entire seedling is immersed in standing liquid microbial formulation for at least 30 seconds, resulting in both physical removal of soil and microbial debris from the plant roots, as well as inoculation of all plant surfaces with microbes in the formulation. Alternatively, the seedling can be germinated from seed in or transplanted into media soaked with the microbe(s) of interest and then allowed to grow in the media, resulting in soaking of the plantlet in microbial formulation for much greater time, for example: hours, days, or weeks. Endophytic microbes likely need time to colonize and enter the plant, as they explore the plant surface for cracks or wounds to enter, so the longer the soak, the more likely the microbes will successfully be installed in the plant.

### Wound Inoculation

The method includes contacting the wounded surface of a plant with a liquid or solid inoculant formulation containing one or more endophytes. Plant surfaces are designed to block entry of microbes into the endosphere, since pathogens attempt to infect plants in this way. One way to introduce beneficial endophytic microbes into plant endospheres is to provide a passage to the plant interior by wounding. This wound can take a number of forms, including pruned roots, pruned branches, puncture wounds in the stem breaching the bark and cortex, puncture wounds in the tap root, puncture wounds in leaves, puncture wounds in the seed allowing entry past the seed coat. Wounds can be made using tools for physical penetration of plant tissue such as needles. Microwounds may also be introduced by sonication. The microbial inoculant, as liquid, as powder, inside gelatin capsules, in a pressurized capsule injection system, or in a pressurized reservoir and tubing injection system, can then be contacted into the wound, allowing entry and colonization by microbes into the endosphere. Alternatively, the entire wounded plant can be soaked or washed in the microbial inoculant for at least 30 seconds, giving more microbes a chance to enter the wound, as well as inoculating other plant surfaces with microbes in the formulation—for example pruning seedling roots and soaking them in inoculant before transplanting is a very effective way to introduce endophytes into the plant.

### Injection

The method includes injecting microbes into a plant to successfully install them in the endosphere. Plant surfaces are designed to block entry of microbes into the endosphere, since pathogens attempt to infect plants in this way. To introduce beneficial endophytic microbes to endospheres, we need a way to access the interior of the plant which we can do by puncturing the plant surface with a needle and injecting microbes into the inside of the plant. Different parts of the plant can be inoculated this way including the main stem or trunk, branches, tap roots, seminal roots, buttress roots, and even leaves. The injection can be made with a manual, mechanical, or biological injection system, and through the puncture wound can then be contacted the microbial inoculant as liquid, as powder, inside gelatin capsules, in a pressurized capsule injection system, or in a pressurized reservoir and tubing injection system, allowing entry and colonization by microbes into the endosphere.

### Example 5. Viability Over Time of Endophytes in Synthetic Fertilizer Compositions

This example describes an exemplary method by which compatibility of synthetic compositions comprising endophytes and fertilizers may be evaluated.

Application rates. Fertilizer compositions may be granular or liquid in form and comprise nitrogen, phosphorous, sulfur, zinc, micronutrients, urease inhibitors, monoammonium phosphate (MAP), and/or triple superphosphate (TSP). Flowable powder (FP) endophyte treatments, prepared as described above, have a target application rate of 3.6 grams per acre. Water dispersal (WD) endophyte treatments, prepared as described above, have a target application rate of 13 grams per acre. Synthetic compositions are prepared using concentrations of endophyte and fertilizer (% w/w), representing between 5-50 times the target application rate. Synthetic compositions are blended and stored at either 22° C. with between 20-60% relative humidity or 30° C. with 80% relative humidity. The endophytes are reisolated from the synthetic compositions or seeds, on the day of treatment and each following period and the CFU recorded.

### Example 6. Assessment of Improved Plant Characteristics: Vigor Assay

### Assay of Soybean Seedling Vigor

Seed preparation: The lot quality of soybean seeds is first assessed by testing germination of 100 seeds. Seeds are placed, 8 seeds per petri dish, on filter paper in petri dishes, 12 ml of water is added to each plate and plates are incubated for 3 days at 24° C. The process should be repeated with a fresh seed lot if fewer than 95% of the seeds have germinated. One thousand soybean seeds are then surface sterilized by co-incubation with chlorine gas in a 20×30 cm container placed in a chemical fume hood for 16 hours. Percent germination of 50 seeds, per sterilization batch, is tested as above and confirmed to be greater than 95%.

Preparation of endophyte treatments: Spore solutions are made by rinsing and scraping spores from agar slants which have been growing for about 1 month. Rinsing is done with 0.05% Silwet. Solutions are passed through Miracloth to filter out mycelia. Spores per ml are counted under a microscope using a hemocytometer. The stock suspension is then diluted into 10\hat{ }6 spores/ml utilizing water. 3 μl of spore suspension is used per soy seed (~10\hat{ }3 CFUs/seed is obtained). Control treatments are prepared by adding equivalent volumes of sterile water to seeds.

Assay of seedling vigor: Two rolled pieces of germination paper are placed in a sterile glass gar with 50 ml sterile water, then removed when completely saturated. Then the papers are separated and inoculated seeds are placed at approximately 1 cm intervals along the length of one sheet of moistened germination paper, at least 2.5 cm from the top of the paper and 3.8 cm from the edge of the paper. The second sheet of germination paper is placed on top of the soy seeds and the layered papers and seeds are loosely rolled into a tube. Each tube is secured with a rubber band around the middle, placed in a single sterile glass jar, and covered loosely with a lid. For each treatment, three jars with 15 seeds per jar are prepared. The position of jars within the growth chamber is randomized. Jars are incubated at 60% relative humidity, and 22° C. day, 18° C. night with 12 hours light and 12 hours dark for 4 days and then the lids are removed, and the jars incubated for an additional 7 days. Then the germinated soy seedlings are weighed and photographed, and root length and root surface area are measured.

Dirt, excess water, seed coats and other debris are removed from seedlings to allow accurate scanning of the roots. Individual seedlings are laid out on clear plastic trays and trays are arranged on an Epson Expression 11000XL scanner (Epson America, Inc., Long Beach CA). Roots are manually arranged to reduce the amount of overlap. For root measurements, shoots are removed if the shape of the shoot causes it to overlap the roots.

The WinRHIZO software version Arabidopsis Pro2016a (Regents Instruments, Quebec Canada) is used with the following acquisition settings: greyscale 4000 dpi image, speed priority, overlapping (1 object), Root Morphology: Precision (standard), Crossing Detection (normal). The scanning area is set to the maximum scanner area. When the scan is completed, the root area is selected, and root length and root surface area are measured.

Statistical analysis is performed using R (R Core Team, 2016. R: A language and environment for statistical computing. R Foundation for Statistical Computing, Vienna, Austria. R-project.org/) or a similar statistical software program.

### Assay of Corn Seedling Vigor

Seed preparation: The lot quality of corn seeds is first evaluated for germination by transfer of 100 seeds with 3.5 ml of water to a filter paper lined petri dish. Seeds are incubated for 3 days at 24° C. The process should be repeated with a fresh seed lot if fewer than 95% of the seeds have germinated. One thousand corn seeds are then surface sterilized by co-incubation with chlorine gas in a 20×30 cm container in a chemical fume hood for 12 hours. Percent germination of 50 seeds, per sterilization batch, is tested as above and confirmed to be greater than 95%.

Optional reagent preparation: 7.5% PEG 6000 (Calbiochem, San Diego, CA) is prepared by adding 75 g of PEG to 1000 ml of water, then stirred on a warm hot plate until the PEG is fully dissolved. The solution is then autoclaved.

Preparation of endophyte treatments: Spore solutions are made by rinsing and scraping spores from agar slants which have been growing for about 1 month. Rinsing is done with 0.05% Silwet. Solutions are passed through Miracloth to filter out mycelia. Spores per ml are counted under a microscope using a hemocytometer. The stock suspension is then diluted into 10\hat{ }6 spores/ml utilizing water. 3 μl of spore suspension is used per corn seed (~10\hat{ }3 CFUs/seed is obtained). Control treatments are prepared by adding equivalent volumes of sterile water to seeds.

Assay of seedling vigor: Either 25 ml of sterile water or, optionally, 25 ml of PEG solution as prepared above, is added to each CygTM germination pouch (Mega International, Newport, MN) and placed into pouch rack (Mega International, Newport, MN). Sterile forceps are used to place corn seeds prepared as above into every other perforation in the germination pouch. Seeds are fitted snugly into each perforation to ensure they do not shift when moving the pouches. Before and in between treatments forceps are sterilized using ethanol and flame and workspace wiped down with 70% ethanol. For each treatment, three pouches with 15 seeds per pouch are prepared. The germination racks with germination pouches are placed into plastic tubs and covered with perforated plastic wrap to prevent drying. Tubs are incubated at 60% relative humidity, and 22° C. day, 18° C. night with 12 hours light and 12 hours dark for 6 days to allow for germination and root length growth. Placement of pouches within racks and racks/tubs within the growth chamber is randomized to minimize positional effect. At the end of 6 days the corn seeds are scored manually for germination, root and shoot length.

Statistical analysis is performed using R or a similar statistical software program.

### Assay of Wheat Seedling Vigor

Seed preparation: The lot of wheat seeds is first evaluated for germination by transfer of 100 seeds and with 8 ml of water to a filter paper lined petri dish. Seeds are incubated for 3 days at 24° C. The process should be repeated with a fresh seed lot if fewer than 95% of the seeds have germinated. Wheat seeds are then surface sterilized by co-incubation with chlorine gas in a 20×30 cm container in a chemical fume hood for 12 hours. Percent germination of 50 seeds, per sterilization batch, is tested as above and confirmed to be greater than 95%.

Optional reagent preparation: 7.5% polyethylene glycol (PEG) is prepared by adding 75 g of PEG to 1000 ml of water, then stirring on a warm hot plate until the PEG is fully dissolved. The solution is then autoclaved.

Preparation of endophyte treatments: Spore solutions are made by rinsing and scraping spores from agar slants which have been growing for about 1 month. Rinsing is done with 0.05% Silwet. Solutions are passed through Miracloth to filter out mycelia. Spores per ml are counted under a microscope using a hemocytometer. The stock suspension is then diluted into 10\hat{ }6 spores/ml utilizing water. 3 μl of spore suspension is used per wheat seed (~10\hat{ }3 CFUs/seed was obtained). Seeds and spores are combined in a 50 ml falcon tube and gently shaken for 5-10 seconds until thoroughly coated. Control treatments are prepared by adding equivalent volumes of sterile water to seeds.

Assay of seedling vigor: Petri dishes are prepared by adding four sheets of sterile heavy weight seed germination paper, then adding either 50 ml of sterile water or, optionally, 50 ml of PEG solution as prepared above, to each plate then allowing the liquid to thoroughly soak into all sheets. The sheets are positioned and then creased so that the back of the plate and one side wall are covered, two sheets are then removed and placed on a sterile surface. Along the edge of the plate across from the covered side wall 15 inoculated wheat seeds are placed evenly at least one inch from the top of the plate and half an inch from the sides. Seeds are placed smooth side up and with the pointed end of the seed pointing toward the side wall of the plate covered by germination paper. The seeds are then covered by the two reserved sheets, and the moist paper layers smoothed together to remove air bubbles and secure the seeds, and then the lid is replaced. For each treatment, at least three plates with 15 seeds per plate are prepared. The plates are then randomly distributed into stacks of 8-12 plates and a plate without seeds is placed on the top. The stacks are incubated at 60% relative humidity, and 22° C. day, 18° C. night with 12 hours light and 12 hours dark for 24 hours, then each plate is turned to a semi-vertical position with the side wall covered by paper at the bottom. The plates are incubated for an additional 5 days, then wheat seeds are scored manually for scored manually for germination, root and shoot length, root and shoot surface area, seedling mass, and seedling length.

Statistical analysis is performed using R or a similar statistical software program.

### Assay of Rice Seedling Vigor

Seed preparation: The lot of rice seeds is first evaluated for germination by transfer of 100 seeds and with 8 ml of water to a filter paper lined petri dish. Seeds are incubated for 3 days at 24° C. The process should be repeated with a fresh seed lot if fewer than 95% of the seeds have germinated. Rice seeds are then surface sterilized by co-incubation with chlorine gas in a 20×30 cm container in a chemical fume hood for 12 hours. Percent germination of 50 seeds, per sterilization batch, is tested as above and confirmed to be greater than 95%.

Optional reagent preparation: 7.5% polyethylene glycol (PEG) is prepared by adding 75 g of PEG to 1000 ml of water, then stirring on a warm hot plate until the PEG is fully dissolved. The solution is then autoclaved.

Preparation of endophyte treatments: Spore solutions are made by rinsing and scraping spores from agar slants which have been growing for about 1 month. Rinsing was done with 0.05% Silwet. Solutions are passed through Miracloth to filter out mycelia. Spores per ml are counted under a microscope using a hemocytometer. The stock suspension is then diluted into 10\hat{ }6 spores/ml utilizing water. 3 μl of spore suspension is used per rice seed (~10\hat{ }3 CFUs/seed was obtained). Seeds and spores are combined in a 50 ml falcon tube and gently shaken for 5-10 seconds until thoroughly coated. Control treatments are prepared by adding equivalent volumes of sterile water to seeds.

Assay of seedling vigor: Petri dishes are prepared by adding four sheets of sterile heavy weight seed germination paper, then adding either 50 ml of sterile water or, optionally, 50 ml of PEG solution as prepared above, to each plate then allowing the liquid to thoroughly soak into all sheets. The sheets are positioned and then creased so that the back of the plate and one side wall are covered, two sheets are then removed and placed on a sterile surface. Along the edge of the plate across from the covered side wall 15 inoculated rice seeds are placed evenly at least one inch from the top of the plate and half an inch from the sides. Seeds are placed smooth side up and with the pointed end of the seed pointing toward the side wall of the plate covered by germination paper. The seeds are then covered by the two reserved sheets, and the moist paper layers smoothed together to remove air bubbles and secure the seeds, and then the lid is replaced. For each treatment, at least three plates with 15 seeds per plate are prepared. The plates are then randomly distributed into stacks of 8-12 plates and a plate without seeds is placed on the top. The stacks are incubated at 60% relative humidity, and 22° C. day, 18° C. night with 12 hours light and 12 hours dark for 24 hours, then each plate is turned to a semi-vertical position with the side wall covered by paper at the bottom. The plates are incubated for an additional 5 days, then rice seeds are scored manually for germination, root and shoot length.

Statistical analysis is performed using R or a similar statistical software program.

### Example 7. Greenhouse Assessment of Improved Plant Characteristics Under Water Deficit

This example describes an exemplary method by which improved plant health of endophyte treated plants may be shown in a growth environment comprising a water deficit. Greenhouse assay setup: This greenhouse assay is conducted in individual plastic containers filled with soil. The soil-filled conetainers for the stress condition are not moistened. The soil-filled conetainers for the non-stress condition are thoroughly moistened by top watering with approximately 5 L of water as well as absorbing water from the bottom of the conetainers (approximately 3 L) for at least 1 hour prior to planting. Stress treatment containers are watered with 1L of water immediately before planting. An additional conetainer is prepared for each conetainer to be planted. These conetainers are filled with 30 cc of pea gravel. The soil-filled conetainers are each placed into a gravel filled conetainer (also referred to as a secondary conetainer). This greenhouse assay was conducted using soybean seeds treated with a commercial Bradyrhizobiym seed treatment and Bradyrhizobiym treated seeds are either coated with an endophyte synthetic composition or left untreated as untreated controls (lacking formulation and the one or more heterologously disposed endophyte) as described herein. Seeds are placed into each pot and lightly covered with potting soil. Replicated conetainers of each treatment and stress condition are placed in conetainer racks in a Latin square design. The trays of conetainers are lightly covered and placed in a growth chamber. 48 hours after planting the covers are removed from the trays and all treatments are watered from the top with 1L of water. At 48 hours after planting the conetainer tray containing all treatments is watered from the bottom with 3.5L water, such that the water level just reaches the drain holes of the secondary conetainers; and the water level is maintained at this level throughout the experiment. Plants are harvested at 13-14 days post planting. The mass of the root tissue extending from the soil container is trimmed and weighted for each plant, and plant height is observed.

### Example 8. Greenhouse Assessment of Improved Plant Characteristics Under Nitrogen Deficit

This example describes an exemplary method by which improved plant health of endophyte treated plants may be shown in a growth environment comprising a nitrogen deficit.

Greenhouse assay setup: This greenhouse assay is conducted in individual plastic pots, filled with moistened potting soil. This greenhouse assay is conducted using seeds (optionally, chemically treated) coated with one or more endophytes described herein and formulation control (lacking the one or more heterologously disposed endophytes) and untreated controls (lacking formulation and the one or more heterologously disposed endophyte) as described in Example 3. Seeds are placed onto each pot and lightly covered with potting soil. Replicated pots of each treatment are set up and placed on a greenhouse bench using a random block design. For example, 18 replicates are planted for each treatment and control. Nitrogen deficit is introduced by reducing the Nitrogen in the Hoagland's solution (3 mM N), which is used to water the plants. Plants are monitored daily for emergence and watered as necessary to maintain a moist but not saturated soil surface (for example, plants are watered with 125 ml Hoagland's solution (3 mM N) per pot on every Monday, Wednesday, and Friday).

The following growth and vigor metrics are collected for each treatment: percentage emergence at Day 4, 5, 7 (for soybean, winter wheat and cotton) or Day 3, 4, 5 (for corn), leaf count (the number of fully expanded leaves on the main stem) at Days 10, 17 and 24.

Additional vigor and growth metrics may be collected including shoot height, leaf area, number of chlorotic leaves, chlorophyll content, number of live leaves, etc. At harvest, plants are gently removed from pots, washed with tap water to remove dirt, and photographed. Plant tissue is collected for nutrient composition analysis. Plants are put into a paper bag and dried in an oven. Optionally, the plant is separated into shoot and root tissue prior to drying. The dry weight of each individual plant, or shoot or root thereof, is recorded.

### Example 9. Greenhouse Assessment of Improved Plant Characteristics Under Phosphorus Deficit

This example describes an exemplary method by which improved plant health of endophyte treated plants may be shown in a growth environment comprising a phosphorus deficit.

This greenhouse assay is conducted in individual plastic pots, filled with moistened potting soil. This greenhouse assay is conducted using seeds (optionally, chemically treated) coated with one or more endophytes described herein and formulation control (lacking the one or more heterologously disposed endophytes) and untreated controls (lacking formulation and the one or more heterologously disposed endophyte) as described in Example 4. Seeds are placed onto each pot and lightly covered with potting soil. Replicated pots of each treatment are set up and placed on a greenhouse bench using a random block design. For example, 16 replicates are planted for each treatment and control. Phosphorus deficit is introduced by removing Phosphorus from the Hoagland's solution (0 mM P), which is used to water the plants. Plants are monitored daily for emergence and watered as necessary to maintain a moist but not saturated soil surface (for example, plants are watered with 125 ml Hoagland's solution (0 mM P) per pot on every Monday, Wednesday, and Friday).

The following growth and vigor metrics are collected for each treatment: percentage emergence at Day 4, 5, 7 (for soybean, winter wheat and cotton) or Day 3, 4, 5 (for corn), leaf count (the number of fully expanded leaves on the main stem) at Days 10, 17 and 24.

Additional vigor and growth metrics may be collected including shoot height, leaf area, coloration of leaves, number of live leaves, etc. At harvest, plants are gently removed from pots, washed with tap water to remove dirt, and photographed. Plant tissue is collected for nutrient composition analysis. Plants are put into a paper bag and dried in an oven. Optionally, the plant is separated into shoot and root tissue prior to drying. The dry weight of each individual plant, or shoot or root thereof, is recorded.

### Example 10. Greenhouse Assessment of Improved Plant Health Under Biotic Stress

This example describes an exemplary method by which improved plant health of endophyte treated plants was shown in a growth environment comprising the crop pathogen Pythium ultimum or Rhizoctonia solani. This assay utilized cotton, soybeans, winter wheat, or corn.

Preparation of pathogen inoculum A stock of Rhizoctonia solani anastomosis group 4 or Pythium ultimum var. ultimum is grown on a standard potato dextrose agar plate. Plugs of fresh mycelium were then transferred into standard potato dextrose broth. After sufficient growth was achieved, the culture was poured though cheesecloth to capture the fungal biomass, which was subsequently rinsed with water. After removing excess rinsate, a roughly equivalent volume of water was added to the fungal biomass before blending to create a slurry. The resulting slurry was further diluted to the required concentration necessary to observe desired level of symptoms.

Greenhouse assay setup The greenhouse assay was conducted in a commercial potting soil. A divot was placed in the center of a pot containing wetted soil using a standardized dibble. An appropriate volume of slurry was added to the center of each divot. An equivalent volume of water was added for control treatments.

This greenhouse assay was conducted using seeds coated with one or more endophytes described herein in PBS and untreated controls (lacking endophyte). Seeds were placed onto each divot after addition of the inoculum. The seeds were then covered with uninoculated soil and again watered. High soil moisture levels were maintained throughout the course of the experiment. Fourteen replicates were included in a randomized design to obtain sufficient statistical power for analysis, this assay was repeated. Plants were grown in a controlled environment until 7 days post emergence of control plants. Shoot fresh weight was measured on a per plant basis.

TABLE 5
Plant phenotypes under biotic stress of endophyte-treated and control-treated plants

| | | | Value | Treatment | | |
|---|---|---|---|---|---|---|
| Treatment | Metric | Value | Descriptor | Concentration | Crop | Condition |
| MIC-54924 | Shoot Fresh | 58.01 | percent change | 1E6 | Cotton | Pythium |
| | Weight | | untreated control | cells/mL | | ultimum |
| MIC-54924 | Shoot Fresh | −52.71 | percent change | 0.05 OD | Cotton | Pythium |
| | Weight | | untreated control | | | ultimum |
| MIC-54924 | Shoot Fresh | 14.85 | percent change | 0.05 OD | Cotton | Pythium |
| | Weight | | untreated control | | | ultimum |
| MIC-54924 | Shoot Fresh | 36.38 | percent change | 3.33E7 | Soy | Pythium |
| | Weight | | untreated control | cells/mL | | ultimum |
| MIC-54924 | Shoot Fresh | 34.39 | percent change | 1E6 | Soy | Pythium |
| | Weight | | untreated control | cells/mL | | ultimum |
| MIC-54924 | Shoot Fresh | 54.01 | percent change | 3.33E7 | Soy | Pythium |
| | Weight | | untreated control | CFU/mL | | ultimum |
| MIC-54924 | Shoot Fresh | 2.65 | percent change | 3.33E7 | Soy | Pythium |
| | Weight | | untreated control | cells/mL | | ultimum |
| MIC-54924 | Shoot Fresh | −30.02 | percent change | 3.33E7 | Soy | Pythium |
| | Weight | | untreated control | cells/mL | | ultimum |
| MIC-54924 | Shoot Fresh | −34.08 | percent change | 3.33E7 | Soy | Pythium |
| | Weight | | untreated control | cells/mL | | ultimum |
| MIC-54924 | Shoot Fresh | 2.05 | percent change | 3.33E7 | Soy | Pythium |
| | Weight | | untreated control | cells/mL | | ultimum |
| MIC-54924 | Shoot Fresh | 34.84 | percent change | 10\hat{ }6 | Soy | Pythium |
| | Weight | | untreated control | | | ultimum |
| MIC-54924 | Shoot Fresh | −11.39 | percent change | 3.33E7 | Soy | Pythium |
| | Weight | | untreated control | CFU/mL | | ultimum |
| MIC-54924 | Shoot Fresh | 64.45 | percent change | 1E6 | Winter | Pythium |
| | Weight | | untreated control | cells/mL | Wheat | ultimum |
| MIC-54924 | Shoot Fresh | 225.18 | percent change | 3.33E7 | Winter | Pythium |
| | Weight | | untreated control | CFU/mL | Wheat | ultimum |
| MIC-54924 | Shoot Fresh | 17.79 | percent change | 3.33E7 | Winter | Pythium |
| | Weight | | untreated control | cells/mL | Wheat | ultimum |
| MIC-54924 | Shoot Fresh | 59.96 | percent change | 3.33E7 | Winter | Pythium |
| | Weight | | untreated control | cells/mL | Wheat | ultimum |
| MIC-54924 | Shoot Fresh | 54.9 | percent change | 3.33E7 | Winter | Pythium |
| | Weight | | untreated control | CFU/mL | Wheat | ultimum |
| MIC-54924 | Shoot Fresh | −3.3 | percent change | 3.33E7 | Corn | Rhizoctonia |
| | Weight | | untreated control | cells/mL | | solani |
| MIC-54924 | Shoot Fresh | 12.18 | percent change | 6.66E7 | Corn | Rhizoctonia |
| | Weight | | untreated control | cells/mL | | solani |
| MIC-54924 | Shoot Fresh | −24.49 | percent change | 3.33E7 | Corn | Rhizoctonia |
| | Weight | | untreated control | cells/mL | | solani |
| MIC-54924 | Shoot Fresh | −52.27 | percent change | 0.05 OD | Cotton | Rhizoctonia |
| | Weight | | untreated control | | | solani |
| MIC-54924 | Shoot Fresh | −13.59 | percent change | 3.33E7 | Soy | Rhizoctonia |
| | Weight | | untreated control | cells/mL | | solani |
| MIC-54924 | Shoot Fresh | 53.46 | percent change | 3.33E7 | Soy | Rhizoctonia |
| | Weight | | untreated control | cells/mL | | solani |
| MIC-54924 | Shoot Fresh | 8.24 | percent change | 3.33E7 | Soy | Rhizoctonia |
| | Weight | | untreated control | CFU/mL | | solani |
| MIC-54924 | Shoot Fresh | 1.73 | percent change | 10\hat{ }6 | Soy | Rhizoctonia |
| | Weight | | untreated control | | | solani |
| MIC-54924 | Shoot Fresh | 37.84 | percent change | 3.33E7 | Soy | Rhizoctonia |
| | Weight | | untreated control | cells/mL | | solani |
| MIC-54924 | Shoot Fresh | −26.04 | percent change | 3.33E7 | Soy | Rhizoctonia |
| | Weight | | untreated control | CFU/mL | | solani |
| MIC-54924 | Shoot Fresh | −57.56 | percent change | 3.33E7 | Winter | Rhizoctonia |
| | Weight | | untreated control | cells/mL | Wheat | solani |
| MIC-54924 | Shoot Fresh | −40.11 | percent change | 10\hat{ }6 | Winter | Rhizoctonia |
| | Weight | | untreated control | | Wheat | solani |
| MIC-54924 | Shoot Fresh | 91.55 | percent change | 3.33E7 | Winter | Rhizoctonia |
| | Weight | | untreated control | cells/mL | Wheat | solani |

TABLE 6
Summary of plant phenotypes under biotic stress growth chamber testing

| | | | | | Average percent | |
|---|---|---|---|---|---|---|
| | | | | Number | change from | Win |
| Treatment | Crop | Pathogen | Metric | of runs | untreated control | Rate |
| MIC-54924 | Corn | Pythium | Shoot Fresh | 2 | 4.11 | 50% |
| | | aphanidermatum | Weight | | | |
| MIC-54924 | Corn | Rhizoctonia | Shoot Fresh | 5 | −5.58 | 40% |
| | | solani | Weight | | | |
| MIC-54924 | Cotton | Pythium | Shoot Fresh | 3 | 6.43 | 67% |
| | | ultimum | Weight | | | |
| MIC-54924 | Cotton | Rhizoctonia | Shoot Fresh | 1 | −51.55 | 0% |
| | | solani | Weight | | | |
| MIC-54924 | Soybean | Pythium | Shoot Fresh | 9 | 10.49 | 67% |
| | | ultimum | Weight | | | |
| MIC-54924 | Soybean | Rhizoctonia | Shoot Fresh | 7 | 13.62 | 71% |
| | | solani | Weight | | | |
| MIC-54924 | Winter | Pythium | Shoot Fresh | 8 | 101.27 | 100% |
| | Wheat | ultimum | Weight | | | |
| MIC-54924 | Winter | Rhizoctonia | Shoot Fresh | 5 | 18.47 | 50% |
| | Wheat | solani | Weight | | | |

### Example 11. Greenhouse Assessment of Improved Plant Health Under Biotic Stress

This example describes an exemplary method by which improved plant health of endophyte treated plants were shown in a growth environment comprising the crop pathogen Fusarium sp., one of the causal agents of seedling damping off disease. This assay may utilize dicots or monocots, including, for example, soybean or wheat.

Preparation of Fusarium sp. inoculum A stock of Fusarium sp. was grown on a standard potato dextrose agar plate. Plugs of fresh mycelium were then transferred into breathable bag containing a sterile mixture of water and grain such as sorghum or millet. After sufficient growth was achieved, the culture was removed from the bags and dried. After drying the biomass was coarsely ground.

Greenhouse assay setup The greenhouse assay was conducted in a media mixture consisting of a commercial potting soil and a minimum of 50% inert inorganic material such as calcined clay or vermiculite or pearlite. An appropriate volume of ground pathogen was added to the soil mixture to obtain desired level of symptoms.

This greenhouse assay was conducted using seeds coated with one or more endophytes described herein in PBS formulation and untreated controls (lacking endophyte). A seed was added to the surface of the infested media. The seed was then covered with media lacking pathogen and again watered. High soil moisture levels were maintained throughout the course of the experiment. Fourteen replicates were included in a randomized design to obtain sufficient statistical power for analysis; this assay was repeated. Shoot fresh weight was measured on a per plant basis.

TABLE 7
Plant phenotypes under biotic stress (Fusarium oxysporum)
of endophyte-treated and control-treated plants

| | | | | Treatment | | Measurement |
|---|---|---|---|---|---|---|
| Treatment | Metric | Value | Value Descriptor | Concentration | Crop | Time |
| MIC-54924 | Shoot Fresh | 28.33 | percent change | 3.33E7 | Soybeans | 11 days after |
| | Weight | | untreated control | cells/mL | | planting |
| MIC-54924 | Shoot Fresh | −48.85 | percent change | 1E6 | Soybeans | 10 days after |
| | Weight | | untreated control | cells/mL | | planting |
| MIC-54924 | Shoot Fresh | −10.06 | percent change | 3.33E7 | Soybeans | 11 days after |
| | Weight | | untreated control | cells/mL | | planting |
| MIC-54924 | Shoot Fresh | 8.88 | percent change | 3.33E8 | Soybeans | 11 days after |
| | Weight | | untreated control | cells/mL | | planting |
| MIC-54924 | Shoot Fresh | 4.91 | percent change | 3.33E7 | Soybeans | 11 days after |
| | Weight | | untreated control | cells/mL | | planting |
| MIC-54924 | Shoot Fresh | 45.51 | percent change | 3.33E6 | Soybeans | 11 days after |
| | Weight | | untreated control | cells/mL | | planting |
| MIC-54924 | Shoot Fresh | −10.33 | percent change | 1E6 | Winter | 7 days after |
| | Weight | | untreated control | cells/mL | Wheat | planting |
| MIC-54924 | Shoot Fresh | 27.32 | percent change | 3.33E7 | Winter | 8 days after |
| | Weight | | untreated control | cells/mL | Wheat | planting |
| MIC-54924 | Shoot Fresh | 5.11 | percent change | 3.33E7 | Winter | 8 days after |
| | Weight | | untreated control | cells/mL | Wheat | planting |
| MIC-54924 | Shoot Fresh | 35.29 | percent change | 3.33E7 | Winter | 8 days after |
| | Weight | | untreated control | cells/mL | Wheat | planting |
| MIC-54924 | Shoot Fresh | 55.39 | percent change | 3.33E7 | Winter | 8 days after |
| | Weight | | untreated control | cells/mL | Wheat | planting |
| MIC-54924 | Shoot Fresh | 17.7 | percent change | 3.33E6 | Winter | 9 days after |
| | Weight | | untreated control | cells/mL | Wheat | planting |

TABLE 8
Summary of results of Fusarium growth chamber testing

| | | | | | Average percent | |
|---|---|---|---|---|---|---|
| | | | | Number of | change from | Win |
| Treatment | Crop | Pathogen | Metric | runs | untreated control | Rate |
| MIC- | Corn | Fusarium | Shoot Fresh | 4 | 11.68 | 75% |
| 54924 | | graminearum | Weight | | | |
| MIC- | Soybean | Fusarium | Shoot Fresh | 6 | 4.49 | 67% |
| 54924 | | oxysporum | Weight | | | |
| MIC- | Soybean | Fusarium | Shoot Fresh | 6 | 5.20 | 67% |
| 54924 | | virguliforme | Weight | | | |
| MIC- | Winter | Fusarium | Shoot Fresh | 3 | 5.08 | 33% |
| 54924 | Wheat | graminearum | Weight | | | |
| MIC- | Winter | Fusarium | Shoot Fresh | 16 | 15.68 | 81% |
| 54924 | Wheat | oxysporum | Weight | | | |

### Example 12. In Vitro Assessment of Production of Antibiotic Metabolites Using Live Endophyte Cultures

This example describes an exemplary method by which microbes may be shown to inhibit the growth of hyphal phytopathogens in vitro. Such phytopathogens can be members of the “true” fungi, phylum Eumycota, or from other taxonomic groups with a similar growth habit such as members of the phylum Oomycota. Hyphal growth can be described as organism growth along thread-like structures composed of connected cells. Such growth is found commonly among fungi and oomycetes, and even some genera of bacteria. In this assay, the hyphal growth should be in a roughly uniform, radial manner. This assay is comprised of a Petri plate containing an agar-based media and a hyphal phytopathogen grown concomitantly a live endophyte. This assay is completed using pathogens: Cercospora sojina, Colletotrichum truncatum, Corynespora cassicola, Diaporthe aspalathi, Fusarium graminearum, Macrophomina phaseolina, Pythium debaryanum, Rhizoctonia solani, and Sclerotinia sclerotiorum.

Preparation of Hyphal Phytopathogen A Petri plate containing a media suitable for the growth of the target hyphal pathogen was inoculated with the target hyphal pathogen. The initial inoculum should be from an axenic culture, but non-axenic cultures containing stable endophytes may also be used. Any media may be used that supports healthy growth of the hyphal pathogen. After inoculation on the media-containing Petri plate, the culture was allowed to grow until reaching the edge of the Petri plate. A test pathogen sample was collected from this plate.

Preparation of the test sample The microbial samples for testing, also referred to as test samples, were MIC-54924 in water dispersed formulation at three dosages: 0.5 mL/kg seeds, 1.3 mL/kg seeds, and 2 mL/kg seeds. Seeds treated with a commercial chemical fungicide (“Chemical Control”), and untreated controls were also prepared. Viable soybean seeds were used in these assays.

Alternatively, a liquid culture of either type of microbe can be grown, and viable material is removed by various methods including, but not limited to, filtration or autoclaving. This later method of testing a non-viable test sample is best used when the test microbe displays a much faster rate of radial growth than the hyphal pathogen being tested. This later method is also more sensitive at differentiating between the passive production of antimicrobial metabolites versus an active biological process such a mycophagy. Test samples from all these methods can also be applied to viable or devitalized seeds. Seed application replicates conditions a microbe might experience when used as a seed treatment, having physical and biochemical interactions with seed material.

Assay Set-Up A Petri dish containing a solid agar test media (herein referred to as the test plate) was obtained. A sterile instrument was used to remove a test pathogen plug from the hyphal pathogen plate culture described in Preparation of Hyphal Phytopathogen. This test pathogen plug was placed on a fresh solid agar plate. Next a test sample was applied to the test plate at a distance such that the test sample and test plate came into physical contact after more than one day of growth. Four treated seeds were placed on each plate approximately equidistant from the pathogen plug. Alternately, live microbe in formulation or fermentation media was applied directly to the test plate, or for assaying a non-viable test sample, an agar plug was removed from the test plate using a sterile instrument to create a well to hold the test sample. The well was then filled with the non-viable test sample, and the sample was absorbed into the agar media.

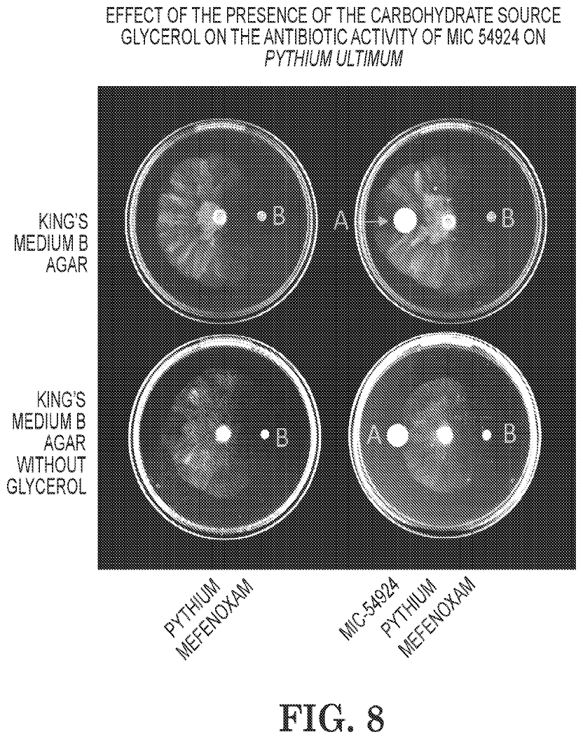

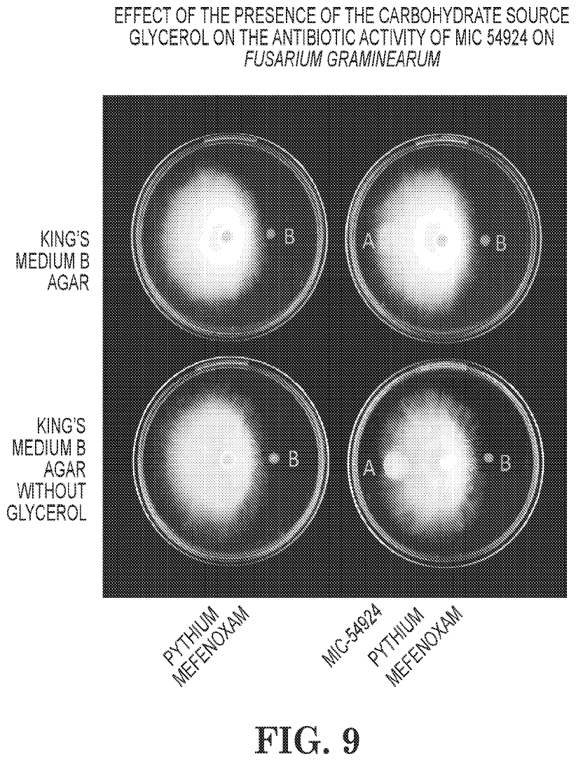

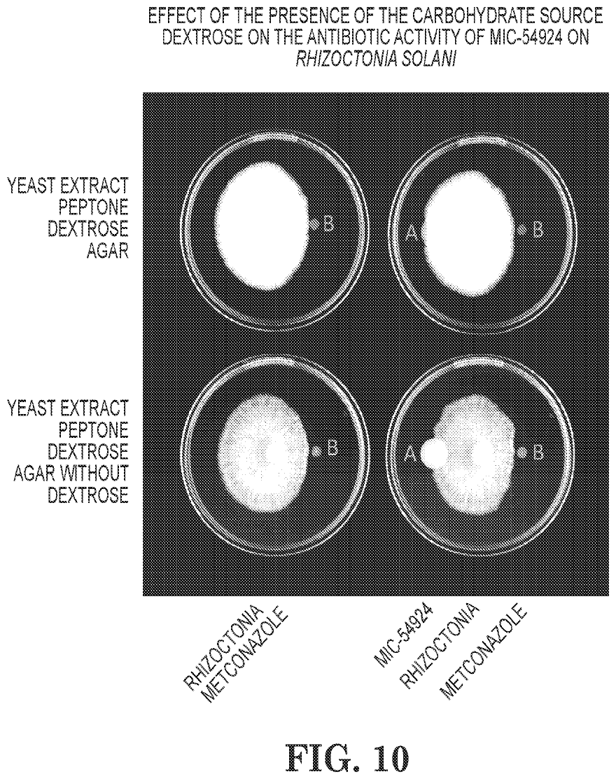

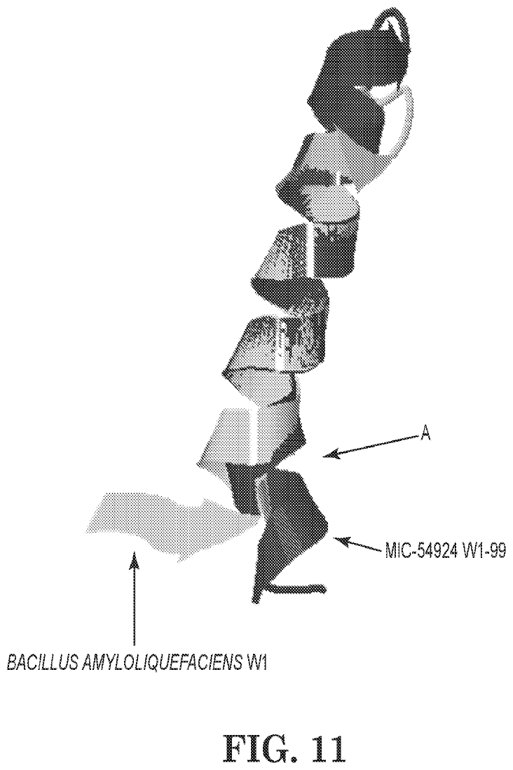

Use of Multiple Growth Media. This plate-based assay was repeated with multiple media types. Medias were chosen to vary important growth inputs such as carbon source, presence and concentration of various salts, and presence of extracts from different plant species or organs. FIGS. 8, 9, and 10 show examples of use of differing media (King's medium B agar and yeast extract peptone dextrose agar), where the assays were also run without selected carbon sources (glycerol in FIGS. 8 and 9, dextrose in FIG. 11). MIC-54924 showed a greater zone of inhibition of fungal pathogens when in nutrient scarce conditions. MIC-54924 showed enhanced inhibition of Pythium ultimum and Fusarium graminearum in the absence of glycerol. MIC-54924 showed enhanced inhibition of Rhizoctonia solani in the absence of glycerol.

Assessment After setting up, hyphal pathogens were allowed to grow for sufficient time such that the hyphal front met or just passed the test sample and analyzed at 2, 4, 6, 8, 10, and 12 days post plating. Plates were scored based on the degree of restriction of growth of the hyphal front around the test sample, clearing around the test sample, and comparison of the morphology of the hyphal pathogen near the test sample to areas away from the test sample. When anti-pathogen metabolites were not produced and secreted, the hyphal pathogen grew over the test sample with little to no visible effect on growth.

TABLE 9
Summary of observations of plate-based anti-biosis assays with MIC-54924 treated seeds

| | Observations of MIC-54924 treated seeds |
|---|---|
| Cercospora | Significant reduction in pathogen colony diameter relative to untreated controls at all dosages |
| sojina | at 4, 6, 8, 10, and 12 days post plating. Colony diameters of pathogen colonies on plates |
| | containing seeds treated with MIC-54924 were smaller than those on plates containing |
| | Chemical Control treated seeds at days 6, 8, 10, and 12 post plating. |
| Colletotrichum | Significant reduction in pathogen colony diameter relative to untreated controls at all dosages |
| truncatum | at 2, 4, 8, and 10 days post plating. |
| Corynespora | Significant reduction in pathogen colony diameter relative to untreated controls at all dosages |
| cassicola | at 4, 6, 8, 10, and 12 days post plating. Colony diameters of pathogen colonies on plates |
| | containing seeds treated with MIC-54924 were smaller than those on plates containing |
| | Chemical Control treated seeds at days 6, 8, 10, and 12 post plating. |
| Diaporthe | Significant reduction in pathogen colony diameter relative to untreated controls at all dosages |
| aspalathi | at 2, 4, and 6 days post plating. |
| Fusarium | Significant reduction in pathogen colony diameter relative to untreated controls at all dosages |
| graminearum | at 4 days post plating. |
| Macrophomina | Significant reduction in pathogen colony diameter relative to untreated controls at all dosages |
| phaseolina | at 4 days post plating. |
| Pythium | No significant reduction in pathogen colony diameters on any days. |
| debaryanum | |
| Rhizoctonia | Significant reduction in pathogen colony diameter relative to untreated controls at all dosages |
| solani | at 2. Significant reduction at day 4 for the 0.5 mL/kg and 1.3 mL/kg dosages, but not the 2 |
| | mL/kg dosage. Significant reduction at day 6 for the 2 mL/kg dosage, but not the 0.5 mL/kg |
| | and 1.3 mL/kg dosages. |
| Sclerotinia | No significant reduction in pathogen colony diameters on any days. |
| sclerotiorum | |

### Example 13. In Vitro Assessment of Production of Antibiotic Metabolites Using Filtered or Dead Endophyte Cultures

This example describes an exemplary method by which microbes may be shown to produce metabolites that inhibit the growth of hyphal phytopathogens in vitro. Such phytopathogens can be members of the “true” fungi, phylum Eumycota, or from other taxonomic groups with a similar growth habit such as members of the phylum Oomycota. Hyphal growth can be described as organism growth along thread-like structures composed of connected cells. Such growth is found commonly among fungi and oomycetes, and even some genera of bacteria. In this assay, the hyphal growth should be in a roughly uniform, radial manner. This assay is comprised of a Petri plate containing an agar-based media and a hyphal phytopathogen grown in the presence of the spent media from a previously grown endophyte.

Preparation of Hyphal Phytopathogen A Petri plate containing a media suitable for the growth of the target hyphal pathogen is inoculated with the target hyphal pathogen. The initial inoculum should be from an axenic culture, but non-axenic cultures containing stable endophytes may also be used. Any media can be used that supports healthy growth of the hyphal pathogen. After inoculation on the media-containing Petri plate, the culture is allowed to grow until reaching the edge of the Petri plate. A test pathogen sample will be collected from this plate as a hyphal plug. Alternatively, hyphal phytopathogens can also be grown in a liquid media known to induce the production of spores. Spores are then collected either from this liquid media or from liquid applied to the plate described above. The spores are then collected via filtration and/or centrifugation.

Preparation of the test sample A microbial sample for testing, also referred to as a test sample, can be produced in multiple ways. A liquid culture of hyphal or colony forming microbe is grown in liquid culture, and viable material is removed by various methods including filtration and/or centrifugation. Alternately, or in addition to filtration, a test sample may be devitalized, and the non-viable test sample may be used. This later method of testing a non-viable test sample is used when the test microbe displays a much faster rate of radial growth than the hyphal pathogen being tested, to identify production of antimicrobial metabolites, for example not as a part an active biological process such a mycophagy.

Assay Set-Up A Petri dish containing a solid agar test media is obtained. This will be referred to as the test plate. The test sample can be applied to this solid agar test plate in multiple ways. The test sample can be added directly to the molten media prior to the plates being poured. The test sample can be applied to the surface of an already prepared solid agar test plate, spread evenly over the surface with excess liquid allowed to dry. Finally, an agar plug can be removed from the test plate using a sterile instrument to create a well to hold the test sample. The well is then filled with a test sample, and the sample is absorbed into the agar media. In addition to the test sample, a sterile instrument is used to remove a test pathogen plug from the hyphal pathogen plate culture and placed on the test plate. Alternatively, a spore or hyphal slurry from the phytopathogen can be applied to the test plate, either as a drop or spread evenly over the top of the plate with excess liquid allowed to dry.

Use of Multiple Growth Media Pathogens grown under various environmental conditions are expected to show differential sensitivity to those metabolites. For this reason, this assay is performed on multiple media types. Medias are chosen to vary important growth inputs such as carbon source, presence and concentration of various salts, and presence of extracts from different plant species or organs.

Assessment If applied as a hyphal plug or drop of slurry, hyphal pathogens are allowed to grow for sufficient time such that the hyphal front meets or just passes the test sample. In cases where anti-pathogen metabolites are produced and secreted, a restriction of growth of the hyphal front around the test sample is commonly observed. Often this will also result in an area of clearing around the test sample. In these cases, the morphology of the hyphal pathogen near the test sample will often also be dissimilar from areas away from the test sample. Alternatively, when anti-pathogen metabolites are not produced and secreted, the hyphal pathogen will grow over the test sample with little to no visible effect on growth.

If phytopathogen was applied across the surface of the plate, reduced growth can be observed microscopically shortly after application by looking at the germination of spores. An area of decreased pathogen growth around the test sample may also be observed in some embodiments of this assay. A relative decrease in transparency compared to a control plate may also be observed when the test sample is applied to the molten media or uniformly across the surface of the test plate.

### Example 14. Nematode Egg Inoculum Preparation

This example describes an exemplary method for obtaining nematode eggs for use in stock population maintenance, in planta screening assays, and for hatching for in vitro assays. The nematode species utilized are Meloidogyne incognita (Southern root-knot nematode, “RKN”), Heterodera glycines (Soybean cyst nematode, “SCN”), and Rotylenchulus reniformis (Reniform nematode, “REN”). Populations of nematodes may be obtained, for example from a stock crop of corn for RKN, cotton for REN, and soybean for SCN.

Experimental Preparation Eggs are extracted from nematode stock crops; RKN and REN are collected from plants that are ~60-75 days old, and SCN is collected from plants that are ~70-85 days old. The above ground biomass is removed and discarded. If multiples are being extracted, necessary precautions are taken to prevent cross contamination of nematode species.

RKN and REN Egg Extraction from Roots Soil is washed from the roots of infected stock crops and the roots are placed in a prepared container. To extract the nematodes, a 0.625% NaOCl solution is added to the container and the roots are agitated for 4 minutes using an orbital shaker set at approximately 100-120 rpm.

The NaOCl extraction solution is then poured through an 8″ diameter 25 μm pore sieve with an 8″ diameter 75 μm pore sieve stacked on top to sift out debris. The roots are manually scrubbed over the sieve stack while running water over them. Alternately the roots are placed in a blender with water and pulsed until macerated. If using a blender, the contents are poured back through the sieve stack. The 75 μm pore sieve is rinsed into the 25 μm pore sieve. Eggs are captured on the 25 μm pore sieve. The 25 μm pore sieve is held at an angle and gently rinsed with water to collect all the eggs into a small pool at the bottom. The eggs are carefully collected into a storage container using a wash bottle.

SCN Cyst Extraction from Soil. Soil is washed from the roots of infected stock crops and the excess soil and rinse water are collected in a small bucket. The roots are manually scrubbed to remove cysts that remain visibly stuck to the roots. Eight-inch sieves are stacked on top of a separate small bucket. An 850 μm pore sieve is on top and a 250 μm pore sieve is underneath. The collected soil and rinse water are mixed and then allowed to settle for 3 seconds before the liquid portion of the soil mixture is poured through the sieve stack. Water is added to the retained soil, and the mixing, settling, and pouring steps are repeated. After the second wash the remaining soil is discarded.

The 850 μm pore sieve is rinsed into the 250 μm pore sieve. Cysts are captured on the 250 μm pore sieve. The 250 μm pore sieve is held at an angle and gently rinsed with water to collect all the eggs into a small pool at the bottom. The cysts are carefully collected into a storage container using a wash bottle, using a minimal amount of water.

SCN Egg Extraction from Cysts Collected cysts are placed into a mortar, and thoroughly ground using a pestle. An 8″ 75 μm pore sieve is stacked on top of an 8″ 25 μm pore sieve and the mortar contents are washed through the sieves. The eggs are collected from the 25 μm pore sieve by rinsing the 75 μm pore sieve into the 25 μm pore sieve. Eggs are captured on the 25 μm pore sieve. The 25 μm pore sieve is held at an angle and gently rinsed with water to collect all the eggs into a small pool at the bottom. The eggs are carefully collected into a storage container using a wash bottle. The cyst mixture remaining on the 75 μm pore sieve is collected again and the grinding, sieving, and rinsing steps are repeated until the cysts are extracted.

Egg Centrifugation Eggs are further separated from small debris by centrifugation with sucrose. A sucrose solution is made by adding 495 g of white cane sugar into a 1L bottle and filling up to the 1L measurement with DI water. The mixture is stored at 4° C. until ready to use. Approximately 25 ml of sucrose solution is added to each 50 ml conical tube. Then the egg inoculum is mixed to evenly distribute eggs and the inoculum poured into the prepared conical tubes until the total inoculum volume is distributed. The tubes are then centrifuged at 1040 rpm for 1 minute. Nematode eggs float at the top of the solution in the centrifuged tubes. A sieve stack is made using 3″ diameter sieves, with a 75 μm pore sieve on top of a 25 μm pore sieve. The top half of the tube contents is poured though the sieves and rinsed with water to wash away the sugar solution. The eggs are collected from the 25 μm pore sieve by rinsing the 75 μm pore sieve into the 25 μm pore sieve. Eggs are captured on the 25 μm pore sieve. The 25 μm pore sieve is held at an angle and gently rinsed with water to collect all the eggs into a small pool at the bottom. The eggs are carefully collected into a storage container using a wash bottle. The eggs are enumerated at 40× magnification using an inverted microscope. Eggs to be used for in planta screening are standardized to 2000 eggs/mL.

### Example 15. In-Vitro Nematode Supernatant Assay

RKN and SCN eggs are collected as described above. A hatching environment is prepared by lining a small sterile container with a clean, wood fiber based delicate task tissue and saturating the tissue with deionized water. The collected eggs are mixed with a sugar solution and centrifuged at 240 g for one minute. The supernatant containing the eggs is poured through a 25 μm pore sieve. Approximately 250,000 to 500,000 eggs are added to the prepared hatching environment, and the hatching environment is incubated at 30° C. and shaken at 25 rpm in the dark. Deionized water is added to the hatching environment to ensure the water level does not fall to below the tissue, and at least every 3 days to ensure proper oxygenation. After 6 days, hatched second stage juveniles (J2) are rinsed through a stack of 45 μm and 25 μm sieves that have been previously sprayed with 70% ethanol and rinsed with deionized water. Sterilized deionized water is used to collect J2 from the 45 μm pore sieve into a sterile 100 mL glass beaker, and J2 concentration standardized to 30±5 per 10 μL with sterile deionized water. A control treatment is prepared by adding 2 μL abamectin to 78 μL of sterile deionized water per replicate.

In-vitro Supernatant Screening Protocol Ten μL of the prepared J2 suspension is added to wells of a 96well half area plate. One abamectin control is added to each plate. Additionally, one negative control (media lacking endophyte) is prepared for each plate. Sterilized deionized water and then endophyte supernatant (Total volume: 80 μL) are aliquoted in each treatment well of the 96-well plate to desired supernatant percentage. In a fume hood, 80 μL of the prepared abamectin is added into active wells of Control Plate. 10 μL of propidium iodide (0.2 mM) is added to reach a final concentration of 20 μM. Total well volume should equal 100 μL. Plates are sealed with a breathable membrane and stored in the dark at room temperature for 48 hours.

Intensity of propidium iodide in each well is measured using the propidium iodide filter on a BioTek Cytation 5 Cell Imaging Multimode Reader (Agilent, Santa Clara, CA, USA). The intensity of propidium iodide (which binds to dead cells) is proportional to the mortality of the incubated nematodes.

### Example 16. Greenhouse Assessment of Improved Plant Health Under Biotic Stress (Soybean Cyst Nematode)

This example describes an exemplary method by which improved plant health of endophyte treated plants may be shown in a growth environment comprising the crop pest soybean cyst nematode (Heterodera glycines).

Greenhouse assays are conducted using soybean seeds (optionally, chemically treated soybean seeds) coated with one or more of the endophytes described herein and formulation control (lacking the one or more heterologously disposed endophytes) and untreated controls (lacking formulation and the one or more heterologously disposed endophyte). Microbe treated soybean seeds are planted, infected with nematodes, maintained, and phenotyped in grow rooms.

In one embodiment, the following method is used. 98 cones are placed in each conetainer to obtain the needed number of conetainers. Masks are placed over cones and cones are filled with soil. The conetainer is placed in a deep pan and water is added until the soil in the cones is saturated. Two soybean seeds are planted 2.5 cm deep in each conetainer.

One ml containing 2,000 H. glycines eggs is pipetted into each cone at planting or the desired number of days after planting. Seedlings are thinned to one per cone after emergence and watered as appropriate.

Phenotyping is performed as follows. The height of each plant is measured, e.g., by placing the ruler on the lip of a cell and measuring the plant's height to the nearest millimeter. The mass of each plant is measured, e.g., by cutting the plant at the soil surface, placing the shoot in the weighing container, allowing the weight to stabilize, and autorecording the mass via the scale's software. The number of H. glycines cysts may be counted after extraction from soybean roots as described herein. The water suspension containing 150 cm\hat{ }3 of soil is poured through nested 75-μm and 25-μm-pore sieves to extract vermiform stages (juveniles and males). Vermiform stages are collected on the 75-μm-pore sieve and centrifuged using, e.g., the sucrose centrifugation-flotation method.

### Example 17. Greenhouse Assessment of Improved Plant Health Under Biotic Stress (Soybean Aphid)

This example describes an exemplary method by which improved plant health of endophyte treated plants may be shown in a growth environment comprising the crop pest soybean aphid (Aphis glycines).

Greenhouse assays are conducted using soybean seeds (optionally, chemically treated soybean seeds) coated with one or more of the endophytes described herein and formulation control (lacking the one or more heterologously disposed endophytes) and untreated controls (lacking formulation and the one or more heterologously disposed endophyte) as described in herein. Microbe treated soybean seeds are planted, infected with soybean aphids (Aphis glycines), maintained in grow rooms, and phenotyped.

In one embodiment, the following method is used. 98 cones are placed in each conetainer to obtain the needed number of conetainer. Masks are placed over cones and cones are filled with potting medium or soil. The conetainer is placed in a deep pan and water is added until the soil in the cones is saturated. One soybean seed is planted in each conetainer. Each conetainer is placed in a growth tub and watered.

A community of soybean aphids is maintained on a stock of soybean plants. To prepare for infestation of the experimental plants, leaves are removed from infested soybean plants from the stock community. One or more leaves are examined under a stereoscope to make sure the aphids are alive and vigorous. Infested leaf cutlets are placed in square plates to keep leaves alive until the treatment plants are infested with aphids. In some embodiments, 20 infested leaf cutlets are used per each 98-cone tray used in the experiment. The infested leaf cutlets are introduced to the growth environment of the experimental plants at planting or the desired number of days after planting, in some embodiments, 9 days after planting. The experimental conetainers are infested following an infestation pattern to allow for aphid choice feeding in planta. The infested experimental plants are maintained in their growth environment until phenotyping.

The plants may be phenotyped at one or more times after infestation, for example 1 day, 4 days, 7 days or more after infestation. Measurement of one or more traits of agronomic importance is performed as follows. The height of each plant is measured, e.g., by placing the ruler on the lip of a cell and measuring the plant's height to the nearest millimeter or using an automated tool such as a Phenospex PlantEye 3D laser scanner (Phenospex B. V., Heerlen, The Netherlands). Other traits of agronomic importance may be measured either manually or using a tool such as the Phenospex PlantEye 3D laser scanner, for example the greenness of the plants and the leaf and/or above ground plant area. The mass of each plant may be measured for example via destructive sampling, e.g., by cutting the plant at the soil surface, placing the shoot in the weighing container, allowing the weight to stabilize, and autorecording the mass via the scale's software. The experimental plants may be maintained through their reproductive stages, and traits of agronomic importance such as number of flowers, number of pods and number of seeds per pod may be measured.

### Example 18. Field Assessment of Improved Plant Health of Soy Under Biotic Stress

This example describes an exemplary method by which improved plant health of endophyte treated plants were shown in a growth environment comprising Pythium or Rhizoctonia. This assay utilized soybeans and wheat.

Field trials were conducted using soybean or winter wheat seeds coated with one or more of the endophytes described herein and controls (untreated). Replicate plots were planted per endophyte or control treatment in a randomized complete block design. Each plot consisted of an approximately 7.62 m (25 ft.) by 0.76 m (2.5 ft.) row. The following growth metrics were measured: early emergence, full emergence, plant height, root weight, shoot weight, and yield.

At the end of the field trial employing endophyte treatment and control treatment plants, plants were randomly dug out from each row, kept in a plastic bag, and brought back to lab for metric measurements. For each seedling, shoot and root were separated by cutting the seedling 3 cm from the first branch of the root. The heights of the separated shoot of each plant were measured, followed by fresh shoot weight, and fresh root weight. The main root was vertically split into two halves and discoloration of xylem is scored.

Summary statistics are generated using ggplot2 package of R (R Core Team, 2016. R: A language and environment for statistical computing. R Foundation for Statistical Computing, Vienna, Austria. R-project.org/).

TABLE 10
Plant phenotypes under biotic stress of endophyte-treated plants in
field conditions values represent % change relative to controls.

| | | | | | Condition |
|---|---|---|---|---|---|
| Treatment | Metric | Value | Crop | Condition | Descriptor |
| MIC- | Early | −7.3 | Soy | Pythium | inoculated field |
| 54924 | Emergence | | | | trial |
| MIC- | Full Emergence | 3.3 | Soy | Pythium | inoculated field |
| 54924 | | | | | trial |
| MIC- | Plant Height | 4 | Soy | Pythium | inoculated field |
| 54924 | | | | | trial |
| MIC- | Root Weight | 32.4 | Soy | Pythium | inoculated field |
| 54924 | | | | | trial |
| MIC- | Shoot Weight | 12.3 | Soy | Pythium | inoculated field |
| 54924 | | | | | trial |
| MIC- | Yield | 0.5 | Soy | Pythium | inoculated field |
| 54924 | | | | | trial |
| MIC- | Early | −4.09 | Soy | Rhizoctonia | inoculated field |
| 54924 | Emergence | | | | trial |
| MIC- | Full Emergence | 38.9 | Soy | Rhizoctonia | inoculated field |
| 54924 | | | | | trial |
| MIC- | Plant Height | −4.8 | Soy | Rhizoctonia | inoculated field |
| 54924 | | | | | trial |
| MIC- | Root Weight | 0 | Soy | Rhizoctonia | inoculated field |
| 54924 | | | | | trial |
| MIC- | Shoot Weight | −2.4 | Soy | Rhizoctonia | inoculated field |
| 54924 | | | | | trial |
| MIC- | Yield | −0.8 | Soy | Rhizoctonia | inoculated field |
| 54924 | | | | | trial |

TABLE 11
Plant phenotypes under biotic stress of endophyte-
treated plants in field conditions, values represent
% change relative to untreated controls.

| | | | | | Condition |
|---|---|---|---|---|---|
| Treatment | Metric | Value | Crop | Condition | Descriptor |
| MIC- | Early | 23.4 | Winter | Pythium | inoculated field |
| 54924 | Emergence | | Wheat | | trial |
| MIC- | Full | 7.3 | Winter | Pythium | inoculated field |
| 54924 | Emergence | | Wheat | | trial |

Seven trials were conducted at locations in the United States inoculated with Fusarium graminearum in 2022.

Field trials were conducted using soybean seeds coated with MIC-54924 plus sedaxane, mefenoxam, and fludioxonil (“MIC-54924+Chem.”), seeds were also treated with sedaxane, mefenoxam, and fludioxonil without an endophyte (“Chemical Control”), and untreated controls were also prepared. Replicate plots were planted per endophyte or control treatment in a randomized complete block design. Each plot consisted of an approximately 1.5 m by 10 m row. The following growth metrics were measured: root fresh weight, full emergence, plant height, and yield.

TABLE 12
Plant phenotypes under biotic stress of endophyte-treated plants in field
conditions, values represent % change relative to untreated controls.

| | | | | | | Condition |
|---|---|---|---|---|---|---|
| Treatment | Metric | Value | Win rate | Crop | Condition | Descriptor |
| MIC-54924 + | Root Fresh | 1.2% | | Soybean | Fusarium | inoculated |
| Chem. | Weight | | | | graminearum | field trial |
| Chemical | Root Fresh | 6.1% | | Soybean | Fusarium | inoculated |
| Control | Weight | | | | graminearum | field trial |
| MIC-54924 + | Full | 21.3% | 96% | Soybean | Fusarium | inoculated |
| Chem. | Emergence | | | | graminearum | field trial |
| Chemical | Full | 23.9% | 93% | Soybean | Fusarium | inoculated |
| Control | Emergence | | | | graminearum | field trial |
| MIC-54924 + | Plant Height | 2.6% | | Soybean | Fusarium | inoculated |
| Chem. | | | | | graminearum | field trial |
| Chemical | Plant Height | 0.04% | | Soybean | Fusarium | inoculated |
| Control | | | | | graminearum | field trial |
| MIC-54924 + | Yield | 4.3% | 86% | Soybean | Fusarium | inoculated |
| Chem. | | | | | graminearum | field trial |
| Chemical | Yield | 2.5% | 71% | Soybean | Fusarium | inoculated |
| Control | | | | | graminearum | field trial |

### Example 19. Field Assessment of Improved Plant Health Under Biotic Stress

This example describes an exemplary method by which improved plant health of endophyte treated plants were shown.

### Wheat

Sixteen trials were conducted at 11 field locations under naturally occurring disease pressure in Argentina. Target diseases included Dreschera tritici repentis, Bipolaris sorokiniana, Fusarium graminearum, Rizhoctonia solani and Phytium sp.

Field trials were conducted using wheat seeds coated with MIC-54924 in water dispersed formulation at three dosages: 0.45 mL/kg seeds, 0.65 mL/kg seeds, and 1 mL/kg seeds, seeds treated with a commercial chemical fungicide (“Chemical Control”), and untreated controls. Replicate plots were planted per endophyte or control treatment in a randomized complete block design. Each plot consisted of an approximately 1.5 m by 10 m row. The following growth metrics were measured: early emergence, emergence at 10 days after emergence, full emergence, NDVI, heads per square meter prior to harvest, and yield.

TABLE 13
Plant phenotypes under biotic stress of endophyte-treated plants
in field conditions under naturally occurring disease pressure,
values represent % change relative to controls.

| | | | | | Condition |
|---|---|---|---|---|---|
| Treatment | Metric | Value | Crop | Condition | Descriptor |
| MIC-54924 | Early | 6.75 | Wheat | Naturally | 0.45 mL/kg dose |
| | emergence | | | occurring disease | |
| MIC-54924 | Early | 5.38 | Wheat | Naturally | 0.65 mL/kg dose |
| | emergence | | | occurring disease | |
| MIC-54924 | Early | −0.91 | Wheat | Naturally | 1 mL/kg dose |
| | emergence | | | occurring disease | |
| Chemical | Early | 4.92 | Wheat | Naturally | |
| control | emergence | | | occurring disease | |
| MIC-54924 | 10 days after | 9.99 | Wheat | Naturally | 0.45 mL/kg dose |
| | emergence | | | occurring disease | |
| MIC-54924 | 10 days after | 6.41 | Wheat | Naturally | 0.65 mL/kg dose |
| | emergence | | | occurring disease | |
| MIC-54924 | 10 days after | −0.41 | Wheat | Naturally | 1 mL/kg dose |
| | emergence | | | occurring disease | |
| Chemical | 10 days after | 1.92 | Wheat | Naturally | |
| control | emergence | | | occurring disease | |
| MIC-54924 | Full | 6.06 | Wheat | Naturally | 0.45 mL/kg dose |
| | emergence | | | occurring disease | |
| MIC-54924 | Full | 4.17 | Wheat | Naturally | 0.65 mL/kg dose |
| | emergence | | | occurring disease | |
| MIC-54924 | Full | −0.83 | Wheat | Naturally | 1 mL/kg dose |
| | emergence | | | occurring disease | |
| Chemical | Full | 4.85 | Wheat | Naturally | |
| control | emergence | | | occurring disease | |
| MIC-54924 | NDVI - | 2.57 | Wheat | Naturally | 0.45 mL/kg dose |
| | tillering stage | | | occurring disease | |
| MIC-54924 | NDVI - | 1.76 | Wheat | Naturally | 0.65 mL/kg dose |
| | tillering stage | | | occurring disease | |
| MIC-54924 | NDVI - | 1.62 | Wheat | Naturally | 1 mL/kg dose |
| | tillering stage | | | occurring disease | |
| Chemical | NDVI - | 0.63 | Wheat | Naturally | |
| control | tillering stage | | | occurring disease | |
| MIC-54924 | NDVI - flag | 1.02 | Wheat | Naturally | 0.45 mL/kg dose |
| | leaf stage | | | occurring disease | |
| MIC-54924 | NDVI - flag | 2.47 | Wheat | Naturally | 0.65 mL/kg dose |
| | leaf stage | | | occurring disease | |
| MIC-54924 | NDVI - flag | 1.12 | Wheat | Naturally | 1 mL/kg dose |
| | leaf stage | | | occurring disease | |
| Chemical | NDVI - flag | 3.33 | Wheat | Naturally | |
| control | leaf stage | | | occurring disease | |

TABLE 14
Plant phenotypes under biotic stress of endophyte-treated plants
in field conditions under naturally occurring disease pressure,
values number of heads of wheat per square meter.

| | | | | | Treatment |
|---|---|---|---|---|---|
| Treatment | Metric | Value | Crop | Condition | Descriptor |
| MIC-54924 | Heads per | 346.00 | Wheat | Naturally | 0.45 mL/kg dose |
| | square meter | | | occurring disease | |
| MIC-54924 | Heads per | 342.15 | Wheat | Naturally | 0.65 mL/kg dose |
| | square meter | | | occurring disease | |
| MIC-54924 | Heads per | 341.16 | Wheat | Naturally | 1 mL/kg dose |
| | square meter | | | occurring disease | |
| Chemical control | Heads per | 346.94 | Wheat | Naturally | |
| | square meter | | | occurring disease | |
| Untreated | Heads per | 334.18 | Wheat | Naturally | |
| | square meter | | | occurring disease | |

TABLE 15
Plant phenotypes under biotic stress of endophyte-treated plants
in field conditions under naturally occurring disease pressure,
values represent % change relative to controls.

| | | | | | | Condition |
|---|---|---|---|---|---|---|
| Treatment | Metric | Value | Win rate | Crop | Condition | Descriptor |
| MIC-54924 | Yield | 1.71 | 68% | Wheat | Naturally | 0.45 |
| | | | | | occurring | mL/kg |
| | | | | | disease | dose |
| MIC-54924 | Yield | 5.11 | 83% | Wheat | Naturally | 0.65 |
| | | | | | occurring | mL/kg |
| | | | | | disease | dose |
| MIC-54924 | Yield | 1.56 | 51% | Wheat | Naturally | 1 |
| | | | | | occurring | mL/kg |
| | | | | | disease | dose |
| Chemical | Yield | 4.33 | 68% | Wheat | Naturally | |
| control | | | | | occurring | |
| | | | | | disease | |

Fifteen winter wheat seedling disease trials were conducted in the USA in 2022; 5 trials were inoculated with Pythium, 5 were inoculated with Rhizoctonia, and 5 were inoculated with Fusarium.

Field trials were conducted using wheat seeds coated with MIC-54924 in water dispersed formulation and untreated controls. Replicate plots were planted per endophyte or control treatment in a randomized complete block design. In the Fusarium trials, Fusarium graminearum inoculum was applied in-furrow and data were collected across 14 data points per treatement. In the Pythium trials, Pythium ultimum inoculum was applied in-furrow and data was collected across 22 data points per treatment. The following growth metrics were measured: early emergence (0-2 days after beginning of emergence) and full emergence (approximately 10 days after the early emergence count). Sites were classified as stressed when the chemical control provided significant improvement or >5% improvement in full emergence relative to the untreated control.

MIC-54924 treated seeds had an average of 1.94% increase in percent full emergence relative to untreated controls in the Fusarium inoculated trials. MIC-54924 treated seeds had an average of 7.31% increase in percent full emergence relative to untreated controls in the Pythium inoculated trials, this translates to an additional 56,917 plants per acre relative to untreated controls with a win rate of 68%.

### Soybeans

Eight trials were conducted at locations in Argentina under natural disease pressure in 2020 and 2021. The locations had low incidence of fungal pathogen in 2020.

Field trials were conducted using soybean seeds: coated with MIC-54924 in water dispersed formulation at three dosages: 0.50 mL/kg seeds, 1.3 mL/kg seeds, and 2 mL/kg seeds, seeds treated with a commercial chemical fungicide (“Chemical Control”), and untreated controls. Replicate plots were planted per endophyte or control treatment in a randomized complete block design. Each plot consisted of an approximately 1.5 m by 10 m row. The following growth metrics were measured: early emergence, full emergence, and yield.

TABLE 16
Plant phenotypes under biotic stress of endophyte-treated plants
in field conditions with low incidence of fungal pathogen pressure
(2H2020), values represent % change relative to controls.

| | | | | | Condition |
|---|---|---|---|---|---|
| Treatment | Metric | Value | Crop | Condition | Descriptor |
| MIC-54924 | Early | 1.38 | Soybean | Naturally | 0.50 mL/kg |
| | emergence | | | occurring | dose |
| | | | | disease | |
| MIC-54924 | Early | −2.11 | Soybean | Naturally | 1.3 mL/kg |
| | emergence | | | occurring | dose |
| | | | | disease | |
| MIC-54924 | Early | −4.38 | Soybean | Naturally | 2 mL/kg dose |
| | emergence | | | occurring | |
| | | | | disease | |
| Chemical | Early | −2.35 | Soybean | Naturally | |
| Control | emergence | | | occurring | |
| | | | | disease | |
| MIC-54924 | Full | 1.0 | Soybean | Naturally | 0.50 mL/kg |
| | emergence | | | occurring | dose |
| | | | | disease | |
| MIC-54924 | Full | 2.34 | Soybean | Naturally | 1.3 mL/kg |
| | emergence | | | occurring | dose |
| | | | | disease | |
| MIC-54924 | Full | 0.23 | Soybean | Naturally | 2 mL/kg dose |
| | emergence | | | occurring | |
| | | | | disease | |
| Chemical | Full | 3.13 | Soybean | Naturally | |
| Control | emergence | | | occurring | |
| | | | | disease | |
| MIC-54924 | Yield | 1.41 | Soybean | Naturally | 0.50 mL/kg |
| | | | | occurring | dose |
| | | | | disease | |
| MIC-54924 | Yield | 0.14 | Soybean | Naturally | 1.3 mL/kg |
| | | | | occurring | dose |
| | | | | disease | |
| MIC-54924 | Yield | 1.58 | Soybean | Naturally | 2 mL/kg dose |
| | | | | occurring | |
| | | | | disease | |
| Chemical | Yield | 1.24 | Soybean | Naturally | |
| Control | | | | occurring | |
| | | | | disease | |

TABLE 17
Plant phenotypes under biotic stress of endophyte-
treated plants in field conditions (2H2021), values
represent % change relative to controls.

| | | | | | Condition |
|---|---|---|---|---|---|
| Treatment | Metric | Value | Crop | Condition | Descriptor |
| MIC-54924 | Early | 7.20 | Soybean | Naturally | 0.50 mL/kg |
| | emergence | | | occurring | dose |
| | | | | disease | |
| MIC-54924 | Early | 2.06 | Soybean | Naturally | 1.3 mL/kg |
| | emergence | | | occurring | dose |
| | | | | disease | |
| MIC-54924 | Early | −0.43 | Soybean | Naturally | 2 mL/kg dose |
| | emergence | | | occurring | |
| | | | | disease | |
| Chemical | Early | −0.47 | Soybean | Naturally | |
| Control | emergence | | | occurring | |
| | | | | disease | |
| MIC-54924 | Full | −0.61 | Soybean | Naturally | 0.50 mL/kg |
| | emergence | | | occurring | dose |
| | | | | disease | |
| MIC-54924 | Full | 2.22 | Soybean | Naturally | 1.3 mL/kg |
| | emergence | | | occurring | dose |
| | | | | disease | |
| MIC-54924 | Full | 4.49 | Soybean | Naturally | 2 mL/kg dose |
| | emergence | | | occurring | |
| | | | | disease | |
| Chemical | Full | −0.33 | Soybean | Naturally | |
| Control | emergence | | | occurring | |
| | | | | disease | |
| MIC-54924 | Yield | 7.16 | Soybean | Naturally | 0.50 mL/kg |
| | | | | occurring | dose |
| | | | | disease | |
| MIC-54924 | Yield | 5.41 | Soybean | Naturally | 1.3 mL/kg |
| | | | | occurring | dose |
| | | | | disease | |
| MIC-54924 | Yield | 4.07 | Soybean | Naturally | 2 mL/kg dose |
| | | | | occurring | |
| | | | | disease | |
| Chemical | Yield | 4.96 | Soybean | Naturally | |
| Control | | | | occurring | |
| | | | | disease | |

TABLE 18
Plant phenotypes under biotic stress of endophyte-treated plants
in field conditions under naturally occurring disease pressure,
values are average yield across all trials in kg/hectare.

| | | | | | Treatement |
|---|---|---|---|---|---|
| Treatment | Metric | Value | Crop | Condition | Descriptor |
| MIC-54924 | Yield | 261.55 | Soybean | Naturally | 0.5 mL/kg dose |
| | | | | occurring disease | |
| MIC-54924 | Yield | 197.485 | Soybean | Naturally | 1.3 mL/kg dose |
| | | | | occurring disease | |
| MIC-54924 | Yield | 148.69 | Soybean | Naturally | 2 mL/kg dose |
| | | | | occurring disease | |
| Chemical control | Yield | 181.15 | Soybean | Naturally | |
| | | | | occurring disease | |
| Untreated | Yield | 145.69 | Soybean | Naturally | |
| | | | | occurring disease | |

Sixteen trials were conducted at locations in Brazil in fields inoculated with Fusarium solani. Sixteen trials were also conducted in fields inoculated with Macrophomina phaseolina. Sixteen trials were also conducted in fields inoculated with Rhizoctonia solani.

Field trials were conducted using soybean seeds coated with MIC-54924 in water dispersed formulation at three dosages: 0.50 mL/kg seeds, 1 mL/kg seeds, 1.3 mL/kg seeds, and 2 mL/kg seeds, seeds treated with a commercial chemical fungicide (“Chemical Control”), and untreated controls. Replicate plots (4) were planted per endophyte or control treatment in a randomized complete block design. Each plot consisted of 24 square meters. The following growth metrics were measured: early emergence and full emergence.

Each dosage produced significant increases in early and full emergence relative to untreated plants in field conditions comprising Fusarium solani stress, and in field conditions comprising Macrophomina phaseolina stress. Each dosage produced significant increases in early emergence relative to untreated plants in field conditions comprising Rhizoctonia solani stress.

### Corn

Trials were conducted across 8 locations that were under naturally occurring disease pressure, however, these locations had low incidence of fungal pathogen. Two locations were inoculated with ground sorghum seed infested with F. graminearum and F. verticilioides which was was applied in furrow at planting time.

Field trials were conducted using corn seeds coated with MIC-54924 in water dispersed formulation at three dosages: 0.55 mL/kg seeds, 1.3 mL/kg seeds, and 2 mL/kg seeds, and untreated controls. Replicate plots were planted per endophyte or control treatment in a randomized complete block design. Each plot consisted of an approximately 1.5 m by 10 m row. The following growth metrics were measured: early emergence, full emergence, and yield.

TABLE 19
Plant phenotypes under biotic stress of endophyte-treated plants
in field conditions with low incidence of fungal pathogen pressure,
values represent % change relative to controls.

| | | | | | Condition |
|---|---|---|---|---|---|
| Treatment | Metric | Value | Crop | Condition | Descriptor |
| MIC- | Early | −0.49 | Corn | Fusarium | 0.55 mL/kg |
| 54924 | emergence | | | inoculated | dose |
| MIC- | Early | −2.49 | Corn | Fusarium | 1.3 mL/kg |
| 54924 | emergence | | | inoculated | dose |
| MIC- | Early | −5.37 | Corn | Fusarium | 2 mL/kg dose |
| 54924 | emergence | | | inoculated | |
| MIC- | Full | 4.48 | Corn | Fusarium | 0.55 mL/kg |
| 54924 | emergence | | | inoculated | dose |
| MIC- | Full | −5.37 | Corn | Fusarium | 1.3 mL/kg |
| 54924 | emergence | | | inoculated | dose |
| MIC- | Full | −3.41 | Corn | Fusarium | 2 mL/kg dose |
| 54924 | emergence | | | inoculated | |
| MIC- | Yield | 7.3 | Corn | Fusarium | 0.55 mL/kg |
| 54924 | | | | inoculated | dose |
| MIC- | Yield | 10.3 | Corn | Fusarium | 1.3 mL/kg |
| 54924 | | | | inoculated | dose |
| MIC- | Yield | 11.1 | Corn | Fusarium | 2 mL/kg dose |
| 54924 | | | | inoculated | |
| MIC- | Yield | 1.03 | Corn | Low incidence | 0.55 mL/kg |
| 54924 | | | | natural disease | dose |
| MIC- | Yield | 4.03 | Corn | Low incidence | 1.3 mL/kg |
| 54924 | | | | natural disease | dose |
| MIC- | Yield | 4.87 | Corn | Low incidence | 2 mL/kg dose |
| 54924 | | | | natural disease | |

### Example 20. Field Assessment of Improved Plant Health

This example describes an exemplary method by which improved plant health of endophyte treated plants may be shown.

Field trials are conducted using seeds (e.g. cotton, soy, corn, wheat, etc. optionally chemically treated) coated with one or more of the endophytes described herein and formulation control (lacking the one or more heterologously disposed endophytes) and untreated controls (lacking formulation and the one or more heterologously disposed endophyte) as described herein. The following growth metrics are measured: percent emergence at 14 days post planting, standing count at 28 and 45 days post planting, plant vigor at 14, 28, and 45 days post planting, plant height at 45 days post planting, fresh shoot weight, fresh root weight, disease rating at a 0-3 scale (3 denotes strong disease symptoms) using the split-root scoring system at 45 days post planting, nematode count at 45 days post planting, and yield parameters.

Summary statistics are generated using ggplot2 package of R (R Core Team, 2016. R: A language and environment for statistical computing. R Foundation for Statistical Computing, Vienna, Austria. R-project.org/).

### Example 21. Method of Determining Seed Nutritional Quality Trait Component: Fat

Seed samples from harvested plants are obtained as described herein. Analysis of fat is conducted on replicate samples according to the Association of Official Agricultural Chemists Reference Method AOAC 920.39, of the Official Methods of Analysis of AOAC International, 20th Edition (2016), herein incorporated by reference in its entirety. Samples are weighed onto filter paper, dried, and extracted in hot hexane for 4 hrs. using a Soxlhet system. Oil is recovered in pre-weighed glassware, and % fat is measured gravimetrically. Mean percent changes between the treatment (endophyte-treated seed) and control (seed treated with the formulation but no endophyte) are calculated.

### Example 22. Method of Determining Seed Nutritional Quality Trait Component: Ash

Seed samples from harvested plants are obtained as described herein. Analysis of ash is conducted on replicate samples according to the Association of Official Agricultural Chemists Reference Method AOAC 920.39, of the Official Methods of Analysis of AOAC International, 20th Edition (2016). Samples are weighed into pre-weighed crucibles, and ashed in a furnace at 600° C. for 3 hr. Weight loss on ashing is calculated as % ash. Mean percent changes between the treatment (one or more heterologously disposed endophytes) and control (lacking the one or more heterologously disposed endophytes) with the formulation but no endophyte are calculated

### Example 23. Method of Determining Seed Nutritional Quality Trait Component: Fiber

Seed samples from harvested plants are obtained as described herein. Analysis of fiber is conducted on replicate samples according to the Association of Official Agricultural Chemists Reference Method AOAC 920.39, of the Official Methods of Analysis of AOAC International, 20th Edition (2016). Samples are weighed into filter paper, defatted and dried, and hydrolyzed first in acid, then in alkali solution. The recovered portion is dried, weighed, ashed at 600° C., and weighed again. The loss on ashing is calculated as % Fiber. Mean percent changes between the treatment (one or more heterologously disposed endophytes) and control (lacking the one or more heterologously disposed endophytes) with the formulation but no endophyte are calculated.

### Example 24. Method of Determining Seed Nutritional Quality Trait Component: Moisture

Seed samples from harvested plants are obtained as described herein. Analysis of moisture is conducted on replicate samples according to the Association of Official Agricultural Chemists Reference Method AOAC 920.39, of the Official Methods of Analysis of AOAC International, 20th Edition (2016). Samples are weighed into pre-weighed aluminum dishes and dried at 135° C. for 2 hrs. Weight loss on drying is calculated as % Moisture. Mean percent changes between the treatment (one or more heterologously disposed endophytes) and control (lacking the one or more heterologously disposed endophytes) with the formulation but no endophyte are calculated.

### Example 25. Method of Determining Seed Nutritional Quality Trait Component: Protein

Seed samples from harvested plants are obtained as described herein. Analysis of protein is conducted on replicate samples according to the Association of Official Agricultural Chemists Reference Method AOAC 920.39, of the Official Methods of Analysis of AOAC International, 20th Edition (2016). Samples are combusted and nitrogen gas is measured using a combustion nitrogen analyzer (Dumas). Nitrogen is multiplied by 6.25 to calculate % Protein. Mean percent changes between the treatment (one or more heterologously disposed endophytes) and control (lacking the one or more heterologously disposed endophytes) with the formulation but no endophyte) are calculated.

### Example 26. Method of Determining Seed Nutritional Quality Trait Component: Carbohydrate

Seed samples from harvested plants are obtained as described herein. Analysis of carbohydrate is determined for replicate samples as a calculation according to the following formula: Total Carbohydrate=100%-% (Protein+Ash+Fat+Moisture+Fiber), where % Protein is determined according to the method described herein, % Ash is determined according to the method described herein, % Fat is determined according to the method described herein, % Moisture is determined according to the method described herein, and % Fiber is determined according to the method described herein. Mean percent changes between the treatment (one or more heterologously disposed endophytes) and control (lacking the one or more heterologously disposed endophytes) are calculated.

### Example 27. Method of Determining Seed Nutritional Quality Trait Component: Calories

Seed samples from harvested plants are obtained as described herein. Analysis of Calories is determined for replicate samples as a calculation according to the following formula: Total Calories=(Calories from protein)+ (Calories from carbohydrate)+Calories from fat), where Calories from protein are calculated as 4 Calories per gram of protein (as determined according to the method described herein), Calories from carbohydrate are calculated as 4 Calories per gram of carbohydrate (as determined according to the method described herein), and Calories from fat are calculated as 9 Calories per gram of fat (as determined according to the method described herein). Mean percent changes between the treatment (one or more heterologously disposed endophytes) and control (lacking the one or more heterologously disposed endophytes) are calculated.

### Example 28. Blotter Paper Assessment of Improved Plant Health Under Biotic Stress (Cercospora kikuchii)

This example describes an exemplary method by which improved plant health of endophyte treated plants may be shown in a growth environment comprising the crop pathogens: Cercospora kikuchii, Fusarium spp., and Phomopsis sp.

Assays were conducted using soybean seeds coated with MIC-54924 and untreated controls (lacking formulation and the one or more heterologously disposed endophyte). Three doses of MIC-54924 in water dispersed formulation were applied: 0.5 mL inoculant per kg seed, 1.3 inoculant per kg seed, 2 inoculant per kg seed. Treated soybean seeds and untreated controls were placed on damp blotter paper inoculated with the fungal pathogen and incubated at 24 degrees Celsius for 8 days with 12 hours of light/dark. Eight replicated sets of 50 seeds per treatment or control were prepared. Seeds were scored for visual incidence of pathogen infection. Treatment with MIC-54924 at 1.3 ml inoculant/kg seed resulted in a significant reduction of incidence of Cercospora kikuchii on the treated seeds compared to untreated controls. Treatment with MIC-54924 at 0.5 mL/kg, 1.3 mL/kg, and 2 mL/kg resulted in a significant reduction of incidence of Fusarium spp. on the treated seeds compared to untreated controls. Treatment with MIC-54924 at all resulted in reduction of incidence of Phomopsis sp. on the treated seeds compared to untreated controls, with especially significant results in the 0.5 mL/kg, 1.3 mL/kg dosages.

TABLE 20
Observed incidence of C. kikuchi in blotter paper assessment

| Treatment | Average % incidence C. kikuchi |
|---|---|
| MIC-54924 @ 0.5 ml/kg | 11.5 |
| MIC-54924 @ 2 ml/kg | 11 |
| Untreated | 11 |
| MIC-54924 @ 1.3 ml/kg | 5.25 |

### Example 29. Analysis of Antifungal Peptide W1

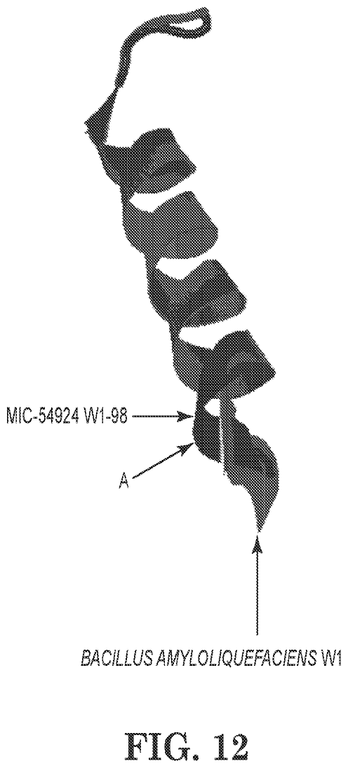

The structures of sequence variants of the antifungal peptide W1 identified in MIC-54924 (SEQ IDs. 98 and 99) were compared to antifungal protein W1 identified in marine Bacillus amyloliquefaciens (Wen et al. Front. Microbiol., 2022 Volume 13). A predicted protein structures was generated for Bacillus amyloliquefaciens W1 and MIC-54924 W1 SEQ ID. 98 (“MIC-54924 W1-98”) and MIC-54924 W1 SEQ ID. 99 (“MIC-54924 W1-99”) using PEP-FOLD3 (Lamiable et al. Nucleic Acids Res. 2016 Jul. 8; 44 (Web Server issue): W449-W454). TM-SCORE is a scoring function to assess the similarity of protein structures (Y. Zhang, J. Skolnick, Proteins, 57:702-710 (2004). A TM-score greater than 0 and less than 0.17 indicates random structural similarity, and a TM-score of greater than 0.5 and less than 1 indicates structures expected to have similar structure (Yang Zhang and Jeffrey Skolnick, Proteins 2004 57:702-710). The TM-SCORE of MIC-54924 W1-99 and Bacillus amyloliquefaciens W1 was TM-score was 0.49. Comparison of predicted structures MIC-54924 W1-99 and Bacillus amyloliquefaciens W1 (FIG. 11) and MIC-54924 W1-98 and Bacillus amyloliquefaciens W1 (FIG. 12) shows the presence of a kink in the MIC-54924 W1-98 and MIC-54924 W1-99 structures at the amino terminus of the structure, indicated by the arrow labeled “A” in FIG. 11 and FIG. 12.

TABLE 21
Amino acid variants of MIC-54924 W1.

| SEQID | Variant | Sequence |
|---|---|---|
| 100 | W1 peptide (SEQ ID: 99), L4F | MGVFQNIWPLLLMFVIFYFLLIRPQ |
| 101 | W1 peptide (SEQ ID: 99), L4F N6Q | MGVFQQIWPLLLMFVIFYFLLIRPQ |
| 102 | W1 peptide (SEQ ID: 99), L4F Q5N | MGVFNNIWPLLLMFVIFYFLLIRPQ |
| 103 | W1 peptide (SEQ ID: 99), L4F Q5N N6Q | MGVFNQIWPLLLMFVIFYFLLIRPQ |
| 104 | W1 peptide (SEQ ID: 99), L4F Q5N N6Q W8Y | MGVFNQIYPLLLMFVIFYFLLIRPQ |
| 105 | W1 peptide (SEQ ID: 99), L4F Q5N W8Y | MGVFNNIYPLLLMFVIFYFLLIRPQ |
| 106 | W1 peptide (SEQ ID: 99), L4F W8Y | MGVFQNIYPLLLMFVIFYFLLIRPQ |
| 107 | W1 peptide (SEQ ID: 99), L4M | MGVMQNIWPLLLMFVIFYFLLIRPQ |
| 108 | W1 peptide (SEQ ID: 99), L4M N6Q | MGVMQQIWPLLLMFVIFYFLLIRPQ |
| 109 | W1 peptide (SEQ ID: 99), L4M Q5N | MGVMNNIWPLLLMFVIFYFLLIRPQ |
| 110 | W1 peptide (SEQ ID: 99), L4M Q5N N6Q | MGVMNQIWPLLLMFVIFYFLLIRPQ |
| 111 | W1 peptide (SEQ ID: 99), L4M Q5N N6Q W8Y | MGVMNQIYPLLLMFVIFYFLLIRPQ |
| 112 | W1 peptide (SEQ ID: 99), L4M Q5N W8Y | MGVMNNIYPLLLMFVIFYFLLIRPQ |
| 113 | W1 peptide (SEQ ID: 99), L4M W8Y | MGVMQNIYPLLLMFVIFYFLLIRPQ |
| 114 | W1 peptide (SEQ ID: 99), L4V | MGVVQNIWPLLLMFVIFYFLLIRPQ |
| 115 | W1 peptide (SEQ ID: 99), L4V N6Q | MGVVQQIWPLLLMFVIFYFLLIRPQ |
| 116 | W1 peptide (SEQ ID: 99), L4V Q5N | MGVVNNIWPLLLMFVIFYFLLIRPQ |
| 117 | W1 peptide (SEQ ID: 99), L4V Q5N N6Q | MGVVNQIWPLLLMFVIFYFLLIRPQ |
| 118 | W1 peptide (SEQ ID: 99), L4V Q5N N6Q W8Y | MGVVNQIYPLLLMFVIFYFLLIRPQ |
| 119 | W1 peptide (SEQ ID: 99), L4V Q5N W8Y | MGVVNNIYPLLLMFVIFYFLLIRPQ |
| 120 | W1 peptide (SEQ ID: 99), L4V W8Y | MGVVQNIYPLLLMFVIFYFLLIRPQ |
| 121 | W1 peptide (SEQ ID: 99), N6Q | MGVLQQIWPLLLMFVIFYFLLIRPQ |
| 122 | W1 peptide (SEQ ID: 99), N6Q W8Y | MGVLQQIYPLLLMFVIFYFLLIRPQ |
| 123 | W1 peptide (SEQ ID: 99), Q5N | MGVLNNIWPLLLMFVIFYFLLIRPQ |
| 124 | W1 peptide (SEQ ID: 99), Q5N N6Q | MGVLNQIWPLLLMFVIFYFLLIRPQ |
| 125 | W1 peptide (SEQ ID: 99), Q5N N6Q W8Y | MGVLNQIYPLLLMFVIFYFLLIRPQ |
| 126 | W1 peptide (SEQ ID: 99), Q5N W8Y | MGVLNNIYPLLLMFVIFYFLLIRPQ |
| 127 | W1 peptide (SEQ ID: 99), W8Y | MGVLQNIYPLLLMFVIFYFLLIRPQ |
| 128 | W1 peptide (SEQ ID: 98), Q1N | NNIWPLLLMFVIFYFLLIRPQ |
| 129 | W1 peptide (SEQ ID: 98), Q1N W4Y | NNIYPLLLMFVIFYFLLIRPQ |
| 130 | W1 peptide (SEQ ID: 98), Q1N N2Q | NQIWPLLLMFVIFYFLLIRPQ |
| 131 | W1 peptide (SEQ ID: 98), Q1N N2Q W3Y | NQIYPLLLMFVIFYFLLIRPQ |
| 132 | W1 peptide (SEQ ID: 98), W3Y | QNIYPLLLMFVIFYFLLIRPQ |
| 133 | W1 peptide (SEQ ID: 98), N2Q | QQIWPLLLMFVIFYFLLIRPQ |
| 134 | W1 peptide (SEQ ID: 98), N2Q W3Y | QQIYPLLLMFVIFYFLLIRPQ |

Having illustrated and described the principles of the present invention, it should be apparent to persons skilled in the art that the invention can be modified in arrangement and detail without departing from such principles. It is to be understood that while the invention has been described in conjunction with the detailed description thereof, the foregoing description is intended to illustrate and not limit the scope of the invention, which is defined by the scope of the appended claims. Other embodiments, advantages, and modifications are within the scope of the following claims.

1. A synthetic composition comprising an endophyte, wherein the synthetic composition comprises an endophyte heterologously disposed to a plant element, wherein the endophyte comprises at least one polynucleotide sequence that is at least 97% identical to one or more of SEQ ID NOs. 5-51, or combinations thereof.

2. The synthetic composition of claim 1, wherein the endophyte comprises:
(a) at least one polynucleotide sequence that is 100% identical to SEQ ID NO. 5, and at least one polynucleotide sequence that is at least 97% identical to one or more of SEQ ID NOs. 6-51;
(b) at least one polynucleotide sequence that is at least 97% identical to each of SEQ ID NOs. 5-51;
(c) at least one polynucleotide sequence that is at least 100% identical to each of SEQ ID NOs. 5-51;
(d) one or more a polynucleotide sequences that are 100% identical to a subregion within one or more of SEQ ID NOs. 5-51, wherein the subregion is a 100, 200, 300, 400, 500, 600, 700, or 800 nucleotides in length;
(e) at least one polynucleotide sequence that is:
(i) 100% identical to SEQ ID NO. 5:
(ii) at least 97% identical to one or more of SEQ ID NOs. 6, 7, 8, 9, 10, 11, 12, 13, or 14;
(iii) at least 97% identical to one or more of SEQ ID NOs. 15, 16, 17, 18, 19, 20, 21, or 22;
(iv) at least 97% identical to one or more of SEQ ID NOs. 23, 24, 25, 26, or 27;
(v) at least 97% identical to one or more of SEQ ID NOs. 28, 29, 30, 31, 32, 33, 34, 35, 36, 37, 38, or 39;
(vi) at least 97% identical to one or more of SEQ ID NOs. 42, 43, 44, 45, 46, 47, 48, 49, 50, or 51;
(vii) at least 97% identical SEQ ID NO. 40; and
(viii) at least 97% identical SEQ ID NO. 41;
(f) at least one polynucleotide sequence that is at least 97% identical to one or more of SEQ ID NOs. 6, 7, 8, 9, 10, 11, 12, 13, 14, or combinations thereof;
(g) at least one polynucleotide sequence that is at least 97% identical to one or more of SEQ ID NOs. 15, 16, 17, 18, 19, 20, 21, 22, or combinations thereof;
(h) at least one polynucleotide sequence that is at least 97% identical to one or more of SEQ ID NOs. 23, 24, 25, 26, 27, or combinations thereof;
(i) at least one polynucleotide sequence that is at least 97% identical to one or more of SEQ ID NOs. 28, 29, 30, 31, 32, 33, 34, 35, 36, 37, 38, 39, or combinations thereof;
(i) at least one polynucleotide sequence that is at least 97% identical to one or more of SEQ ID NOs. 42, 43, 44, 45, 46, 47, 48, 49, 50, 51, or combinations thereof;
(k) at least one polynucleotide sequence that is at least 97% to SEQ ID NO. 40; or
(l) at least one polynucleotide sequence that is at least 97% to SEQ ID NO. 41.

3.-12. (canceled)

13. The synthetic composition of claim 1, wherein the endophyte is a modified endophyte comprises at least one artificially introduced genetic modification to SEQ ID NO. 40, and wherein the modified endophyte is capable of producing a peptide having an amino acid sequence selected from SEQ ID NOs. 100-134.

14. (canceled)

15. (canceled)

16. The synthetic composition of claim 1, wherein the endophyte comprises;
(a) one or more genes encoding a protein whose amino acid sequence comprises an amino acid sequence selected from SEQ ID NOs. 52-134; or
(b) one or more genes encoding a protein whose amino acid sequence is selected from SEQ ID NOs. 97-134.

17. (canceled)

18. The synthetic composition of claim 1, wherein the endophyte is present:
(a) in an amount capable of improving a trait of agronomic importance in a plant;
(b) in the synthetic composition in a concentration of at least 1.0E+2 CFU/g;
(c) in the synthetic composition in a concentration of at least 1.0E+3 CFU/g;
(d) in the synthetic composition in a concentration of at least 1.0E+4 CFU/g;
(e) in the synthetic composition in a concentration of at least 1.0E+5 CFU/g;
(f) in the synthetic composition in a concentration of at least 1.0E+6 CFU/g;
(g) in the synthetic composition in a concentration of at least 1.0E+7 CFU/g;
(h) in the synthetic composition in a concentration of at least 1.0E+8 CFU/g;
(i) in the synthetic composition in a concentration of at least 1.0E+9 CFU/g;
(i) in the synthetic composition in a concentration between 1.0E+5 CFU/g and 1.0E+10 CFU/g; or
(k) in the synthetic composition in a concentration between 1.0E+6 CFU/g and 1.0E+9 CFU/g.

19. The synthetic composition of claim 18, wherein the trait of agronomic importance is selected from the group consisting of yield, root fresh weight, shoot fresh weight, biotic stress tolerance, drought tolerance, and combinations thereof.

20. The synthetic composition of claim 18, wherein the trait of agronomic importance is biotic stress tolerance, and wherein the biotic stress tolerance:
(a) is a growth environment comprising a fungal pathogen; or
(b) is shown by increased emergence, increased biomass, increased NDVI, decreased pathogen incidence, decreased necrosis, decreased chlorosis, decreased area of necrotic tissue, decreased area of chlorotic tissue, or increased yield.

21. (canceled)

22. (canceled)

23. The synthetic composition of claim 20, wherein the pathogen is one or more pathogens of the genus Pythium, Rhizoctonia, Fusarium, Cercospora, Colletotrichum, Dreschslera, Corynespora, Diaporthe, or Macrophomina.

24.-33. (canceled)

34. The synthetic composition of claim 1, further comprising a plant element.

35. The synthetic composition of claim 34, wherein the plant element is:
(a) a monocot;
(b) is a dicot; or
(c) is a whole plant, seedling, meristematic tissue, ground tissue, vascular tissue, dermal tissue, seed, leaf, root, shoot, stem, flower, fruit, stolon, bulb, tuber, corm, keikis, or bud.

36. The synthetic composition of claim 35, wherein;
(a) the monocot is a cereal and wherein the cereal is selected from the group consisting of wheat, rice, barley, buckwheat, rye, millet, oats, corn, sorghum, triticale, and spelt;
(b) the dicot is selected from the group consisting of cotton, tomato, lettuce, peppers, cucumber, endive, melon, potato, and squash; or
(c) the dicot is a legume, wherein the legume is soy, peas, or beans.

37.-42. (canceled)

43. The synthetic composition of claim 1, further comprising:
(a) one or more endophytes; or
(b) one or more of a surfactant, a buffer, a tackifier, a microbial stabilizer, a fungicide, an anticomplex agent, an herbicide, a nematicide, a fertilizer, an insecticide, a plant growth regulator, a rodenticide, a desiccant, a nutrient, an excipient, a wetting agent, a salt, and a polymer.

44. (canceled)

45. (canceled)

46. The synthetic composition of claim 1, wherein:
(a) the endophyte is dead; or
(b) wherein the endophyte is produced in a low nutrient growth media.

47. (canceled)

48. The synthetic composition of claim 43, wherein:
(a) the fertilizer is granular or liquid in form and comprises one or more of nitrogen, phosphorous, potassium, sulfur, magnesium, silica, iron, zinc, manganese, copper, boron, or fulvic acid; or
(b) the fungicide comprises one or more of fludioxonil, sedaxane, or mefenoxam.

49. A method of treating an agricultural plant, comprising heterologously disposing a plant element or planting media with a synthetic composition comprising an endophyte, wherein the endophyte comprises at least one polynucleotide sequence that is at least 97% identical to one or more of SEQ ID NOs. 5-51, or combinations thereof.

50. The method of claim 49, wherein the endophyte comprises;
(a) at least one polynucleotide sequence that is 100% identical to SEQ ID NO. 5, and at least one polynucleotide sequence that is at least 97% identical to one or more of SEQ ID NOs. 6-51;
(b) at least one polynucleotide sequence that is at least 97% identical to each of SEQ ID NOs. 5-51;
(c) at least one polynucleotide sequence that is at least 100% identical to each of SEQ ID NOs. 5-51;
(d) one or more a polynucleotide sequences that are 100% identical to a subregion within one or more of SEQ ID NOs. 5-51, wherein the subregion is a 100, 200, 300, 400, 500, 600, 700, or 800 nucleotides in length;
(e) at least one polynucleotide sequence that is:
(i) 100% identical to SEQ ID NO. 5, and at least one polynucleotide sequence that is at least 97% identical to one or more of SEQ ID NOs. 6, 7, 8, 9, 10, 11, 12, 13, or 14;
(ii) at least 97% identical to one or more of SEQ ID NOs. 15, 16, 17, 18, 19, 20, 21, or 22;
(iii) at least 97% identical to one or more of SEQ ID NOs. 23, 24, 25, 26, or 27;
(iv) at least 97% identical to one or more of SEQ ID NOs. 28, 29, 30, 31, 32, 33, 34, 35, 36, 37, 38, or 39;
(v) at least 97% identical to one or more of SEQ ID NOs. 42, 43, 44, 45, 46, 47, 48, 49, 50, or 51;
(vi) at least 97% identical SEQ ID NO. 40; and
(vii) at least 97% identical SEQ ID NO. 41;
(f) at least one polynucleotide sequence that is at least 97% identical to one or more of SEQ ID NOs. 6, 7, 8, 9, 10, 11, 12, 13, 14, or combinations thereof;
(g) at least one polynucleotide sequence that is at least 97% identical to one or more of SEQ ID NOs. 15, 16, 17, 18, 19, 20, 21, 22, or combinations thereof;
(h) at least one polynucleotide sequence that is at least 97% identical to one or more of SEQ ID NOs. 23, 24, 25, 26, 27, or combinations thereof;
(i) at least one polynucleotide sequence that is at least 97% identical to one or more of SEQ ID NOs. 28, 29, 30, 31, 32, 33, 34, 35, 36, 37, 38, 39, or combinations thereof;
(i) at least one polynucleotide sequence that is at least 97% identical to one or more of SEQ ID NOs. 42, 43, 44, 45, 46, 47, 48, 49, 50, 51, or combinations thereof;
(k) at least one polynucleotide sequence that is at least 97% to SEQ ID NO. 40; or
(l) at least one polynucleotide sequence that is at least 97% to SEQ ID NO. 41.

51.-60. (canceled)

61. The method of claim 49, wherein the endophyte is a modified endophyte comprising at least one artificially introduced genetic modification to SEQ ID NO. 40, and wherein the modified endophyte is capable of producing a peptide having an amino acid sequence selected from SEQ ID NOs. 100-134.

62. (canceled)

63. (canceled)

64. The method of claim 49, wherein the endophyte comprises:
(a) one or more genes encoding a protein whose amino acid sequence comprises an amino acid sequence selected from SEQ ID NOs. 52-134; or
(b) one or more genes encoding a protein whose amino acid sequence is selected from SEQ ID NOs. 97-134.

65. (canceled)

66. The method of claim 49, wherein the endophyte is present;
(a) in an amount capable of improving a trait of agronomic importance in a plant;
(b) in the synthetic composition in a concentration of at least 1.0E+2 CFU/g;
(c) in the synthetic composition in a concentration of at least 1.0E+3 CFU/g;
(d) in the synthetic composition in a concentration of at least 1.0E+4 CFU/g;
(e) present in the synthetic composition in a concentration of at least 1.0E+5 CFU/g;
(f) in the synthetic composition in a concentration of at least 1.0E+6 CFU/g;
(g) in the synthetic composition in a concentration of at least 1.0E+7 CFU/g;
(h) in the synthetic composition in a concentration of at least 1.0E+8 CFU/g;
(i) in the synthetic composition in a concentration of at least 1.0E+9 CFU/g;
(i) present in the synthetic composition in a concentration between 1.0E+5 CFU/g and 1.0E+10 CFU/g; or
(k) in the synthetic composition in a concentration between 1.0E+6 CFU/g and 1.0E+9 CFU/g.

67. The method of claim 66, wherein the trait of agronomic importance is selected from the group consisting of yield, root fresh weight, shoot fresh weight, biotic stress tolerance, drought tolerance, and combinations thereof.

68. The method of claim 67, wherein the trait of agronomic importance is biotic stress tolerance, and wherein biotic stress tolerance:
(a) is a growth environment comprising a fungal pathogen; or
(b) is shown by increased emergence, increased biomass, increased NDVI, decreased pathogen incidence, decreased necrosis, decreased chlorosis, decreased area of necrotic tissue, decreased area of chlorotic tissue, or increased yield.

69. (canceled)

70. (canceled)

71. The method of claim 68, wherein the pathogen is one or more pathogens of the genus Pythium, Rhizoctonia, Fusarium, Cercospora, Colletotrichum, Corynespora, Dreschslera, Diaporthe, or Macrophomina.

72.-81. (canceled)

82. The method of claim 49, further comprising a plant element.

83. The method of claim 82, wherein the plant element is:
(a) a monocot;
(b) a dicot; or
(c) is a whole plant, seedling, meristematic tissue, ground tissue, vascular tissue, dermal tissue, seed, leaf, root, shoot, stem, flower, fruit, stolon, bulb, tuber, corm, keikis, or bud.

84. The method of claim 83, wherein:
(a) the monocot is a cereal, and wherein the cereal is selected from the group consisting of wheat, rice, barley, buckwheat, rye, millet, oats, corn, sorghum, triticale, and spelt;
(b) the dicot is selected from the group consisting of cotton, tomato, lettuce, peppers, cucumber, endive, melon, potato, and squash; or
(c) the dicot is a legume, wherein the legume is soy, peas, or beans.

85.-90. (canceled)

91. The method of claim 49, wherein the synthetic composition further comprises:
(a) one or more endophytes; or
(b) one or more of a surfactant, a buffer, a tackifier, a microbial stabilizer, a fungicide, an anticomplex agent, an herbicide, a nematicide, a fertilizer, an insecticide, a plant growth regulator, a rodenticide, a desiccant, a nutrient, an excipient, a wetting agent, a salt, or a polymer.

92. (canceled)

93. (canceled)

94. The method of claim 49, wherein:
(a) the endophyte is dead; or
(b) the endophyte is produced in a low nutrient growth media.

95. (canceled)

96. The method of claim 91, wherein;
(a) the fertilizer is granular or liquid in form and comprises one or more of nitrogen, phosphorous, potassium, sulfur, magnesium, silica, iron, zinc, manganese, copper, boron, or fulvic acid; or
(b) the fungicide comprises one or more of fludioxonil, sedaxane, or mefenoxam.

97. The method of claim 49, wherein:
(a) the synthetic composition is heterologously disposed to a seed at a rate of 65-130 mL/100 kg seed;
(b) the one or more endophytes are heterologously disposed to a plant element prior to placing the treated plant element in or on a growth medium;
(c) the one or more endophytes are heterologously disposed to a plant element after placing the plant elements in or on a growth medium;
(d) the one or more endophytes are heterologously disposed to a plant element concurrently with placing the plant elements in or on a growth medium;
(e) the one or more endophytes are heterologously disposed to a plant element at least two times;
(f) the one or more endophytes are heterologously disposed to a plant element via a seed treatment or soil pre-treatment and one or more foliar applications;
(g) the one or more endophytes are heterologously disposed to a plant element via a seed treatment or soil pre-treatment and one or more floral applications;
(h) the one or more endophytes are heterologously disposed to a plant element via one or more seed treatments or soil pre-treatments, one or more foliar applications, and one or more floral applications;
(i) the one or more endophytes are heterologously disposed to a plant element via seed treatment, root wash, seedling soak, foliar application, floral application, soil inoculum, in-furrow application, sidedress application, soil pre-treatment, wound inoculation, drip tape irrigation, vector-mediation inoculation, injection, osmopriming, hydroponics, aquaponics, or aeroponics;
(i) the one or more endophytes are heterologously disposed to a plant element of a different plant variety from the variety of the plant element from which the one or more endophytes were obtained;
(k) the one or more endophytes are heterologously disposed to a plant element of the same plant variety as the variety of the plant element from which the one or more endophytes were obtained;
(l) the one or more endophytes are heterologously disposed to a plant element of a different plant species from the species of the plant element from which the one or more endophytes were obtained; or
(m) the one or more endophytes are heterologously disposed to a plant element of the same plant species as the species of the plant element from which the one or more endophytes were obtained.

98.-102. (canceled)


SEQUENCE LISTING (134 sequences)

SEQ ID NO: 1 - DNA, 20 bp, synthetic construct
  source 1..20: mol_type = other DNA
agagtttgatymtggctcag

SEQ ID NO: 2 - DNA, 19 bp, synthetic construct
  source 1..19: mol_type = other DNA
ggttaccttgttacgactt

SEQ ID NO: 3 - DNA, 19 bp, synthetic construct
  source 1..19: mol_type = other DNA
gtgycagcmgccgcggtaa

SEQ ID NO: 4 - DNA, 20 bp, synthetic construct
  misc_difference 8: note = n is a, c, g, or t
  source 1..20: mol_type = other DNA
  misc_difference 9: note = a or c or g; not t/u
  misc_difference 14: note = a or t/u
ggactacnvgggtwtctaat

SEQ ID NO: 5 - DNA, 1370 bp, Bacillus aryabhattai
  source 1..1370: mol_type = genomic DNA
agcttgcttctatgacgttagcggcggacgggtgagtaacacgtgggcaacctgcctgtaagactgggataacttcgggaaaccgaagctaataccggataggatcttctccttcatgggagatgattgaaagatggtttcggctatcacttacagatgggcccgcggtgcattagctagttggtgaggtaacggctcaccaaggcaacgatgcatagccgacctgagagggtgatcggccacactgggactgagacacggcccagactcctacgggaggcagcagtagggaatcttccgcaatggacgaaagtctgacggagcaacgccgcgtgagtgatgaaggctttcgggtcgtaaaactctgttgttagggaagaacaagtacgagagtaactgctcgtaccttgacggtacctaaccagaaagccacggctaactacgtgccagcagccgcggtaatacgtaggtggcaagcgttatccggaattattgggcgtaaagcgcgcgcaggcggtttcttaagtctgatgtgaaagcccacggctcaaccgtggagggtcattggaaactggggaacttgagtgcagaagagaaaagcggaattccacgtgtagcggtgaaatgcgtagagatgtggaggaacaccagtggcgaaggcggctttttggtctgtaactgacgctgaggcgcgaaagcgtggggagcaaacaggattagataccctggtagtccacgccgtaaacgatgagtgctaagtgttagagggtttccgccctttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaactctagagatagagcgttccccttcgggggacagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggatggtacaaagggctgcaagaccgcgaggtcaagccaatcccataaaaccattctcagttcggattgtaggctgcaactcgcctacatgaagctggaatcgctagtaatcgcggatcagcatgccgcggtgaatacgttcccgggccttgtacacaccgcccgtcacaccacgagagtttgtaacacccgaagtcgg

SEQ ID NO: 6 - DNA, 627 bp, Bacillus aryabhattai
  source 1..627: mol_type = genomic DNA
aaacattcccaaacatacaatatgaaagaactaaaaaccgctgtcggagcaattgttattgaaggacctgtctcctccaagaccttgcaacaacttgattttcatgagcaactaacggcctttcgtccacctgaacagcaaaaagaagcattgatcgagattgccaacttgcctgaaggtagaatcattattgctcgccatcatcatacaatcattggttatgtaacctttttatatcccgatcctctagaacgatggtcggaaggaaatatggagaatttaatcgaattaggagctatagaggtagccgctccatttcgcagctacgctgttggtaaaaacttgcttatcgtatctatgatggatgatgcaatggaagattatattgtcattacgacagaatactactggcattgggatttaaaaggaacaggactaaatgtgtgggattaccgcaaaattatggagaaaatgatgaataaaggcggactgacgtggtttgccacagatgatccggagattagctcccaccctgctaattgtttgatggtaagaatcgggagcagagtacctcaagaaagcatacaagaatttgatagcctgcgctttcgaaatcgctttatgtat

SEQ ID NO: 7 - DNA, 636 bp, Bacillus aryabhattai
  source 1..636: mol_type = genomic DNA
attgttgaagagatgatgaaaactgatctcgttaccttaacccccgaccatacgattgctgaagctatgaaacttttagatacacataagattagacatattcctatcgttaatgaccttcatcacgtagttggcatcatttctgatcgagacgtaagagatgcaagtccatctattttagataatacttataaatctgctcttctatccgaacccgtgcggatgattatgcaaaaagaagtcattacagctcaccctcttgattttgttgaagaaattgcaagtattttttatgaacatcaaatcggttgtattcccgtcacaaaaaacaagcggttagtaggcgttgttaccgaaagagatctattacatacgtttattcagcttaccggcaccaataagccaggttcacaaattgaaataaaagtagaaaataaggctggcgtgctcgctgaagttacatccctttttggttcaaagaaaacaaacatcttaagcgtactcgtctaccctgatgtagatgaacgatataaaatccttgtgcttcgcattcaagctattaatccattgattagtataaatacattaaaagaaaacggctatgaggtgttgtggcctaaattaccggagatgacg

SEQ ID NO: 8 - DNA, 1158 bp, Bacillus aryabhattai
  source 1..1158: mol_type = genomic DNA
actagtaatgctgtatttatttattcagatgatattctttcgtacaaattcagcaataatcatccgtttaaccagcttcgtgttcaactaacatatgaactgttacgggaatttggcgctttaggcaatcaagatattgtattaccgcggatggcatctgatgaagaacttcagcttgtccatgatactagctatattcaagcggtaaaattagcaggaagcggcgagctttctgttgaaaaagcagcagattttggtcttggaacagaagatacacctatgtttcccaacatgcatgaagctagtgcatttttagttgggggtacacttactgctgtagattatgtgatgactggaaaagctaagcatgctctaaatttaggaggaggattgcaccacggctttcgaggaaaagcttccggcttctgtatttataacgatagcgcggtagccatcaaatatatgcagcaaaagtacaacgcgcgtgttctctatgtcgatacagatgctcaccacggtgacggcgtgcaatggacattttatgacgacccttccgtttgtactctttccattcatgaaacgggacgctacttgtttccaggcacgggaaacgttaacgaacgcgggcacagtgaaggctatggttattcatttaatgtgcctgttgatgcttatacggaagatgagtcttggctcgacgtttataagacctctttaaaagaagtagctgctttttttaaaccagatgttatcttgacacaaaatggagccgattctcattattacgatccgctcactcatttatgcggcaccacgaaaatataccgtgagattcctaaaattgctcatgaaattgcccatgaatactgtcagggaagatggattgcggttggcggaggaggttatgatatttggcgggttgttcctcgggcttggtcattcatttggcttgaaatgattgaacgaaaggcagaaggcatgcttcctctgtcatggcttgaaaaatggacaaaagtatcgcctcaccccgtttgcacaaagtgggatgatgaacctaatatctatcagccaattccaagaaaaccagaaataacagaaaaaaacgctcaaactcttgagcgcgctctttatcctattcgttctcatacatctcattct

SEQ ID NO: 9 - DNA, 1671 bp, Bacillus aryabhattai
  source 1..1671: mol_type = genomic DNA
agtaatgctgatactaccgaaacaaaaaaaggcgccgacttactagtagaaagtttggcaagtcagaatgtgccttatatttttggcattccaggagccaaaattgatggcgtatttgatgttttgaaagaaaaaggaccagaagtaattgtttgccgccatgaacaaaatgctgcgtttatggcagctgctatgggaagactgacaggtacgccgggcgtttgccttgtcacatcaggtccaggtgccgctaacttagccactggccttgtcactgctacttctgaaggtgatccagtggtagccctagcaggagccgttccaagagcagatcgattaaaaaagacgcatcaatcaatggataatgaaggactatttaaatctatcgccaaattcagcgttgaggtagaagacgctgcgaacgttccagaagtgataacagacgcctttcgcgaagctgtttcttctccttcaggagcatcttttgtcagcttgccgcaggatgtaatggataacgaaacatcagcttccactctatccgttttaaaagctcctacgattggcgctgctaatttagacgctgttgcccaagctatacacactattcgcacagcaaaactacctgttttacttttaggtatgagagcaagtaaatctaaagcagtggaatcaatccgtcagcttatagcagaacatgaccttcctgtggtggaaacgttccaaggtgcgggagctgtttcgcgagagcttgaaactcacttttttgggcgagtaggtctttttcgcaatcagcctggagacgtgttattgaagcatgctgacgtcgtcttaacaatcggatatgacccagttgaataccctccttctctttggaactcagaaagtcaaagaagcattattcatttagatgatttacaagcacaagctgaccaccattatcagccggaaattgagttagtaggggatattccaagcacaattgacatattagcggataaactcccacatttaatacgttcaaaagaaagtgcttcttttttaaaaacgctaaaaagcaaacttgaacaacctgacgatcagtacgagttttctaatgacactcaccttcatcccctacaattaattgaagctctgcagaactttgttgaagatgatactaccatcacatgtgacgttggttcgcattacatatggatggctcgttattttcgttcctatgaacctcagcatttactattcagcaacggcatgcagacactaggtgttgctttgccttgggctatttcagctgcactcgttcgcccccataaaaaagttctgtccatgtctggagacggcgggtttttattttcagctatggagctcgagacagccgttcggttaaacttaccaattgttcacattatctggaacgatcaaacctacgatatggttggctttcaacagcagattaaatatggccgaaaatctgcagtagagtttggcagtgtagactttgttacgcttgctgaaagttttggtgccaaaggatttagagttaactcaccagaagaattttcggacactctagcaagcgcttttcgtgaacagggccccgttatcatcgatgttcctgtagattataaagataatgaaaaattaaatacacagctactgccagatcaaattaac

SEQ ID NO: 10 - DNA, 1590 bp, Bacillus aryabhattai
  source 1..1590: mol_type = genomic DNA
aaagtgaccgacacacttgttcaatgtttagaaaatgagggagttgaatatattttcgcaatacctggtaaagagacattggattttgttgattctttatccaactctaaacagattcaattcgtgaatgtgaggcatgagcaaggagctgcttttatggcagatgtatacggaaggttaacaaacaaagtaggggtatgcttatcaacattaggccctggagctactaatttattgacaggtatatctagcgcaaagctagaccattcccctgttgttgctttaacaggacaggctgctttgggaaggcagcataaagattcgcatcagtatatggacattatcaaaatggtggaaccggcaacgaagtggggtgttcagataaaagatgcacaaacgcttcctgagattattcgaaaagcatttaaaattgcacaaatggaaaagccaggacccgtagtagtagaattgccagaagacttagcaactcaaatcgttcccattaaacctattttacctacatctatacccgtaagcatacctatattggagtccattcaggctgccaatactcttattaagcagtataagaaaccttttgttattatcggtaacggcgctgttagacagggagccgtggatgagcttcaaccatttattgaacgccttcaatcaccggttacccatagttatatggctaaaggtgtattgtctaaagatcatccacaaaacttctttacgtttggatttaatgaaaaagatgaagtgctcgcaggcttaaaggaagctgatctacttattgtaattgggtttgattttgtcgaaaggttacccaaagaatggaacacggaaaaaacaccaattttacatatagatactttacccgcagaagtagatgaatattaccctatagaggtcgaattggttggagatataaagcaaactttacgagtattcaatcagcttgatattccttctaaaccatggatcccctctggtaatctccaggaacaaattaagcaatcttatcacattgaagagaaaaagatagatagcaatttgctgcctttgacaatcgagaatatcttgcatattattgacaagctctcttcagagaaaacaattgtgatttcagatgtgggagctcataaagtatctatagcccgaacttatcaacctaaaaaccctaacaggctcattatatcaaatggtctagcttccatgggtattgctctaccaggctcaattggagctaaactagcctgccctaatgatccagctatttgtattactggagatggaggagctttaatgaattttgctgaaatagagacggcaaagcgcttagggctttcattcatcattattgttttaaacgactctacattaaaaatagagcaggaaatgatgaacaaaaagtttggtaagagctatggagtaacttttacaaacccagattttgttcaactggcaaaaagctttggaattaaaggggaacaggcaaatagtttagttgaatttgaaaatataatgaaaagagccttacaaagtttagatgagattgtcctaattgacgttcttctaaaaagc

SEQ ID NO: 11 - DNA, 1044 bp, Bacillus aryabhattai
  source 1..1044: mol_type = genomic DNA
aaagcattaagatggcatggcgttaaagatgtaaggttagaacatattgcagaacctaccgtctctcaagggaaagtgaaaattaaagtagaatggtgtggaatttgcggaagtgatttgcatgagtacacggcaggacccatttttattcctacagaagaaccccatccattaagtaaagataaagcgccggttgtgctggggcatgaattttctggacaagttgtagaaatcggagaaggtattacgaatgtaaaagtcggagacagagtagtggtagaacctatctatgcatgcggagagtgtgaaaactgtaaacaaggaaagtataatctttgtaataaaatggggttttatggtcttgccggaggtggcggaggtttttctgagtatgcctccataccggtagaaatgattcataaaataccggaagtggtatcttacgagcaaggagctctcgtagagccttcagctgtggctcttcatgctgtgcgtcaaagcaaactaaaggtaggagataaagctgccgtattcgggacgggaccaatcggtttgttagttattgaagcgttaaaagcatcgggtgcatctgaaatttatgcgatcgaactatcggaacagcgcagacagaaagctgaagaacttggggcgattgtgattaacccgcaagaatacgaccccgtagcagaattacataagcgcacaggtggaggtgtggatgtttcctatgaagtgacgggtgttcccttagttttaactcaagccattaattctaccaaaagtggtggggagacaatgattgttagcatatttgaaaaagaagcgtccattcatccccaaacaatcgttttacaagagcgtacagtaacaggtattattggctaccgcgatgtatttccagcggttattagtttaatggcaaaaggctatttttctgctgataaactggtgacaaaacgtatcaagctagagaatgtaattgaagaaggttttgagacattgttaaaagaaaaagaacaggtgaaaattttggtgaaagctgaa

SEQ ID NO: 12 - DNA, 708 bp, Bacillus aryabhattai
  source 1..708: mol_type = genomic DNA
agcaataaagaaaaattaacgattcaccaaacttccactatgatcgctttattagacggagtatttgacggtgttgtcacatacaaggatttagctaagaaaggggatttcggcattggtacctttaatcaattggatggtgaaatgattgcctttgacggagaattttatcatattgctgatggtaaagccgcacctattagtgaagatgcaaaaacaccttttgccattatgacaaccttttatgaagatatctcttatcgtgtagaaaaggaaatgaatcgtgaacaactagaaaccttaatgaacgagcttattcctagcccgaatttattctatgcagttcgcattgacggcaagtttaaagaagtaaaaactcgcacggttgcaaagcaagaaaagccctatccttctatggtagatgcggctgctgatcagcctacatttacatcttcagatgtaaatggaacactagcaggtttttacacgccagaatattcttcaggcatcggtgttcccggctaccacctgcactttattgatgaatataaaaaagaaggggggcatgttttagatgtctccgttcaggatgttacaattcaaatctgcaaaaaaatgaaattaaatctcaatttatcggaaacaagtgaatttttaagtgctaatttagaagaccatgatgtagaaaaagaaattgccacggctgaa

SEQ ID NO: 13 - DNA, 1701 bp, Bacillus aryabhattai
  source 1..1701: mol_type = genomic DNA
caaacgaaagaacctgtaagcactggggcacaaatgaatggagctgacatgttgattgaatctctaagaagagaaaatgttgacgtcatcttcggttaccccgggggagccgttttaccgatttacgataagctatatggttcaggaatgtttcacgtacttgctcgtcacgaacaaggtggaattcacgcagcagaagggtacgcaagaatttcaggcaaacccggtgtagttatcgcaacaagtggtcctggcgcaacaaatatcgtaacaggtttggcagatgcgatgattgactcacttcctttagttgtttttactggtcaagtagcttcaagtgtaattggttctgacgcgttccaagaagcagatgtattagggatcacaatgccaattacaaaacatagctatcaagtacgcaatatcagcgatatgccgagaatcatcaaagaggcatttcatattgcgacaacaggaagacctggtccagtcttaatcgacattccaaaagatatggcacagggtgatggaacatttaactacgaaggagaaattgatttacctggctatcagccaacttattttccaaatcagctgcaggttaaaaaattaacagacgcagtagcagctgcaaaacaacctgttatcctagcaggtgcgggagtgttattgagtaaaggttcagaagaattaaaaaattatgccgagcgtcaaaatattccggttattaatacgcttttagggttgggctgttttcctgcagatcatccgcttcatttaggaatggctggcatgcacggaacgtacacagcaaacatggcaatttatgagagtgatcttctcattagtatcggtgcacgctttgatgaccgagtaacaggtaacttaaagcattttgcaccaaatgcaaaaattgctcacattgacatcgacccagcggaaattggaaagaatgttccaaccaagattccaattgttggagatgcaaaagaagtattgaagcaattgcttctgcaagaaggagaagttggagattatacgaaatggcatgagaaattagcggcgtggaagcaagaatatccattatggtatcagaactcagcggacgtgttaaaaccacaaaaagtagttgagttgcttcacaagtatacaaatggagatgccgttgttacaacggacgttgggcaacatcaaatgtgggtagctcaatactacacatttaataatccagatcgatgggtaacttcaggtggtcttggcactatgggctttggattaccttctgcaatcggcgctcagctggcaaataaagataaaacagttgtagcagtcgttggtgacggtggatttcaaatgacgcttcaggaattaggggttattagagaattaaatttacctgtaaaagtggttgtactcaacaatcagtcgcttggtatggttcgtcagtggcaggaaatcttctatgaagaacgctactcacactctttaattccgaatcagcctaatttaatgaagcttgcagaggcttatgacattaaaggtttacaagcttctactgaagaagaagcgcatgcggctctgaaagaagcaatggaaacagatggacctgttctactaaatttctttgtcgcacctaaagaaaacgtatatccaatggttgcacctggaaaaggattacatgaaatggtaggggtgaaacca

SEQ ID NO: 14 - DNA, 507 bp, Bacillus aryabhattai
  source 1..507: mol_type = genomic DNA
aagcgaatcattacagcaactgtattaaaccgtgcgggcgttttaaatcgcgtaacggggttatttacaaaaagaaatttcaatattgagagtatttcggtagggcacactgaatctgaggatgtatcacgcatgacgtttgttgtgaacgtagagcatgataacgctgctgaacaagtgatcaagcagttaaacaaacagatcgacgtgttgaaagttcaagatattacggatcaagctgtggtgtcaagagagcttgctttaatgaaagtagcctctacaccagcgagccgtaacgaactttatgcagtaattgaacctttccgtgcaaccatcattgatgtaagtcgcgacagcctaattattcaagtaacaggtgaatccagcaagattgaagcgatgattgatctacttcagccttatggattaaaagaagtaacgcgcacgggaacagtagctgttccgcgcggtacgcaaaaagcggcagccaaactttcatcaatcatt

SEQ ID NO: 15 - DNA, 576 bp, Bacillus aryabhattai
  source 1..576: mol_type = genomic DNA
aaaatagctgtaggaatcacaggagccacgggagctattttaggtattcgaattttagagcttttgaagaaggcttctgttgaaacgcatttagtcatgtcgccttgggctcatgccacaattaagctggaaacaccctatagcgttcagcaagtagaagcgctagcagattattgttattcatatcaagaccaagcagcaaaaatttcgagcggttcttttcgcatagatggaatgattgttagtccttgcagcatgaaaacattagcatctattcgaatgggactagcagataacttaatagcgagaacagcagatgtgatgctaaaagaaagaaagccactggtgctacttcctcgagaaacgcctttgaatacgattcatttagaaaacatgctggatctttcaaaaatgggagctatcctcgtgcctcctatgccggcattttataacaagcctgagacgattgaggatattgtcacccatattgctgttcgaactttagatcaattaggaattgagcttcctgaggcaaaaagatggaatggaattaaacatttatcacaaggaggaaaa

SEQ ID NO: 16 - DNA, 1416 bp, Bacillus aryabhattai
  source 1..1416: mol_type = genomic DNA
gcttataaagactttagagatttccttaatacgctacataaagaagggcagctactgacggttacagatgaagtgcagcctgatccagatttaggttcagcaggtcaagccatcagtaatttaggagaccaaacgccgggattattatttaccaatatttatgggtacaacaatgcaaaggtagctctaaacgtaatgggttcctggtcaaatcacgcgttaatgatgggactgcctaaatcaactcctgtaaaagaacagttctttgagtttgctcggagatatgaaaaatttcctgtcaaagtgaaaagggaagaaacagcgccatttcatgagtgtgaaattaaagatgatatcaacttattcgaccttttgccattgtttcgcttaaatcaaggagacggcggctattatctagataaagcgtgcgtcatttctcgtgatcagcatgataaggagaatttcggcaaacaaaacgtgggtatatatcgtatgcaggtcaaagggaaagaccgtctaggcattcagcccgtgccacagcatgatattgccattcacttgaaacaagccgaagaaaaaggtgaaaacctccctgtatcaattgctttaggatgtgaacctgcgattgtcacagcagccgctacgccgcttcattacgatcaatcagaatatgaaatggcaggagctattcaaggtgagccgtacagaattgtgaagtctcagctttctgatttagatgtaccgtggggagcagaagtgattttagagggagagatcttagctggtgaacgcgagtatgaaggtccttttggtgaatttacaggtcattactccggtggcagaagcatgcctgttatcaaaatcaatcgcgtataccatcgtaaagatcctatttttgaaagtttatatatcggtatgccttggacagaaacagattatttaattggaatcaatacaagcgttcctttatatcagcagctaaaagaagcatatcctgaagagattgaagctgtgaacgcgatgtatactcatggtctcgttgctatcgtttcaacaaaaagccgatacggaggatttgctaaggctgttggtatgagggcattgactacgccccatggattaggatattgcaagctagttattttagtagatgaagacgtggatccgtttaatttaccgcaagtcatgtgggcgctatcaacaaaaatgcacccaaaacatgatgttataacagtgcctaatctttcagttctgccacttgatccaggatctgagccggctggtattacagataaaatgattttggatgctacaacaccagttgcaccggaaacaagaggccactattctcagcctttagatacaccacttgaaactgaaaaatgggaaaaaatcttaacgaatatgatgcaaaaa

SEQ ID NO: 17 - DNA, 1488 bp, Bacillus aryabhattai
  source 1..1488: mol_type = genomic DNA
caaaatcttggagcgaaaaaagttgaaatgagtccgaaagtagtggagtttttaaaaggagttaaacagctatacattaacggcgaatgggtagactctgtttcaggaaaaacgtttgaaacagtaaatccagcaacgggtgaaaagttggcagatgtagctgaagcaaatccagaggatattgatcgtgccgtcagagcggctagagaagcctttgatcatggaccttggacaaagatgtcagcagctgaaagaagtcgacttatttataaattagctgatttaatggaaacacataaattggagcttgcgcagcttgaaacgcttgataacggaaaaccaattcgtgaaacatcaaatgcagacattccattagcaattgaacatttccgctactttgctgggtggtcaactaaaatggttggacaaactattcctgtgcaaggagcgtattttaactatacaagatatgaaccagtaggggttgttggccaaattattccgtggaactttccacttttaatggccgcttggaagctcggagccgctttggcaactggatgtacaattgtgctcaagccagcagagcaaacgccgctttctgcactttacttagctcgactagccgatgaagcagggttcccaaaaggcgtgctaaacattgtgccaggctacgggaaaacagcaggtgaaccgctcgttcatcatgatcttgtcgataaaattgcttttacaggatctactgctgtcggaaaagcaattatgaagcaagctagcgactccttaaagcgtgtgacgttagagcttggaggtaaatcaccaaacattatcctcccggatgcagacttaacaaaagcagtgcctggtgcgctttcaggtattatgtttaatcaaggacaagtatgttgtgctggaagtcgtctgtatgtccaaaaaagcttatatgataatgtcgtagctgatttagtttctcaaacgaaatctattaaacaaggaaatggattagcagatgagacgacaatgggtccgcttgtatctgcgcagcagcaaaaacgagtgaaaacctatattgataaaggaattgaagaaggagcggaagtactcgcaggaggaaacattccatttgatcaaggctatttcgtagaaccgactatctttgcagatgtcgatcattcgatgacgattgctcgtgaagaaatcttcggtccggttgtagctgctatgccgtttgacgatcttgatgatttgattgccaaagcaaatgatacagcatatggtctagcagcgggcgtctggacacaggatttaaagaaagcacactatattgctcatggtattaaagcgggtacagtttgggtgaactgttacaacgtatttgatgcagcaagcccgtttggaggttataaacagtcgggaattggtcgtgaaatgggaagctatgcgcttgataactacactgaagtgaaaagcgtatggatcaatatggaa

SEQ ID NO: 18 - DNA, 1167 bp, Bacillus aryabhattai
  source 1..1167: mol_type = genomic DNA
tctacttattcttttgatcaattaattaaccgtacaggtacgcagtccgtaaagtgggacaaaactgatcttgtttttggagcaaaagatgttctgcctttttgggtagctgatatggatttccaagcgccgcctaatgttcttcaaaccattaatgagcgtgtcaaacacggtattttcggctacacttcccctactgaatctacggcaaatgctttatcatcttgggtagaaaaaaagcatcagtggaccattgatcatgaatggattacatacagtccaggagttgtatttgccctgagtatggctatccaagcatttacggaacccggagattcaattttaattcagcctccagtctatactccattttttaacatgataaaggtaaatgatcgagaagttgttgaaaatccgttaaaacttgcaggcgataaatacgaaattgattttgcagatttagagcaaaaatttgcagaaggcataaaattaatgattctttgcagccctcataatccagtaggacgcgtgtggacaaaagaagaactgaagaagcttgccgatctttgtgaaaaatacaatgtattaattgtttcagatgaaatccattctgatctaatttaccagccttataaacacattcccattagcatagtgagtgaagatgcagccaatcgaaccattacgtgtatggcccctagcaaaacatttaatataccgggacttcaagcttcagctattattattccaaacgagaaactgcgcacacagtatgttcaacaacagcaaaaacaagggttttctaatttaaacacattcggaataattgggttagaggcggcttacagtgaaggtgaggagtggctagaagcactgcttgtatatctgaagaaaaatgtagatctcgtgacgcgttttataagtgaaaacttgcctgaactgcgagtggttcagcccgaaggaacttatttactatggattgactgcaacaagctgcatttatcagaagaagaattacatgaacgcttattagaaaaagggaaaattgccgtagaaaaaggcgaaaaatacagttccacatatggaaaaggttttatccgcttgaacattggctgttcacatgaacagcttcaagaaggattaaatcgcttaaaagtagctttaacg

SEQ ID NO: 19 - DNA, 1563 bp, Bacillus aryabhattai
  source 1..1563: mol_type = genomic DNA
aagagagagaaagggttgtttgagcgtatgtctacaatagatttatcaaaattcgagaaaaaaatgattattcgtaacatcaaaccggacgatatagatgaaattttagcacttcaaaagctatcattcccgggaatggatccgtggaaaaaagaacaacttgaaagtcacctggccatttttccagaaggacaaatatgtgcagagtacgacggggaaattatcggatcttgttcaagcttgattgtgaactttgatgagtacgatgatcagcatacgtgggacgatattacagacgaaggatacattacaaatcataacccggaaggctacaatctgtatggaattgaagtaatggttcatccaaagtatcgcggcatgaaaataggacgccgcttatatgaagcgcgtaaagatttagcacgtgaattgaacctgaaaagtatcatcattggaggaagaattcctaattaccataagcattcagctgaaatgtcgcctagagaatatgtagagcaagttattcatcataaaatttatgatccagtgctatcttttcagctaatgaacggatttacgcttatgcgtataaaccctggttatcttcctgatgatctagcttctaataagtatgcaacgttgatggaatggaacaacgtagattacgttcgccgttcaaaacgccattttaaaacatcattcccagtgcgtatttgtgttgttcagtatatgatgaaacagatcaattcatttgaagagtttgcaacacaggtagaatattatacagatgttgcttatgatgcaaaagccgacttctgcgtattcccggaaattttcacaactcagctaatgtctttcttgaatgaaaagatcccaagcaaagcggttcagcgcgtagcagaatatacggaagagtacattgagctattcactaacttagcggttaaatataacgtaaatattattggaggatcccactttgtcgaagaagataatggagatatctttaatattgcctatctattccgccgagatggaacaattgaaaagcagtacaaagtgcatattacacctaatgaaagaaaatggtggggaattagcccaggagataaagttgaagtgtttgatacagattgcggtaaaattgcgatccaaatttgctatgacatcgaatttccggagcttgcccgtattgctaccgataaaggtgcaaatattatttttacgccattctgtacggaagaccgtcaaggttatctgcgcgtaagatactgctctcaggcgcgcgcgatcgaaaaccaaatttatacggttatcgcaggtacagttggaaacctgcctgaaacagaaaacatggatattcagtatgctcagtctgctatttttgcaccgtctgactttgaatttgcacgagacggcattgtaggagaatgtaatcctaatattgaaatggtagttattggagacgtagatttagaaattcttcgccgccagcgtcaatcaggaacggttcgtcaattaaaagaccgacgccacgacgtttacaatattaaatataaaaaa

SEQ ID NO: 20 - DNA, 459 bp, Bacillus aryabhattai
  source 1..459: mol_type = genomic DNA
attaaaataaaaagagaccacttaatgaacaatgaagttattgaactggtaagttctcaccttcaaggaatgaacttacattcaccccctgaaagtattcatgctctaaatattgaaaacttaaagcaacgtgatattacgttttggagcgtgtgggaagacagtcagctagcagggtgcggggctttaaaagagttagatgagcatcacggagaaattaaatcgatgcgaacagctaccgcccatttgcggaaaggaatcgctaaagagctgctgcagtacataataaacgaagcacaaaaacgagggtaccgacgcttaagcttagaaacaggatccgcgtctgcttttgaaccggctagaaagctgtacgccagttttggattttacgagtgcgaaccgttttctacctacaaagaagatccatacagtgtatttatgacaaagaaattgaatgat

SEQ ID NO: 21 - DNA, 951 bp, Bacillus aryabhattai
  source 1..951: mol_type = genomic DNA
agcataggtcaaatttttctcattctcgcgcctatcttttttgtcattttgcttggttatttagcagggcactttaaaaaatttgataagcaaacgtctaaggggctcaacacattagtaacgaagtttgcgcttcctgcacacctatttgtaggtgtaacgacaacgccaagaaaaacactcatagacgaatggccgtttttagtagcactgattttaggaatcgtcgggttttatctcattatattgctcgtcgtgaagtatgcagggaagcatacgttaactgcctcttctatgtttgccctaaactcagctcagccaacgtttgcttttatgggcatcccagtactcggaggattgttcggtgcaggcgcagtagcgattccaattgccataacaggaatcgtggtgaatgcgattttagatccgctggcaacgattttaggaaccgttggacaacgggggcgaaaagaaggggaagagaaaacaagtttatttaaagtaacggttcattccattttgcatggtttatctgagcctttggcatgtgtaccgttaattggggtgattttaacgctttgcggctttcagtcacctaaacttttacaggatatgcttgatcaaatcggaagtatcacatcgggagcggctttgtttgcggtaggagtaacgattggaattggaaaaattaacttcagtaaaggtgcgcttgggatcgctgtcttaaaagtggcggttcaacccatcgttatgctcggaattgcagtatggatagggctgtcttcagatgaagtgacaaaagctattttactggtagcttttcctgggtctgctgtagctgcgatgattgctttgcggtttgaaagtttggaaaacgaaaccgcatctgcttttgtcattagtgctattttatcgttgattacgctgccgcttcttatttcttggcttatg

SEQ ID NO: 22 - DNA, 951 bp, Bacillus aryabhattai
  source 1..951: mol_type = genomic DNA
gaaattagtcaaatctttattattttaaccccaatcttttttgttattttgctaggttatttagcagggtattttaaaaagtttgatgcaacaacatctaaagggttaaatacgcttgtaacaaaatttgcgttacctgcacatctattcgttggtattacaacaacatcaaaacaaacgcttattgaaaaatggccgtttttactagcattagttcttggtattattggattttatgttcttttcttacttgttgcaaaatatgtatttaaatactctttaacaggcgcgtctatgttttctttaaactctacgcagccaacgtttgcgtttatgggtattccagtattaggtagcttgtttggcgcggatgtagtagcaattccaatcgcaattactggtatcgtagtaaatgcgatgcttgatcctttagcaactattattggtacggttggacagcgtgaaagaagtgaaggcgattcagaagaaagcttatttaaagtaacgattaaatcaatcttacacggtctctctgagccgttagcctgtgttccgttaatcgcagtaatattagtattatgcggcttccaagctccagatttactatcaagcagcttagatcaaatcggttcaattacatctggtgcggctctattcgcagttggggtaacaattggtatccgtaaaattcaatttagcccagcggcatttggtattgcgattttaaaagtggctattcagccgcttgtaatgttaggaattgcaacagcaatgggtctatcatcagctgacatcgtaaaattagtattattagtagcgttcccaggttcagctgtagcagcgatgatttcagttcgctttgacagcctagctggtgaaacagcttcagcattcgtacttagtgccattatgtcacttgttacattaccgctgttaatttcttggttaatg

SEQ ID NO: 23 - DNA, 972 bp, Bacillus aryabhattai
  source 1..972: mol_type = genomic DNA
cctatagatgaaagtagtgttgttatgacgggagagaaacccaaacttattgtcattattggaccaactgcggttggtaaaacaaaattgagcattgaacttgccaaacagttagacggagaaattatttcaggtgattccatgcaaatttataaagggatggatatcggaacagcaaaggtgtcagaagaagagatgcagggtattccccattacctgatcgatattaaggagccagatgaatcgttttctgtggcagaatttcaagagattgtaagaaaacaaatcgatgatattcatgctagaggaaaaatgccgatgattgttggagggacgggtctttatattcaatccgttatttatgattatcagttcacagaaactcctagtgatgaaaaagttcgtgaacgaatggaagtatttattaaagctcatggcattgatcccctgtatgaaaagctaaaagaaattgacccagacagtgcagctgctatccataaaaataaccatcgccgtgtaatcagagcgcttgaagtttatgaagtaaccggcaaaaaattcagtgactatacaaaagaacagtcgcaagagttattatatgacgtaagcattattggacttacaatggatagagacgtattatatgagagaattaatcaccgagtcgatatgatgatgcaggctggactgcttgatgaagtcaaatcattgtacgaaagtggtctgcatgatgtacaatcaattcaagctattggctataaagagctgtatgcttattttgaaggacgctgtacgctcgaagaagccgttgaacagttgaaacaaaattcacgcagatatgcaaagcgtcagttaacgtggtttcgaaataaaatgcctgtaaaatggatagagatggaagttgacaaatttcccgaaaagtttgctgaaatttttacatatattgcaggaaagctgaagaaagaagcgaat

SEQ ID NO: 24 - DNA, 1524 bp, Bacillus aryabhattai
  source 1..1524: mol_type = genomic DNA
aacgaaaaacaacgtttagaaagcaaccaagttaatccagcggacaaaaaatccgaaaaggattacagtaaatattttcaaacagtatacatgccaccttctttaaaagatgcgaaaaaacgaggaaaagaagaagtttcctatcacaatgattttgcaatcgatgaacagtttcatggaatgggacagggacgtaagttctatatccgtacgtacggctgtcaaatgaatgagcacgatacagaagtaatggctggtatttttctaggccttggctacgagccaactgactctgttgaagatgctcatgttattttattgaatacgtgtgcgatccgtgaaaatgcggaaaataaagtgtttggtgaattaggacacttgaaggccttgaagcgcgagcgccctgagctattaatcggcgtctgcggatgtatgtctcaagaagagtcagttgtaaataaaattttacaaaaacatcagcacgtggatatgatttttggtacacacaacattcaccgtttgcctcacattttaaacgaagcatacttagcaaaagaaatggtgattgaagtatggtctaaagaaggggacgttattgaaaaccttccaagagcacgtaaaggcagcgtcaaagcatgggttaacatcatgtacggctgtgacaagttctgtacgtactgtatcgttccgtatacgcgaggaaaagaacgaagccgccgtccagaagacattatccaagaagtacgtcatttagctgctcaaggctacaaagaaattacgcttttaggccaaaatgttaatgcatatggaaaagattttgaagacataacgtacggcctaggagacttaatggacgaaatccgtaaaattgacgttgctcgtatccgttttacaacaagtcatcctcgcgactttgatgaccgtttaattgaagttcttgctaaaggcggaaatcttgtcgaccatattcacttgcctgtgcaatcaggaagcagcgatatcttaaaaattatggcacgaaaatattctcgtgaacattatttagagcttgttcgtaaaattaaagaagccattccaacagcatcattaacaactgatattattgttggtttcccaaatgaaacggatgagcagtttgaagaaacaatgtcgctatatcgtgaggttgaattcgatagtgcttatacatttatttattcacctcgtgaaggtacaccagctgctaagatgcaagacaacgtaccaatggaagtgaaaaaagagcgtcttcagcgcttaaatgctctcgtaaatgaaatttctgcaaagaaattgaaggaatatgaaggtaaagttgtcgaagtattagtagaaggtgaaagtaaaaataatccggaagtattagcaggctacacggaaaaaagtaagcttgtaaacttcaaagcacctaagtcagtgattggtcaacttgtaaaagtaaaagttacaaaagccaaaacatggacattaaacggggaaatggtagaagaaacagcagaggtgaag

SEQ ID NO: 25 - DNA, 570 bp, Bacillus aryabhattai
  source 1..570: mol_type = genomic DNA
aaaagactagctgtattttgtggctcaagtaaaggggcatctgatctttatattgaaggagcaaaagctctaggaagagagcttgctaaacgcaatatcgctcttgtatacggaggtgtaagtgtcggtgtaatgggtgctgttgctgatgccgttttagaagaaggtggacatgtaattggcgttatgccgagctttcttgaggaaagagaaatttctcataaaaacttatcggaacttattgttgttgaatctatgcatgaaaggaaagccaagatggcagaactagtggatggatttattgcattaccaggtggtccaggcacattagaggaattctttgaaattttcacatgggctcagttaggtttacatcagaaaccatgtggacttttaaacattaataactattacacaccacttgtatcattatttaatcatatgacagaagaacagtttttacaggaaaaatatcgttctatggctcttgtagatacagaaccacatgggttacttaatcaatttaatacataccaaccaccttcagtaaaaacatatattacaaaagct

SEQ ID NO: 26 - DNA, 573 bp, Bacillus aryabhattai
  source 1..573: mol_type = genomic DNA
aaatcaatttgtgtgtttgcaggatcaagcgctggaataaaaacagaatataaagaggccgccgtctcgctaggacgctacatggcccaaaaaaactaccggctgatttacggaggctcgcgaatgggcctgatgggagaagtcgcaaatgaaatgcttcagcatggaggcgaagtcatcggcattatgccccgcggtttgtttagcggtgaaattgttcataccgcgctgacagaactcatcgaagtagagagcatgcatgagcgaaaggcaactatgcacgagttagctgatggttatattgcgcttcctggaggtttcgggacatttgaagagttgtttgaggctctttgctgggctcaaattggtatacataaaaaaccagtagggttattaaatgtgaacggctactacaacccgcttatgcaaatggttcagcatgccgtagatgaagggttttctacggattctgctattcgtctcatcaacatttctgatactcccgagcagcttatctcttcaatggacacgtacacgtccccttcagaggaccaaaaatggaacactgaacgc

SEQ ID NO: 27 - DNA, 576 bp, Bacillus aryabhattai
  source 1..576: mol_type = genomic DNA
aaaagaatagctgtattttgtggatcaagtaatggagcatccgaactttatattcaaggtgcaaaagctctgggaaaagagcttgctaaacgtaatatttcgctcgtatatggtggtgcaagtgtcggagtaatgggtgctgtggctgatgccgtgttggaagctggcggaaatgtcattggcgtcatgcctacttttcttgaagaaagagaaatttctcataaaaacctgtcagagcttatcgttgttgaatctatgcacgagagaaaagccaagatggctgaacttgtagatggattcattacattaccaggtggtccaggtacattagaggaattctttgagatttttacatgggctcagttagggttacatcagaagccttgcggattattgaatattaatgattattacacaccacttatttctttatttaatcacatgactgaagaacaatttctacaggaaaaatatcgttctatggctcttgtagatacagaaccacaaggtttattagatcaatttaatacatatgaacctccttcagttaaaacatatatcacaaaagctcaaagc

SEQ ID NO: 28 - DNA, 1434 bp, Bacillus aryabhattai
  source 1..1434: mol_type = genomic DNA
acaaagatgccaaaagattttttatggggcggcgcattagcagctcatcaatttgaaggtggatggaaccaaggtggaaaaggaccgagcgtagtagacgttatgactgcaggtgcgcacggtgtagcgcgacaaatcactgaaacagtggaagaagataaattctatccaaatcacgaagcaattgatttttaccaccgctacaaagaagatatcgcactgtttgcagaaatgggcttaaaatgtttacgaacgtctattggctggagtcgtatttttcctaagggcgatgaagctgagccaaacgaagaaggactaaaattctacgacgatgtatttgatgaactgttaaaacacggcattgagccggttattacgctgtctcactttgaaatgcccctgcatttggctcgagaatacggcggatttagaagccgaaaaacggtagattgctttgtgaagttcgctgaagtttgtttcgaccgctacaaagacaaagtcacgtactggatgacgtttaatgaaatcaacaacaaaatggacgtacacaaccctctattcttatggacgaactccggcgtttctgtaaaaccgggtgaaaacgcaaaagaagtgatgtatcaagcaggacatcatgaactgctggcaagtgctcttgccgtgtcaaaaggaaaagaaattaatccgaatttccaaatcggagcgatggtgtcacatgtaccaatctatccgtattcttcaaaccctgaagacgtgatgttagcagaagaaacaatgagacagcgctacttcttccctgacgttcaagttcgcggctactacccaagctatgcgttaaaagagttcgaacgagaaggctatcgtattccgtttgaagaaggcgacgaagaaattttacgaaacggaaaggtggattatttaggcttcagctattacatgtcaacaacggttaaaagcgacgttgaagctgataacacaggcgatatcgtaaacggaggattgccaaacggcgtagcaaatccttatatcaaatcaagcgattggggctggtcgattgatccaacgggcttgcgctatacgttaaaccgcttctacgaccgctatcaaattccgctgttcatcgtagaaaacggatttggcgctatcgatacagttgaagaagacggatccattcatgacgcatcacgaattgaatatttaaaatcgcacatcgaagcgttagaaaaagctgtgacgtacgatggtgttgatttaatgggctacacgccgtggggaattattgatattgtttcctttacaacaggcgaaatgaaaaagcgatacggcatgatttatgtcgaccgtgacaacgaaggaaacggatcaatgaagcgctacaaaaaagattcctttaactggtataaacatgtaattgaaacaaatggtgaagaacta

SEQ ID NO: 29 - DNA, 1488 bp, Bacillus aryabhattai
  source 1..1488: mol_type = genomic DNA
ttaacaaatgatactgtatttaaaaagggatttttatggggaggagcaacagctgctaaccaaattgaaggagcttatgacgttgatggaaaagggctgtctactgcagatatgatccgatttgtcccaaaagacgaacgaactggttattcgatggatgtaaaagcagattatattgaaggtgtgcttaacaacaaagtaaacgatcgttttccaaagcgtacggctatcgacttttatcatcgctataaagaagatatcgcattattcgcggaaatggggtttaaaacgcttcgcatttcgatcaactgggcgagaattttccctaatggagatgatgtgaatccaaacgaagcagggttgcaattttacgatagcctatttgatgaaatgttaaagtacggaattgagccgttagtgacgctttcgcattatgaaacgccgctggagcttacgcgtaaatacaacggctggtacagccgcaagctaattggcttttatacaagctacgcagaaacggtatttacacgctataaaaacaaagtgaaatactggctgacgtttaatgaaatcaatgtgattacccatagtccttatacaggcggcggtattttagttgaaaacgttccaaatcagtcagcggaacaaattgcgtatcaaggagcgcatcatcagtttgttgcgagtgcacttgccactaagctagctcacgagattattcctgatgtaaaagtagggtgtatgcttgcgcgtatgacaacgtatcctgtgacgaatcatccggacgatattttactagcacagcataaaaatgacttgaacttattttttacagatgttcaagtgcgcggagaatatccaaaggtaatggagcgttactttaaagagaacaacattcaaatcgtaaaagaagtaggggacgatgaaattatcaaacaatatcccgttgactttatttcatttagctactatatgtctctttctgtcacaactcaaagtgaagaagaaaatgtcctaggaaatatgattgcgggaagtgtaaaaaatcctcaccttcaaacgtccgattggggatggcaaatcgatccgaaaggacttcgctatacgttacaagattttcataaccgttacggcaaaccgctgtttattgttgaaaatgggttaggagctcatgacaaagtagaagaagacggctcgattcatgatccgtatcgtgtagattacttaagacagcatattgaacaaatgaaagaagccgtcaaagatggtgtagacttaatgggctatacagcgtggggacctattgatttaatcagtgtgtcaacaaacgaaatgtcaaaacgatacggctttatttacgtagatcaagacgatgaaggaaatggtacgcttgaacgatctaaaaaagattctttttactggtataaacaagtgattcagacaaatggagaagatttatcttcatctccaaaagtcaaagtttctcag

SEQ ID NO: 30 - DNA, 615 bp, Bacillus aryabhattai
  source 1..615: mol_type = genomic DNA
aatcaaaattcaccaaaatatgttgctttaacgtttgatgatggaccctcagcctacaccgttgctattttaaaaattctaaaaagatataatgtacgtgcgacattttttgtcgtaggtgcagaagcggaaaaatttccaagattaatccgacgtattcatagagaaaagcacgtaattggaaatcatacgtggagtcatcctgatattacaactctttctaaacgcgaattatggaaagaaatcacttctacaaatgctcaaatagagaaaataataggttgttctccaaagctgtttcgtccaccttacagttcgataaatgatacagctcttgctgatattaaagaattgggaatgacgtctgttctatggaatgtagatagtcaggattggcgtgaagaagatccattggccatatgcaaacatacgattaaaaatctacaagaaaagaatcttattgtcatgcacgatggggatcgatacggcagcggagcgcgagaccaaattgttacttcattgccgaaacttattaagtatttaataaataacgactatcaatttgtaacggttcctgaatttcaccgagtggcattcaaaattgaaggatggtct

SEQ ID NO: 31 - DNA, 1182 bp, Bacillus aryabhattai
  source 1..1182: mol_type = genomic DNA
aaacattcttacccttcatataatcgcccgcctgctctaagacagctctacaagtatttatggattttttttctcttgctctttctcataactttaattattcctactatgattgtgttttcaacaggtagtggatctcttgcgatgaataaatccctaacttcttcaatttcacaaaagtcttctgcagtcactgaagacaacaaaatttcttctgtttctgttaataaacatgtttcacctaaagaatctcctttgtctataggatgggtaacaggaaataataataactcgagtacacaagatttatctgcttatcataatcttaaagttgtttctccagcattagtgtctattaacaatcagtataacttacaagtgaactctaacccctctttaataaacactatgcatacccaagataaaaaaatatgggcacgttttatcattaataatgatacaaaatctaatgttcatacatttctttctaatcctagtaaaacccaagaaataatcaagaaaatttaccaagtttcccttaataaccaatgggatggtatcaatttagatattgaaaacgtgcgtagtcaagatagggatcttttttcacaatttgtaaaaagtttatctaatctcctaaatcaatcaaagattgttctttctattgacctacctcctgattctaaaggaagtaataataaatatggaccatttgatcataaaataatagggaattattgcgattacattatttttatgggatatgatcaacattggtcaacagaccctgtacccggacctgtaacatcactctcatggctaaaggaaaatgtacaagagtttacgaaaactggtatacctcctcaaaaaatgatattaggccttcctgcttatacaagggtttggcaacaagacaatagtgggaagattgtaaaaaaacctgccacttctgttcaatatgttgaagatttagttactcaaaatcatcgaaatttaaattggaattccactttgggggaatattacacttcttacactgcaaataacagagtttatcaagtatggctccctactgttacatcctttaaagaatatttagatttaattccgcagtatcatctagctggatctgctatatggagcctagatcttatgagcgcatcatattggaatcaaatattt

SEQ ID NO: 32 - DNA, 324 bp, Bacillus aryabhattai
  source 1..324: mol_type = genomic DNA
gaaagcattcaagaatatatttttaaattaatcttacacggtggaaacggcagaagttcagccatggaagcgatagcagctgccaaaagcggaaatttttcagaagcacgtgaaaagcttacacaagctgcagaagaattaaatgctgctcatcatatccaaacgtcgttaattcaaggtgaaattagaggggaaagcacagaaatttctctgctcatgattcatgctcaagatcatttaatgaatgcgatgacggtaaaagaaatggctactgaatttgtagatttatatgaagcaatcaaaatttctggggggattacacta

SEQ ID NO: 33 - DNA, 300 bp, Bacillus aryabhattai
  source 1..300: mol_type = genomic DNA
aatattctattatgttgtgcagctggaatgtctacaagtttactagtaacaaaaatggaaaaaagcgctcaggatcaaagcacggagtataaaatttgggcggtccctggtgactctgttcgcaaccatattgatcaagcggatgttcttttattaggacctcaagtgcgctacttgctgccgcagcttaaaaagctaggtgaagaaaaaggagtaccagttgacgttattaatcctgttcactacgggacgtgcaatggagaacaagtgttaaaatttgcggttcaactagtttcaaaa

SEQ ID NO: 34 - DNA, 300 bp, Bacillus aryabhattai
  source 1..300: mol_type = genomic DNA
aatattttactatgctgcgcagcagggatgtctacaagtttgttagtgactaaaatggaaaaaagcgcaaaagaaaaagagaaggcgtgcaaaatctgggcagtaccaggagacgcggttagaaatcatattgatgatgcagatattttacttttaggtccgcaagtgcgctacttgcttcctcaaatgaaaaagttaggagaggaaaaaggaattccagtagaggttattaatccggttcattacggaatgtgtaacggagaagaagtgctaaaatctgctgagcagttagtttcgaaa

SEQ ID NO: 35 - DNA, 1353 bp, Bacillus aryabhattai
  source 1..1353: mol_type = genomic DNA
agcaaatttaatagtatgcttgaaaacaaagtaatgcctatcgccggaaaaatggcgggtcaacgccatttacaagcacttcgagacggtattgttctgacaatgccgctcattattatcggttcagtatttttaattttatccaatctaccgataccaggttacaacgactttatggcaggaattttcggaaaagaatgggcaacaaaaatggcctatccagttggagctacattcgatattatggcgctgcttgcttgtttcggaatagcttatcggttagctgaaaaatatggggtggatgctttgtccgcaggggcaatttcacttgctgcttttttacttgctactccgtataaagtgtcctttcttccaagtggagccaaagaagctattttagtagacggcggaattccaactgctttaatgggcagtaaagggctctttgtagccatgattgtcgcgattctctctacagaaatttacagatggtttattcaaaaagatattgtcatcaagatgcctgatggagttcctcctgctgttggaaaatcgtttgctgctcttattcctggctttacggtaatagcatttatttggattttacgtttgttagtagaacaaacgcattttggaagtctgcataatatcgttggagaattgcttggaaagcctttaggtgtgctaggcggcagtttaggcggtacgctagttgccgaacttgttattatgctcatgtggtcttgtggacttcatggagcgaatattgtcggtggaattatggcccccatttggtatggagctatggatgaaaaccgaattgcatttgctaatcatgaagcattacccaatatttttacacagcagttctttgatatttggatcaatatcggaggcagcggagcaacgttagctctagttgtagctatgattttaaaagcacgcagtcaacaaatgaagcagcttggtaagttaggaataggaccagctctttttaatatcaatgagccgatcatctttggacttccgatggtgatgaacccaatgatgcttattccgtttattattacccctcttgtgacggtagtagcaacctatattgcgatgtcgacaggactggtagcaaaaccagcaggtattgcggttccttggaccatgcctccgattatttctggctatttggcaacaggaggaaagttatcgggtgcagttatgcaagctattaatatcggaatctctgtttgcatttattatcctttctttagaatgtgggataaacaaaaattcactgaagaaaatggtatacagcctgccgctgaaggggtaccaagtgatacaaagaaagatattgtg

SEQ ID NO: 36 - DNA, 1323 bp, Bacillus aryabhattai
  source 1..1323: mol_type = genomic DNA
acaaaaggtattaagattgtaacgattggcggaggttcaagctatacgcctgaactaatagaaggatttattaaacgatacgatgaacttccagttcgagaactgtggctagtagacgttgaagaaggaaaagaaaagctagagattgttggtaatttagcgaaacgaatggtgaaaaaatcaggtttgccaattaatgttcatttaacattagatcgaagagaagcactaaaagatgcggatttcgtaacgactcaatttcgcgttggtttgttagatgcacgagcgaaagatgaaagaataccgcttaaatacggggtgcttggtcaagaaacaaatggaccgggcggattatttaaaggacttcgcacgattcccgttattttagatatctgccgtgacattgaagagttatgtccagaagcttggctgattaacttcacaaatcctgcaggcatggtaacagaagctgtgctccgctatagtaacattaaaaagattgtaggactttgtaatgtaccaatcggaatggaaatggggattgctaagctgcttgatgtagatcactctcgcattcgaattgattttgctggtttaaatcacatggtctatggattagatgtatatgtagatggtgtaagcgttaaagatcaagttattgatttattgagtgatccaactaacagcagctttgtgaaaaatgtagcgggacttggatgggagccagaatttacaaaagccctaaatgcgttaacgtgtccatatcacatgtactattataaaacgcgtgaaatggtagaaaaagagcttgttaattatgaaaaaggtgaaacgagagcagaagtcgtaaaacagttggaaaaagagctgttcgaactgtataaagatcctaatctagacattaaaccgcctcaattagaacagcgcggcggagcctattatagtgatgcagccgttcgattgattacatccatttacacggataaaggcgatattcagcctgtaaatactatcaataatggtgccatagcaagcattcctgctgattctgccgtagaagtaagctgtatcattacaaaagatggaccgaagccaatttcagtcggcgaccttcctgtacctgtgcgaggtcttgttcaaacaattaagtcatttgagcggattgcagctgaagcagcagtgaccggagattatgataaagctattttagcattaaccattaatcctcttgtggcttctgataagctagctaagcaaattgtcgatgagatgttagaagctcataaagattacttgcctcaattttttaaacctgttcaagct

SEQ ID NO: 37 - DNA, 1311 bp, Bacillus aryabhattai
  source 1..1311: mol_type = genomic DNA
aaagtagtgacaatagggggaggatcaagctacacaccggaattggtcgaagggtttattaaacgatatgatgagcttccaattcgagagctatggcttgtagatattgaagaaggaaaagagaagcttgaaattgtaggagcacttgcgagaagaatggtcaaaaaagcaggtgttgatatggaaattcatttaacgcttgatcgacaaaaagcgttgataaatgctgattttgtaacgacacagcttagagtaggtcagcttgatgcgcgtattcaagatgaacgcatccctcttaagtacggaatgattggtcaagagacaaacggggcgggaggattatttaagggcttgcgaacgatacccgttttgttgaaaattgccgaggaaatacacgaagtttgtccaaacgcctggcttattaattttacaaatcctgcaggtatggtaacggaggcgctgcttcgctacggaaagcattctaaagtagtaggagtctgtaacttgcctgtacatatgacaaactccatcgccggcttgcttcaaatagaaaaagaagatgttcacattgaatttgcagggttaaatcacttagtatatggactgcatgtgtatgcaaaaggaaaggaagtaacgcaagaagtcattcggcgattaagtgatccaaagtcccaagtaacaatgagaaacatagctcctattccgtggcaaccggactttttggaagctcttggcgtgattttatgtccttatcaccgctactactacaaaacaaaagaaatattagcagaagagcttctggcctttcagtcaggtaccacacgcgctgaagtggtcaaagcattagaagccgagctttttgaactctataaaaatccaaatctagaaagtaagcctccgcagctggaaaaacgaggaggcgcttattacagcgatgccgcatgcaatttaatttcatccatttacaatgataaaggtgatattcaaacagtaaatgtaagaaacaacggcgcgatttcttcactcccaaacgaatctgcggttgaagtaaactgtgtagtaacaaagcaagggccagtaccactcgctgtcggtgaacttccggtagaagtgaatggacttgtgcagcaaattaaatcgtttgaacgagtaggggcagaagctgcggttacaggatcttatgaaaaagcactggttgcgctgagcattaatccgcttgttccaagtgacgagctggctaagtcggtcttagacgaactgcttcaagctcatcgcgagtatttacctgctttttttaaggaagtgaacgca

SEQ ID NO: 38 - DNA, 1323 bp, Bacillus aryabhattai
  source 1..1323: mol_type = genomic DNA
acaaaaggaattaaaattgtaaccattggcggaggttcaagctacacgccggagcttgtcgaaggatttattaagcgctatgacgagctgcctgtacgagaactttggctagtagatgtagaagaaggaaaagaaaagctagagattgttgggaatctggcaaaacgaatggttgaaaaagcaggagtgccaattgatattcatcttacgttggatcgaagaaaagcgttaaaagatgccgactttgtcactacacaatttcgcgtaggattgttagacgcaagaatgaaagatgaacgaattccgttaaaatacggcgtgattgggcaagaaacaaatggaccgggcgccttattcaaagggctgcgcacaattcctgtgattttaagtattgtgaaagatatagaagaactgtgtccaaatgcgtggcttgtaaactttactaatccagctggtatggtgacagaagccattttacgctatagcaatcataaaaaagtagtgggcttatgcaacgtgccaatcggtattcgcatggggattgctaaagcactagaagtagacgaaagccgcgtacaggttagctttgccgggttaaatcatatggtctttggattagatgtctttttagacggagtgagcgtgaaagatcaagtgattgacgccatggcagacccgacgaatgcgatgtctatgcgaaatatcaaaggagtttcatgggatcctgactttattaaaggcctaggagttattccatgcggctatcaccgctattactataaaacaaacgagatgctgcaagaagagcaaaatgcagctgagctcgaaggcacacgagctgaagtagtgcgcgcgttagaaaaagacttatttgagctctataaagatcctaatcttgcaattaaaccgcctcagcttcaaaagcgaggaggcgcgtactacagcgatgcagcctgcagcttaatttcatctatctataatgataaaggcgatattcagcctgtaaatactattaacaatggtgcaattgccagcattccaaatgattctgctgttgaagtgagctgcgtaattacaaaagaagggccaaagccaattgcagttggtgatttacctgtagctgttcgagggcttgttcagcaaatcaaatcttttgaacgcgtcgcagccgaagcagccgttacgggagattacaattccgcgcttgttgcaatgacaatcaatccgttagtcccatctgatactattgccaagcagctcttggacgaaatgctagacgcgcataaatctcatttgccgcagttttttaactcggtgaatgtc

SEQ ID NO: 39 - DNA, 1944 bp, Bacillus aryabhattai
  source 1..1944: mol_type = genomic DNA
cttaattcaagaatgatcgtcatattaagagaactaatgtccgtacagtctacattatcaaccgttacgagcgaatatttggcaacagtaaatcaagtatcttctcgaactattcgaacggatataaaacagctagatgacattttgtctcagtacggggctaaaattaaatcagctcgagggacaggatatgaacttagcgttttagatgaaaaacagtttcatcagctattaaaagaactttcagctaatcaattatttgatacagcgtctattccaaccgttcctgatgaccgcgtgcattatttgatcaaaagattgctgctagcagacggatatttgaagctcgaagatttagcagacgagttgtatattagcaagtcaacggttcaaaacgatttaagagatgttaaaaaacagctgtgtaagtatgatatccttttagaaaaacgtcctaactatggcttgaaagctaaaggggacgaaatgaaaatgagattttgtatgtctgagtacatattcaaccgaaaagataaagaagtagatatggtacacaatgaaatatctatcctgccccaagaatcaatcttaacgattaaagcccacattttaaatcaaataaaacgatacaatattatggtttcagacattggattaaacaatttaatcattcatttagctatagcttgtaagcgaatacaagaagaaaattttatctctttgtattcagaagacctgcttgagattatggagcacaaagaatatgaagtagcagctgaaattgtacgagaaattgaaaaggcattcaaggttcgttttccgaagtcagaaacagcgtatatcgccattcatttgttagggacaaaaatgctcaataatggcaatcatgaagaaaaagaagtgaagcagtttattgatacaaatatttatgaattaacagcggctatattagaagcaattgaaaaggagcttcatttagaaattagtgaagattcagagctgatccttggtatgagcttgcatttgaaaccggctatcaaccgctataaatatggaatgaatctacgaaacccaatgcttgaagggattaagagccattaccctctggcttttgaagctggaattatagctggaaaggtactaaaaaatcgccttgaaatcgacatgcatgaaacagaaattggctatttagctttacatattggaggtgccattgaacgtcggaaaattatcaatcagcctaagcgctgtttgattgtatgcgcatcgggagtagggagtgccaagctattatcatataagctgcaggctacgtttggattaaatctgactattttaggaacaacgcagtattataagcttcaccaaatgtcactgcattctttagattttattgttagtaccattccaattccagatcatcttcctgttcctgtagtagaagtaagtaccattctaggaaacagtgattttaaaaaaattgaatgtttaatgaagaacaaaccgcagccttcgttagaatacacgaaaaaagaattagtatatttacagcaagaatttcaaacaagagaagaagtgcttcatttcttaggagagaaattaactcatctaggactagcttcttctcaatttatagaatccgtttttgaaagagaagcagcatcaccaacaagttttggaaatctcgttgccattcctcatcctatggttcctcaaacggatacgacgttttgggccatttgcacgttgaaaaaaccgattatgtgggaaggaaatcttgtacaatttatttgccttttaagtgtgaaagcgaataatcaaggcgatttgcaaaaaatgtacagtttattagttcaagttgtagatgataagcaggtcgtgcagcgtttagtaaagtgtaaggattatgaggaatttatagaggtatttatgaaaaatatcaga

SEQ ID NO: 40 - DNA, 258 bp, Bacillus aryabhattai
  source 1..258: mol_type = genomic DNA
ggtgttttacaaaatatttggccattacttttaatgtttgtcattttctatttcttgttaatccgtccacagcaaaaacgtcaaaaaaacgtacagcaaatgcaatcagcacttaaaaaaggtgataaggttgttacaattggcggtatgcacggcttggtagacgcaattgatgagtcacaagttgtggttcgcagcactgacggttcaaaattaacgtttgatcgcaatgctattcgtgaagtaagaagcgtagat

SEQ ID NO: 41 - DNA, 2346 bp, Bacillus aryabhattai
  source 1..2346: mol_type = genomic DNA
aaaagctggaaaaagattttggggacatcttttcttgccacaactgttacattaagcggagtggggccagtacttatgaatggaagtactgcttcagctaaaaccgcacaatcggatgaacttccgagttcaccaattgatatgaatacagtaccggaagaaagattggctaacgcactgaaaaaccaaggagttatttctcaaagtgcaacaccgcaagaagtgaagaaagccgttacttcttacataaacaaaaaagatgcacataaagagagcatgaagggacataaagctagcaaaattgatttgaaagcgaaagaaatgcaaacaaaacaaaaagaaaagtttcaacgaggcgaatttgctaacattaagtctcaaggaaaaaattcggggaacggggtaaacgttaaacctgcagctcaggcaaactataaaggacaagtgagaaaagataaagttcttgttcttttagtcgaatacgcagactttaagcataacaatgtcgttcaagaacctggttacatgtatgcaaaagattttaaccgaaatcattatcagcaattaatgtttggaaagaaagattttacgctatttaacggaaagaaaattaaaacgtttaaacaatattatgaagaacagtctggaggtagttacacagtagatggaacggtttctgactggttaactgtacctggaaacgcaaaagaatacggagcagacaacccagcgggaggaaacgataatgcgcctggatctaaagggcctcgtaatcttgtaaaagaagcgcttgctgctgcagcaaaaagcggcattaacctttcagattatgatcagttcgatcaatatgatatcgatggagacggcaaccgcaatgagcctgacggcatagtggaccacttaatggtcattcatgctggaactgggcaagaagcaggtggaggtaaactaggaaacgatgctatttggtctcatcgctctactttgggacaagtgtataaagttcctggaacaaacatggctgcttatgattatacaattgaaccagaagacggagcagttggcgtttttgctcatgaatttggccatgatttaggtttgccggatgagtatgatacacagtatacgggtcaaggtgagccggtaggcgtttggtcaattatgagcggagggagctgggctggtgaagtagctggaacagaaccgacaagtttctccccgcaaaataaagagtttttccaagcaaatatgggtggaaactgggcaaatattcaacaaatcgacagcaaaaacattacaaaatcgggctctgtgagcgtggttgatcaaagcgtgacaaaatctaaacgaccgggtattgtgaagattaatttacctaaaaaagctgttcaagggattaagcctgagtttggaagcaactactactacagtaaacgcggagatgatctgcatacgacaatgacgacgccagagtttgatttaacaacggccaaaacagcgacgttcaattataaagcgttctatgaagtagaggcagaatacgattatttatatgtaaatgcgattcttcctgacggttcaaaagttctgttagatacaattggtgataaagcgaataacgcagacggttccgcagaaacttccggcggaaactgggaagataaaagctatgacttgacgcagtttaaaggtaaaaaaatcaagcttcaatttgaatacgtaacagacggaggacttgctcttcaaggatttgcacttgataatgccagtttagtagtagatggaaaaacggtatttacggacgatgctgaaggagcgccaaaagtaacgttagatggatttgaaaagacaaacggcatcagctataaagacaactattattaccttgagtggagaaactatgcaggcagcgataaagcacttcaattcgcgcgaggtgcaaaatataatacaggcatgctgctttggtacgcagatgaaagctaccttgataactgggtaggacaacatccaggtgaaggattccttggcgtggttgattcccatgctcataaagttctttactttaatgtgaatgggattagcacgacagcaaattcaagccgatatcaaattgcagacgcggcgttctcatttgataaatcacctgcttggtcatattcccataaaacgtggggaactatttcatctaaaggatcaaatggagttacaagcttcaacgacaaaaaatcgtatttagatgaaagaattaaagatgcaggccgcttgcttcctcaaaacggattaaaattagacgtaattggtgaagcaaaagacaattcagccggtgctgtatggattcataag

SEQ ID NO: 42 - DNA, 1149 bp, Bacillus aryabhattai
  source 1..1149: mol_type = genomic DNA
aaattatacgctctgtgtaaaaaaacatgggcaatgtccctgcttatttttgttcttttagtaagcttaactcctgttagtcaagcagccgcgtttttatctgttgcacaagctcttgagaatcaaaacaacagtgtacaaacggttaaaggatatgtagtaggtcaacctacaggcacatctacagttattacgagcaattacccaaacgattatgcacttgctcttgctgacagttcaaatgaaaccaatacagacaagatggtgtacgttcaaattccttcaaatttgcgaagtacatttggacttcaaagtcactcggagttaaaagggaaaagtcttactgttacgggcacactcactccttatttttcgcactctggaataaaatcagttacttctatcagtacagaaacaggaggaactgatcccgctccagatcccacagagccaaccgttccagtagaagactattatcgtactgcagctgaaaaaacaggaaacagcttaaagacagagcttcataacattattgatcatcacacagaactgtcgtattcagccgtatgggaagctttaaagaaaacggatgaagatccagctaacgcaaataacgttattttgctctacacagggcgttcacaggctaagggtacaaatggaggcggagtggacgattggaaccgtgagcacgtctgggcaaagtctcatggggattttggaacagctatgggacctggaacagatcttcatcacttacgtccaaccgatgtgtctgtaaacagtacgagaggaaacttagattttgataacggagggacggaacatagcgaagcactcggaaattattttgacagtgattcatgggaacctcgagatgaagtaaaaggcgacgtggctcgtatgttgttttatatggccgtgcgctatgaaggcgatgtaagtgatgagcctgacttagaacttaataacacagtgaataatggcactgctccttatcacgggaaattatctgtgcttcttcaatggaatgcccaagacccagtagatgatcgagaacgacgtagaaatgacattatttattcagactatcagcataaccgcaatccgtttattgaccatcctgaatgggtgaatgagatttggaac

SEQ ID NO: 43 - DNA, 1299 bp, Bacillus aryabhattai
  source 1..1299: mol_type = genomic DNA
tcagagaagaaaccgacttacttagaagaacagctcgaagctgtaatgacacgtcataataatgaatatacatttatttttcaaaaagaaaaaattcgacttaatcgttctgttgagattgaaatgttaaaggatgtaaacacggcttttcaaaaagaaatcatgatgagcgatgatgaacttaaaataacaattaagcctacttctgaatttcaaccttttacagcattaagaggaaaagaagaacagcaaaaatggctgtttgcagatcagcttatcaaacaggtaaagcagcattctttttcaaggctgcatttgttgatttgtccagaaaatatcatttttgataaaagtttaagtccggcttttttacattatggagtcaaagaaagtttacctccttatgagtctaacgaggaacaaagctttcaagagttaaaggctacgatagcagcggccgttgactcatcgcatacatttgaacagtattttaagttatatgagaccttgcaattaaaagaagacgctaaaaaaataatgcatatcagcaatgaggaagagttgagtcaatttgttcagcataagctaacccaaattgaaaagcatcaaaagacactcgttcaacttcctcaaaaacgctggactatttttaagttttcatctgtgggtatacttgtcttattaattccagctcttatttatactttttactcttacttttttgctcagccgaaacaagaagcatttgtagaaagcaacgaatactttcttgaaaaaaactacagcaaagtagtcgacacacttgaagattatgacattgaaagcatgcctaaaattgtgcaatacgaaatagcaacttcctatcttgttaatgaaaagttaggggaggagcaaaaagcaaatgctttaaagactattacgcttcagtcagatcctcagtattttgcgtattggttttacattggccgaggagacaacaaaaaagcgctagaagtcagcaggttgttagaagaacgttcattaattatctatgcactaatgaaatacgaaggtcagctaaagacagacaacgatttagcggctgataaaaagcaagaggagcttgataaagtagcgaacgaacttgaagaatttaagaaagaaaatgatcagcaagaagcggaagcagccaagcagcaagaagagaaacaagctgaggaggcacaggcggccaagcagcaggaagaagatcagaaagctgcagaagccgagaaaaagaaacaagaagagcagaaaaaacagcaggaagaccaatcgaag

SEQ ID NO: 44 - DNA, 4473 bp, Bacillus aryabhattai
  source 1..4473: mol_type = genomic DNA
aatatgctttgggtagttagtgggcaaacgtatcagcagctgcctgtagattttaaaacctttcgccaagcaacggttggaaacactcagcatcaaacttttacgttttcctatccgtttgagaaagggcaaatgacgctgtcatctgaaaagaacggccggctcttaatttcctacggagaagaaaggatagcagagctatctgtttctgagtcgtttgtatggcgagaacaaggacatgctttaacctttgtccttacagagacggctgttaaagaagctgtctattatattgattacggatcagaagtttcgttttccacaagtgatgaagcagctgatgtgtatcagcagaaacaaaagtgtgtagtagagccaaaagaaggattttacttatcaaagataaacgggaagtggaaggtcttttataaccaagaaccgctttatcttaacggacaccgactagaagagaatacgaaccttcagcccggagctcttattttatggtcttctatgacgattcaattgatcgaaaaagatttgctgaagattcaaagctactacgattatgcatctaagcttccagctacggcacagcctatttctgaaatgaaaaagaagtacccggtttatagacggacaccgcgcatgatatatgatttgcctaatgataaggtaagcttgtcctttccgtctcaggacaatgatgaaaataatcgaagcttatggcttactattttaccgccattagtcatgcttttagtcatgggtacggtctcgattattcagcctaggggaatttttattattatatctgtcatgatgtttacgactacgctgattgtatctaccgttcaatacttcaaagataaaaaacagcgcaagaagcggcaggagcgtcgtcgtcgaatttatacaaagtatttagaaaacaagcgcgaggaattacaagaactagtatcagttcaacgagatgtattatcttatcattttccgacgtttgaacgaatgaaatatttaacagaacagatttctgatcgaatttgggaacgaactgtagaaagcagtgattttttacaggtgcgccttggaacaggaacagttccgggaagctaccaaatttctttgaatagcggagatatggctaatcgagaaattgatgatttgcttgagcagtctcagcacatggaaaaggtctatcgagaagtgaaaaatgcccctgttaccgctcatttaggtttaggctctatcggtctggtaggaaagatgaaaacggtgaagaatgaactgcatcagcttgtaggacagctggcattttcccacagttatcatgacctaagattcgtattcatttttgatgaagaagaatataaagagtgggaatggatgaaatggcttcctcattttcagcttccgcattcttttgcgaaaggtttcatctataatgaacaaacgagagatcagcttttatcttctatttatgaaatgctgagagaacgcgatttagatgaagcaaaaggagaaggggttttcttgcctcactttgtttttattattacgaatcagcagttaattgcagatcacgttatcttagagtatttagaaggatatcaccctcaccttgggttctctgcgatatttgcggcagagacaaaggaaagcttaacggagcatgttcatacactcgtccgttatattaacgaatccgagggggatatattaattcaaaagcaaaaagctgtgaatattccatttcagcttgataaacacacgcttgattataatgaaagatttgcaaggatgctgcgaacattggatcaccaagtcggcatgaccaactcaattccaaatagcgtttcattcttagaattatttaaagcaagagaaattggtgagttggatatcgtgcaaaggtggcaaagcaatgaatcagctaagtcgctagcagttcccattggtttaaaaggaaaagaagatattgttgaactaaatcttcatgaaaaagctcacggccctcatggattacttgcaggtacgacggggtcaggaaaaagtgaattcttacaaacgtacattctttctctagcggtgaatttccacccccatgaagttgcgtttttattgattgactataagggcggaggaatggctcagccgtttaaaaacatgcctcatctgcttggaaccattacaaacattgaaggaagtaagaattttagtacaagagccctagcttctattaaaagtgaattaaaacggcgccagcgcttgtttgatagatatgaagtcaatcacattaatgattatacagatttatataagcaaggtatggcagaagaacctctgcctcatttattcttaatctcggatgaatttgccgagttaaaaagtgaagaaccagagttcattcgggaattagtcagcgcggcgcgcattggacgtagtctgggcgttcatttaattttagcgacgcaaaagccaggcggtgttattgacgatcaaatttggagtaacgcgcgctttaaagtagctttaaaagtccaagatgccgctgacagtaaagaaattttaaaaaatgcggacgctgcttctattacggtgacgggtcgaggctatctgcaggttggaaataatgaagtgtatgaactgtttcagtcagcttggagcggcgcgccgtacatggaagatacgtatggggctgaagatgaagtcgccattgtcacagatctcgggttgattcctttatctgatgtatctactcagcagacgtcatctaaacaagcggaagcagaaattgatgttgtggtgcaaaaaattgaaaaaacgcagcaggaactagctatcaaaaaattaaatagtccatggctgccgcctcttaaagaaagattgcttcgcagtgagtacaaagagaagcgagacggcttattcccaattggcttgattgatgaaccggaaaagcaaagccaaacggtatataactatcagctgatggacgatggaaacattggtatatttggttcgtcaggatacggtaaatcgtttacagtcatgatgttgcttcttagtctagctgaaagacagagcccggagcagcttcattattacatttttgactttggaaacggcacgctgctgccgcttcgtcagctgcctcatacggcggattatttcttaatggatcaaatgcgtaaaattgaaaaattcatgacgattatcaaacaagaaatcgcaaggcgaaagcagttatttcaacagcgggaagttagcaatatcaaaatgtataatgctctaagttcagaagagcttcctctaatttttatcacgattgataattttgatcttgtcaaagaagaaatgcaagatctagaaatgcagttcacacagctcgtgcgtgacggtcagtctttaggaatttatatgatctttaccgcgacgcgcgtgaattctattcgtcagtcgcttatgaataacttgaaaacaaaagttgttcattatttaatggatcattcagaagcgtattctattttaggacgtacgccgtatgcgctagagtctattccaggccgggctattatcaaaacggatcaaccttattttgcgcagctgtttttacctgcggaaggaaaagatgattttgaaatctttgatgcagttaagctgcacattcaacgattaaagaatcaatatgctgatgctactgttccggagcctgtaccaatgctgtcatcgaaacttgtacttgccgagtttatgacggacagaagggttattcgggaaaaaggagtcattcctcttggattagatgaagagtttgttcgaccggtgcatatgaactttacgaaaaataagcattgcatcgtcatggggcagacgcaaaaaggaaagacaaatgtagtgaaggtcatactgcaaacagcgcttgaacaaggcgtgaagcaaatcggactatttgattcggttgaccgcggtttatcaagctatgcagctgaagagcaagttgtttacatggaaacaaaagaacagattatcgactggcttgatttaactgaagaacgattgcagcgacgtgaaaataagtatttggaagcagtgcaggaaggaacggtacacagtctttcatttgccccgatcatgcttgtagtagacggatttgtacgattccagcaaaacttagacagcgtcctgcaggatcgtattacaaaattgatgaaaaattatagtcacctgggctttaacttgattgttgcagggaacaacaatgagttttcaaaaggatttgacgcgttaacaacagaagtgaagcaaattagccaagccgtgttgctgatgaagaaaacagagcaaagcttatttacacttccttatgaccgaaaagaagccgatgttaaccctggcttcggttattatattaaaagtggaaaagaaacaagaattcaaatcccgctatgtgtaagtgaaaggaagatcttaaca

SEQ ID NO: 45 - DNA, 312 bp, Bacillus aryabhattai
  source 1..312: mol_type = genomic DNA
gatgcaattgatgatctctttgacgacatagagcgaagaagaaaaagtaaagaatatagtcgagatgcagatcaattagaatcatatttacatgaagttcaacgtatcatggaatttcttgaagagggtatctatctgtttcaaaattcgcaccaacaatatgcgtcagattggtcagggcgttctaagagctcatatgaggatatttataatgatattacgcaatctacttttcatctgtacgacgtaagggatgaattgtttcaaactttacgcttagaaatttcacgattaagagagttagcatcggcg

SEQ ID NO: 46 - DNA, 594 bp, Bacillus aryabhattai
  source 1..594: mol_type = genomic DNA
gagataaaagtaaatccagaggtgctgcagacatttgcagagcgaattcagcagtctcattcacagacggctgatgcacttcaaaaactaagctgggaagctacaaatctacatttcttgtctgttgcagatattgataaagtgctggacttgcaggaagagttacagcatatgattcgtaaacttgattcgctaacggaaaacgcacatttattagtaaaagacactgcagaacaaatgagaaaagcagatggagagctagaaggctcttcaggctggacggagttttggtcgagcgtgtggagcgatgggagcacccattcacaagatacgtggaatgatataaagaacttaaaagaatggaatacgtatgacaatatgcatagaacgattgaaaaacctttaggtactcttaatgttatgtggaattcgctttctatgtcttgggaagaaaaagtagtgaatggttctacatgtagtcgaacggaattttttacttatggcgttattcaaatggggttgggcattttaagtcaggaaatgaatccttcaaacattatgtttagtgacagcacagaggaagtgaagagagaa

SEQ ID NO: 47 - DNA, 387 bp, Bacillus aryabhattai
  source 1..387: mol_type = genomic DNA
aaatatacatttaacttattagaaaaatcattaattgaagcaatgaaagataataagaaaattgctcaattttataaaactttaatgaatagcaatttatggctagttgttagttcagagcatggagcatcagatatattaattgcagagcactacgttcagacgatgaaaagcgcagcgtaccgatatttacctgttttttcgtcaaaatatccgattgaacagatgctgaaagatgaaaagaccgtatgcgtatcatttaaggatatgctgcctgtattaaatgaagaaatagcgatattacttaaccctgacacgcctctctcaaaattattcatccccgaagaaataaggatgttaaaagaagatcaaaattattttaagcag

SEQ ID NO: 48 - DNA, 3168 bp, Bacillus aryabhattai
  source 1..3168: mol_type = genomic DNA
accgaacagcgtaagtatacacttaaagtcatattagctgttgttttaattttggctcttccttctttctttttccagtatattggagacaacccgctgcaagtcacagaaaactcaacaagaacgattgcggttgttaacgaagataatggagtaaaagaaaatagtgaaaaagcgatagaatttggagaaaaagtgactcctcttttacaggaggggtcggattatcaatggaccgtgcttggacgaagcgcagctacaagcggtttacagaatgggaaatatgatgcggttatctatattccctcgaccttctcacaaaatattttatcgtatcaggaaaaaaatccagaaaaagctactttggaatacaaagttcaaagtcagcttaacgcagttaataaggaaaaagtagtaggggaattagaagatgctactacaaaggttaataatgaaatgtcctctttgtactggagttatgtatcacaggaagtagataaggtacgcggacagttcgataaaattctaaataaagaaatcgcctttcaaaaaacaatggtcgctttttataaaccgaattcaaaagatttagcaggtgaattaaatgatcaaaagaaaattcttcagcaaatccaagataatgtaaataaagcagaaaaaggttcaagcgacagacaaggcgacgtagaacaggttgaaaagaatttaaacagcttcattgaatatgtaaatcaatatcaaacatatcaagaaacacagaaacaaatgcttgaaaaaacgcaggctcaaagcgtcgctgcgattcaagaagagttgtcttcccttgaatctaatcagccaagctctaagcaaaattttaatgagcaggccaatcaagtgtttagtggcttatcttccattcaacagcagttggatcaaaatcaaaaagccgtcggagaacttggagaacaacaagcggataacgtcagtgaacaagaaaaagctctgcaagatttgaacggccaaatagtcagttcatataaacagcagtctgagcaaacaaccctaaatcaaattgaaagtaagctaaagccgcttcgtgcgcagctagagacagcttcatcaaatgagggtggcggaggctcaacggatccaggagaagatggcgaagctcagccagatccaaatcaaggcggagagacgccaacggaccctggggaagataacggaactcagccagatccgaatcaaggtggagagactccgacggatcctggagaagatgatggaactcagccagattccggtcaaggcgaaggaacgtcagaaccaattaaagtggatttatctcagcaaacagcaaagcttgatgatatatcaaaaggacttgaagacttagaacagcagctccaaggaatggtgaacgaagcgcctgaagcgcctgaagcacctaagacatctgagggaacggaaacgcctgaaacatcggacacagcggatgcatcaggtacatccaatgctgtgaaaagtgcgcttgaaaaagtggctaatctgaaaacacaggtacaagcagtcaaagatgagctgcaaaatgtaaccgttcagccggaagcttcaggagaaaatacggatgcgctgaagcagaaaattgccagtttggaagcagacgttaagtctttgacagcacagaaaaacaaagcagagaaagacttagaagaagcaaccaagtcgtcgagcggtcaaattgctaagcttcaagaagaaaatgaacgattaaaaaaagagaatgaaaagttgaaagaagacggcaaaaagccttctaaaggtagtgtggatgtagacaatctcattacgatgattaaaaacaaagaagataaaattttatcttctccaaacctttcagcttctcgcaaggaaaagttgagcaatacattctcaggtaagattcaaagcagcagtgcagaagatctgctaaagtactacggtgatttatcgcagtatgatttgactttagctcaaataggaaatacagacatacagaacagcgtccttaacaatgactctattaaaggaggtttgcagcaggcgttagctggcaccgtctcttctcaagatgcacgtaagcaaatccaacaaaacctgtctctaacaaaagaagagttagcacagttccaaagttcagtgcaagattttgctgatcaatatggacaaacagttgatgctgaacaagcatctattatggaagagctatcagctattcaagaaaaagcaaatgcaatgaatcaagagcttcaatctagcgcaggtgaagatcaagcattagaaaagaaagaagctccggacgtaacgggcttaatgacgctgcagcaaagcatgggtcaagagttgaagggaatgaacgaactcatttcatctttaggtgaaagacaagctaatgttgtcacatacacagatgaattgcagaagcgtgtaaatgaagtccaggataaagctgacacgcttaacagcaagtgggcccaaaacgtggattcaacgaagttggttcgcggccaggtataccgtttgttaaataatacattagtagatggacagaataatgattacgtttatcattatttagcgaatcctcttcaagtaagcggtgaagtgccggctgaaaaagtaaaagaggttccaccagttgtcattttggttattatcttaatcagcagcttactaattggctatttcagtcaatactaccgacatgcacctatgcttgtgaaaggtacgctgtttgggttattgaatttgattgtagggttaatggtaagtatctttagccttaatatttattcgttatcgggagaccgggcaatggaatggtcaatctttacaatcgtcttattgcttgcttcatctacactcgtacgtattgctttcttattaggaagcttcgctggatgggtgacgacagtagggttgattttattttatattaccccgcttcttaacttatctatgccaaactttaattacgaagatccagtatctaaagtatatatgtcaattcaatatgatacgcagtctctgtttggacaggcgcttattgtattactgggaatgactgccgttttaagtgttctgccatctattattcgtgctttaaagacgactaaagaggttgaaaatgatgaaacctatagtgct

SEQ ID NO: 49 - DNA, 492 bp, Bacillus aryabhattai
  source 1..492: mol_type = genomic DNA
atgaaacctatagtgcttaataagaagattggaagtattgttcttattttttcacttctttgtttccctatagcagcagcggctgaagctgatgacggaaacgttgaagagattaaaccaaatgtatataaggatgatccgattaatttggaccccgaatctttgctagaaaataaaaaagaagtaggaaatattccggaggaaataaaggatcttacatttgaaaaagaggaagatgcagagaaagagagcttaaaagaaagtctttttgcgaacgcatcagctgaagataatacgatttctgcgaaagcaaaagaatataacttgttttcttctaaaaacacttcctacaagaaagagcgaacagaacaaactgtttctgaaaagtcatcagacctacaatggttttatttattattgattggtgtgggggttgttatattatttgcggttttaataccgcgcattagccctcagccttctgtaaaaaaa

SEQ ID NO: 50 - DNA, 234 bp, Bacillus aryabhattai
  source 1..234: mol_type = genomic DNA
tatatagaagtaacagtagacttaaagcactatacaggtgatcggttcgatttgcgactatcaaactatcattcactcaaaaaagtagtcgagatcgtttggcaagctaaaaagattactcaatcaccaaaggacggtgcgtggatacgtgtttcaaataaaaatcttattttctcgggaaatgagacgatgattaactgcggaattaccacaggtgattgtatcgaaattctc

SEQ ID NO: 51 - DNA, 288 bp, Bacillus aryabhattai
  source 1..288: mol_type = genomic DNA
tcagggattattcgcgtaacaccagcagagcttcgcgacatggcaggtcgttatactaacgaaagcggtcaagttcaagaattagtaagccgtcttgatacaatgaaaaatcaacttcaagacatgtgggaaggtgcttcaagcgaagcgtttgctgctcaatacgaagaattaaagccttctttcgttgaaatgtctaacctacttacaaagattgctaagcaattagatgactctgcaaatgttcttgaagatacagataaccaaatcgctagccaaatccgcggc

SEQ ID NO: 52 - protein, 210 aa, Bacillus aryabhattai
MKHSQTYNMKELKTAVGAIVIEGPVSSKTLQQLDFHEQLTAFRPPEQQKEALIEIANLPEGRIIIARHHHTIIGYVTFLYPDPLERWSEGNMENLIELGAIEVAAPFRSYAVGKNLLIVSMMDDAMEDYIVITTEYYWHWDLKGTGLNVWDYRKIMEKMMNKGGLTWFATDDPEISSHPANCLMVRIGSRVPQESIQEFDSLRFRNRFMY

SEQ ID NO: 53 - protein, 213 aa, Bacillus aryabhattai
MIVEEMMKTDLVTLTPDHTIAEAMKLLDTHKIRHIPIVNDLHHVVGIISDRDVRDASPSILDNTYKSALLSEPVRMIMQKEVITAHPLDFVEEIASIFYEHQIGCIPVTKNKRLVGVVTERDLLHTFIQLTGTNKPGSQIEIKVENKAGVLAEVTSLFGSKKTNILSVLVYPDVDERYKILVLRIQAINPLISINTLKENGYEVLWPKLPEMT

SEQ ID NO: 54 - protein, 387 aa, Bacillus aryabhattai
MTSNAVFIYSDDILSYKFSNNHPFNQLRVQLTYELLREFGALGNQDIVLPRMASDEELQLVHDTSYIQAVKLAGSGELSVEKAADFGLGTEDTPMFPNMHEASAFLVGGTLTAVDYVMTGKAKHALNLGGGLHHGFRGKASGFCIYNDSAVAIKYMQQKYNARVLYVDTDAHHGDGVQWTFYDDPSVCTLSIHETGRYLFPGTGNVNERGHSEGYGYSFNVPVDAYTEDESWLDVYKTSLKEVAAFFKPDVILTQNGADSHYYDPLTHLCGTTKIYREIPKIAHEIAHEYCQGRWIAVGGGGYDIWRVVPRAWSFIWLEMIERKAEGMLPLSWLEKWTKVSPHPVCTKWDDEPNIYQPIPRKPEITEKNAQTLERALYPIRSHTSHS

SEQ ID NO: 55 - protein, 558 aa, Bacillus aryabhattai
MSNADTTETKKGADLLVESLASQNVPYIFGIPGAKIDGVFDVLKEKGPEVIVCRHEQNAAFMAAAMGRLTGTPGVCLVTSGPGAANLATGLVTATSEGDPVVALAGAVPRADRLKKTHQSMDNEGLFKSIAKFSVEVEDAANVPEVITDAFREAVSSPSGASFVSLPQDVMDNETSASTLSVLKAPTIGAANLDAVAQAIHTIRTAKLPVLLLGMRASKSKAVESIRQLIAEHDLPVVETFQGAGAVSRELETHFFGRVGLFRNQPGDVLLKHADVVLTIGYDPVEYPPSLWNSESQRSIIHLDDLQAQADHHYQPEIELVGDIPSTIDILADKLPHLIRSKESASFLKTLKSKLEQPDDQYEFSNDTHLHPLQLIEALQNFVEDDTTITCDVGSHYIWMARYFRSYEPQHLLFSNGMQTLGVALPWAISAALVRPHKKVLSMSGDGGFLFSAMELETAVRLNLPIVHIIWNDQTYDMVGFQQQIKYGRKSAVEFGSVDFVTLAESFGAKGFRVNSPEEFSDTLASAFREQGPVIIDVPVDYKDNEKLNTQLLPDQIN

SEQ ID NO: 56 - protein, 531 aa, Bacillus aryabhattai
MKVTDTLVQCLENEGVEYIFAIPGKETLDFVDSLSNSKQIQFVNVRHEQGAAFMADVYGRLTNKVGVCLSTLGPGATNLLTGISSAKLDHSPVVALTGQAALGRQHKDSHQYMDIIKMVEPATKWGVQIKDAQTLPEIIRKAFKIAQMEKPGPVVVELPEDLATQIVPIKPILPTSIPVSIPILESIQAANTLIKQYKKPFVIIGNGAVRQGAVDELQPFIERLQSPVTHSYMAKGVLSKDHPQNFFTFGFNEKDEVLAGLKEADLLIVIGFDFVERLPKEWNTEKTPILHIDTLPAEVDEYYPIEVELVGDIKQTLRVFNQLDIPSKPWIPSGNLQEQIKQSYHIEEKKIDSNLLPLTIENILHIIDKLSSEKTIVISDVGAHKVSIARTYQPKNPNRLIISNGLASMGIALPGSIGAKLACPNDPAICITGDGGALMNFAEIETAKRLGLSFIIIVLNDSTLKIEQEMMNKKFGKSYGVTFTNPDFVQLAKSFGIKGEQANSLVEFENIMKRALQSLDEIVLIDVLLKS

SEQ ID NO: 57 - protein, 349 aa, Bacillus aryabhattai
MKALRWHGVKDVRLEHIAEPTVSQGKVKIKVEWCGICGSDLHEYTAGPIFIPTEEPHPLSKDKAPVVLGHEFSGQVVEIGEGITNVKVGDRVVVEPIYACGECENCKQGKYNLCNKMGFYGLAGGGGGFSEYASIPVEMIHKIPEVVSYEQGALVEPSAVALHAVRQSKLKVGDKAAVFGTGPIGLLVIEALKASGASEIYAIELSEQRRQKAEELGAIVINPQEYDPVAELHKRTGGGVDVSYEVTGVPLVLTQAINSTKSGGETMIVSIFEKEASIHPQTIVLQERTVTGIIGYRDVFPAVISLMAKGYFSADKLVTKRIKLENVIEEGFETLLKEKEQVKILVKAE

SEQ ID NO: 58 - protein, 237 aa, Bacillus aryabhattai
MSNKEKLTIHQTSTMIALLDGVFDGVVTYKDLAKKGDFGIGTFNQLDGEMIAFDGEFYHIADGKAAPISEDAKTPFAIMTTFYEDISYRVEKEMNREQLETLMNELIPSPNLFYAVRIDGKFKEVKTRTVAKQEKPYPSMVDAAADQPTFTSSDVNGTLAGFYTPEYSSGIGVPGYHLHFIDEYKKEGGHVLDVSVQDVTIQICKKMKLNLNLSETSEFLSANLEDHDVEKEIATAE

SEQ ID NO: 59 - protein, 568 aa, Bacillus aryabhattai
MQTKEPVSTGAQMNGADMLIESLRRENVDVIFGYPGGAVLPIYDKLYGSGMFHVLARHEQGGIHAAEGYARISGKPGVVIATSGPGATNIVTGLADAMIDSLPLVVFTGQVASSVIGSDAFQEADVLGITMPITKHSYQVRNISDMPRIIKEAFHIATTGRPGPVLIDIPKDMAQGDGTFNYEGEIDLPGYQPTYFPNQLQVKKLTDAVAAAKQPVILAGAGVLLSKGSEELKNYAERQNIPVINTLLGLGCFPADHPLHLGMAGMHGTYTANMAIYESDLLISIGARFDDRVTGNLKHFAPNAKIAHIDIDPAEIGKNVPTKIPIVGDAKEVLKQLLLQEGEVGDYTKWHEKLAAWKQEYPLWYQNSADVLKPQKVVELLHKYTNGDAVVTTDVGQHQMWVAQYYTFNNPDRWVTSGGLGTMGFGLPSAIGAQLANKDKTVVAVVGDGGFQMTLQELGVIRELNLPVKVVVLNNQSLGMVRQWQEIFYEERYSHSLIPNQPNLMKLAEAYDIKGLQASTEEEAHAALKEAMETDGPVLLNFFVAPKENVYPMVAPGKGLHEMVGVKP

SEQ ID NO: 60 - protein, 170 aa, Bacillus aryabhattai
MKRIITATVLNRAGVLNRVTGLFTKRNFNIESISVGHTESEDVSRMTFVVNVEHDNAAEQVIKQLNKQIDVLKVQDITDQAVVSRELALMKVASTPASRNELYAVIEPFRATIIDVSRDSLIIQVTGESSKIEAMIDLLQPYGLKEVTRTGTVAVPRGTQKAAAKLSSII

SEQ ID NO: 61 - protein, 193 aa, Bacillus aryabhattai
MKIAVGITGATGAILGIRILELLKKASVETHLVMSPWAHATIKLETPYSVQQVEALADYCYSYQDQAAKISSGSFRIDGMIVSPCSMKTLASIRMGLADNLIARTADVMLKERKPLVLLPRETPLNTIHLENMLDLSKMGAILVPPMPAFYNKPETIEDIVTHIAVRTLDQLGIELPEAKRWNGIKHLSQGGK

SEQ ID NO: 62 - protein, 473 aa, Bacillus aryabhattai
MAYKDFRDFLNTLHKEGQLLTVTDEVQPDPDLGSAGQAISNLGDQTPGLLFTNIYGYNNAKVALNVMGSWSNHALMMGLPKSTPVKEQFFEFARRYEKFPVKVKREETAPFHECEIKDDINLFDLLPLFRLNQGDGGYYLDKACVISRDQHDKENFGKQNVGIYRMQVKGKDRLGIQPVPQHDIAIHLKQAEEKGENLPVSIALGCEPAIVTAAATPLHYDQSEYEMAGAIQGEPYRIVKSQLSDLDVPWGAEVILEGEILAGEREYEGPFGEFTGHYSGGRSMPVIKINRVYHRKDPIFESLYIGMPWTETDYLIGINTSVPLYQQLKEAYPEEIEAVNAMYTHGLVAIVSTKSRYGGFAKAVGMRALTTPHGLGYCKLVILVDEDVDPFNLPQVMWALSTKMHPKHDVITVPNLSVLPLDPGSEPAGITDKMILDATTPVAPETRGHYSQPLDTPLETEKWEKILTNMMQK

SEQ ID NO: 63 - protein, 497 aa, Bacillus aryabhattai
MQNLGAKKVEMSPKVVEFLKGVKQLYINGEWVDSVSGKTFETVNPATGEKLADVAEANPEDIDRAVRAAREAFDHGPWTKMSAAERSRLIYKLADLMETHKLELAQLETLDNGKPIRETSNADIPLAIEHFRYFAGWSTKMVGQTIPVQGAYFNYTRYEPVGVVGQIIPWNFPLLMAAWKLGAALATGCTIVLKPAEQTPLSALYLARLADEAGFPKGVLNIVPGYGKTAGEPLVHHDLVDKIAFTGSTAVGKAIMKQASDSLKRVTLELGGKSPNIILPDADLTKAVPGALSGIMFNQGQVCCAGSRLYVQKSLYDNVVADLVSQTKSIKQGNGLADETTMGPLVSAQQQKRVKTYIDKGIEEGAEVLAGGNIPFDQGYFVEPTIFADVDHSMTIAREEIFGPVVAAMPFDDLDDLIAKANDTAYGLAAGVWTQDLKKAHYIAHGIKAGTVWVNCYNVFDAASPFGGYKQSGIGREMGSYALDNYTEVKSVWINME

SEQ ID NO: 64 - protein, 390 aa, Bacillus aryabhattai
MSTYSFDQLINRTGTQSVKWDKTDLVFGAKDVLPFWVADMDFQAPPNVLQTINERVKHGIFGYTSPTESTANALSSWVEKKHQWTIDHEWITYSPGVVFALSMAIQAFTEPGDSILIQPPVYTPFFNMIKVNDREVVENPLKLAGDKYEIDFADLEQKFAEGIKLMILCSPHNPVGRVWTKEELKKLADLCEKYNVLIVSDEIHSDLIYQPYKHIPISIVSEDAANRTITCMAPSKTFNIPGLQASAIIIPNEKLRTQYVQQQQKQGFSNLNTFGIIGLEAAYSEGEEWLEALLVYLKKNVDLVTRFISENLPELRVVQPEGTYLLWIDCNKLHLSEEELHERLLEKGKIAVEKGEKYSSTYGKGFIRLNIGCSHEQLQEGLNRLKVALT

SEQ ID NO: 65 - protein, 522 aa, Bacillus aryabhattai
MKREKGLFERMSTIDLSKFEKKMIIRNIKPDDIDEILALQKLSFPGMDPWKKEQLESHLAIFPEGQICAEYDGEIIGSCSSLIVNFDEYDDQHTWDDITDEGYITNHNPEGYNLYGIEVMVHPKYRGMKIGRRLYEARKDLARELNLKSIIIGGRIPNYHKHSAEMSPREYVEQVIHHKIYDPVLSFQLMNGFTLMRINPGYLPDDLASNKYATLMEWNNVDYVRRSKRHFKTSFPVRICVVQYMMKQINSFEEFATQVEYYTDVAYDAKADFCVFPEIFTTQLMSFLNEKIPSKAVQRVAEYTEEYIELFTNLAVKYNVNIIGGSHFVEEDNGDIFNIAYLFRRDGTIEKQYKVHITPNERKWWGISPGDKVEVFDTDCGKIAIQICYDIEFPELARIATDKGANIIFTPFCTEDRQGYLRVRYCSQARAIENQIYTVIAGTVGNLPETENMDIQYAQSAIFAPSDFEFARDGIVGECNPNIEMVVIGDVDLEILRRQRQSGTVRQLKDRRHDVYNIKYKK

SEQ ID NO: 66 - protein, 154 aa, Bacillus aryabhattai
MIKIKRDHLMNNEVIELVSSHLQGMNLHSPPESIHALNIENLKQRDITFWSVWEDSQLAGCGALKELDEHHGEIKSMRTATAHLRKGIAKELLQYIINEAQKRGYRRLSLETGSASAFEPARKLYASFGFYECEPFSTYKEDPYSVFMTKKLND

SEQ ID NO: 67 - protein, 318 aa, Bacillus aryabhattai
MSIGQIFLILAPIFFVILLGYLAGHFKKFDKQTSKGLNTLVTKFALPAHLFVGVTTTPRKTLIDEWPFLVALILGIVGFYLIILLVVKYAGKHTLTASSMFALNSAQPTFAFMGIPVLGGLFGAGAVAIPIAITGIVVNAILDPLATILGTVGQRGRKEGEEKTSLFKVTVHSILHGLSEPLACVPLIGVILTLCGFQSPKLLQDMLDQIGSITSGAALFAVGVTIGIGKINFSKGALGIAVLKVAVQPIVMLGIAVWIGLSSDEVTKAILLVAFPGSAVAAMIALRFESLENETASAFVISAILSLITLPLLISWLM

SEQ ID NO: 68 - protein, 318 aa, Bacillus aryabhattai
MEISQIFIILTPIFFVILLGYLAGYFKKFDATTSKGLNTLVTKFALPAHLFVGITTTSKQTLIEKWPFLLALVLGIIGFYVLFLLVAKYVFKYSLTGASMFSLNSTQPTFAFMGIPVLGSLFGADVVAIPIAITGIVVNAMLDPLATIIGTVGQRERSEGDSEESLFKVTIKSILHGLSEPLACVPLIAVILVLCGFQAPDLLSSSLDQIGSITSGAALFAVGVTIGIRKIQFSPAAFGIAILKVAIQPLVMLGIATAMGLSSADIVKLVLLVAFPGSAVAAMISVRFDSLAGETASAFVLSAIMSLVTLPLLISWLM

SEQ ID NO: 69 - protein, 325 aa, Bacillus aryabhattai
MPIDESSVVMTGEKPKLIVIIGPTAVGKTKLSIELAKQLDGEIISGDSMQIYKGMDIGTAKVSEEEMQGIPHYLIDIKEPDESFSVAEFQEIVRKQIDDIHARGKMPMIVGGTGLYIQSVIYDYQFTETPSDEKVRERMEVFIKAHGIDPLYEKLKEIDPDSAAAIHKNNHRRVIRALEVYEVTGKKFSDYTKEQSQELLYDVSIIGLTMDRDVLYERINHRVDMMMQAGLLDEVKSLYESGLHDVQSIQAIGYKELYAYFEGRCTLEEAVEQLKQNSRRYAKRQLTWFRNKMPVKWIEMEVDKFPEKFAEIFTYIAGKLKKEAN

SEQ ID NO: 70 - protein, 509 aa, Bacillus aryabhattai
MNEKQRLESNQVNPADKKSEKDYSKYFQTVYMPPSLKDAKKRGKEEVSYHNDFAIDEQFHGMGQGRKFYIRTYGCQMNEHDTEVMAGIFLGLGYEPTDSVEDAHVILLNTCAIRENAENKVFGELGHLKALKRERPELLIGVCGCMSQEESVVNKILQKHQHVDMIFGTHNIHRLPHILNEAYLAKEMVIEVWSKEGDVIENLPRARKGSVKAWVNIMYGCDKFCTYCIVPYTRGKERSRRPEDIIQEVRHLAAQGYKEITLLGQNVNAYGKDFEDITYGLGDLMDEIRKIDVARIRFTTSHPRDFDDRLIEVLAKGGNLVDHIHLPVQSGSSDILKIMARKYSREHYLELVRKIKEAIPTASLTTDIIVGFPNETDEQFEETMSLYREVEFDSAYTFIYSPREGTPAAKMQDNVPMEVKKERLQRLNALVNEISAKKLKEYEGKVVEVLVEGESKNNPEVLAGYTEKSKLVNFKAPKSVIGQLVKVKVTKAKTWTLNGEMVEETAEVK

SEQ ID NO: 71 - protein, 191 aa, Bacillus aryabhattai
MKRLAVFCGSSKGASDLYIEGAKALGRELAKRNIALVYGGVSVGVMGAVADAVLEEGGHVIGVMPSFLEEREISHKNLSELIVVESMHERKAKMAELVDGFIALPGGPGTLEEFFEIFTWAQLGLHQKPCGLLNINNYYTPLVSLFNHMTEEQFLQEKYRSMALVDTEPHGLLNQFNTYQPPSVKTYITKA

SEQ ID NO: 72 - protein, 192 aa, Bacillus aryabhattai
MKSICVFAGSSAGIKTEYKEAAVSLGRYMAQKNYRLIYGGSRMGLMGEVANEMLQHGGEVIGIMPRGLFSGEIVHTALTELIEVESMHERKATMHELADGYIALPGGFGTFEELFEALCWAQIGIHKKPVGLLNVNGYYNPLMQMVQHAVDEGFSTDSAIRLINISDTPEQLISSMDTYTSPSEDQKWNTER

SEQ ID NO: 73 - protein, 193 aa, Bacillus aryabhattai
MKRIAVFCGSSNGASELYIQGAKALGKELAKRNISLVYGGASVGVMGAVADAVLEAGGNVIGVMPTFLEEREISHKNLSELIVVESMHERKAKMAELVDGFITLPGGPGTLEEFFEIFTWAQLGLHQKPCGLLNINDYYTPLISLFNHMTEEQFLQEKYRSMALVDTEPQGLLDQFNTYEPPSVKTYITKAQS

SEQ ID NO: 74 - protein, 479 aa, Bacillus aryabhattai
MTKMPKDFLWGGALAAHQFEGGWNQGGKGPSVVDVMTAGAHGVARQITETVEEDKFYPNHEAIDFYHRYKEDIALFAEMGLKCLRTSIGWSRIFPKGDEAEPNEEGLKFYDDVFDELLKHGIEPVITLSHFEMPLHLAREYGGFRSRKTVDCFVKFAEVCFDRYKDKVTYWMTFNEINNKMDVHNPLFLWTNSGVSVKPGENAKEVMYQAGHHELLASALAVSKGKEINPNFQIGAMVSHVPIYPYSSNPEDVMLAEETMRQRYFFPDVQVRGYYPSYALKEFEREGYRIPFEEGDEEILRNGKVDYLGFSYYMSTTVKSDVEADNTGDIVNGGLPNGVANPYIKSSDWGWSIDPTGLRYTLNRFYDRYQIPLFIVENGFGAIDTVEEDGSIHDASRIEYLKSHIEALEKAVTYDGVDLMGYTPWGIIDIVSFTTGEMKKRYGMIYVDRDNEGNGSMKRYKKDSFNWYKHVIETNGEEL

SEQ ID NO: 75 - protein, 497 aa, Bacillus aryabhattai
MLTNDTVFKKGFLWGGATAANQIEGAYDVDGKGLSTADMIRFVPKDERTGYSMDVKADYIEGVLNNKVNDRFPKRTAIDFYHRYKEDIALFAEMGFKTLRISINWARIFPNGDDVNPNEAGLQFYDSLFDEMLKYGIEPLVTLSHYETPLELTRKYNGWYSRKLIGFYTSYAETVFTRYKNKVKYWLTFNEINVITHSPYTGGGILVENVPNQSAEQIAYQGAHHQFVASALATKLAHEIIPDVKVGCMLARMTTYPVTNHPDDILLAQHKNDLNLFFTDVQVRGEYPKVMERYFKENNIQIVKEVGDDEIIKQYPVDFISFSYYMSLSVTTQSEEENVLGNMIAGSVKNPHLQTSDWGWQIDPKGLRYTLQDFHNRYGKPLFIVENGLGAHDKVEEDGSIHDPYRVDYLRQHIEQMKEAVKDGVDLMGYTAWGPIDLISVSTNEMSKRYGFIYVDQDDEGNGTLERSKKDSFYWYKQVIQTNGEDLSSSPKVKVSQ

SEQ ID NO: 76 - protein, 206 aa, Bacillus aryabhattai
MNQNSPKYVALTFDDGPSAYTVAILKILKRYNVRATFFVVGAEAEKFPRLIRRIHREKHVIGNHTWSHPDITTLSKRELWKEITSTNAQIEKIIGCSPKLFRPPYSSINDTALADIKELGMTSVLWNVDSQDWREEDPLAICKHTIKNLQEKNLIVMHDGDRYGSGARDQIVTSLPKLIKYLINNDYQFVTVPEFHRVAFKIEGWS

SEQ ID NO: 77 - protein, 395 aa, Bacillus aryabhattai
MKHSYPSYNRPPALRQLYKYLWIFFLLLFLITLIIPTMIVFSTGSGSLAMNKSLTSSISQKSSAVTEDNKISSVSVNKHVSPKESPLSIGWVTGNNNNSSTQDLSAYHNLKVVSPALVSINNQYNLQVNSNPSLINTMHTQDKKIWARFIINNDTKSNVHTFLSNPSKTQEIIKKIYQVSLNNQWDGINLDIENVRSQDRDLFSQFVKSLSNLLNQSKIVLSIDLPPDSKGSNNKYGPFDHKIIGNYCDYIIFMGYDQHWSTDPVPGPVTSLSWLKENVQEFTKTGIPPQKMILGLPAYTRVWQQDNSGKIVKKPATSVQYVEDLVTQNHRNLNWNSTLGEYYTSYTANNRVYQVWLPTVTSFKEYLDLIPQYHLAGSAIWSLDLMSASYWNQIF

SEQ ID NO: 78 - protein, 109 aa, Bacillus aryabhattai
MESIQEYIFKLILHGGNGRSSAMEAIAAAKSGNFSEAREKLTQAAEELNAAHHIQTSLIQGEIRGESTEISLLMIHAQDHLMNAMTVKEMATEFVDLYEAIKISGGITL

SEQ ID NO: 79 - protein, 101 aa, Bacillus aryabhattai
MNILLCCAAGMSTSLLVTKMEKSAQDQSTEYKIWAVPGDSVRNHIDQADVLLLGPQVRYLLPQLKKLGEEKGVPVDVINPVHYGTCNGEQVLKFAVQLVSK

SEQ ID NO: 80 - protein, 101 aa, Bacillus aryabhattai
MNILLCCAAGMSTSLLVTKMEKSAKEKEKACKIWAVPGDAVRNHIDDADILLLGPQVRYLLPQMKKLGEEKGIPVEVINPVHYGMCNGEEVLKSAEQLVSK

SEQ ID NO: 81 - protein, 452 aa, Bacillus aryabhattai
MSKFNSMLENKVMPIAGKMAGQRHLQALRDGIVLTMPLIIIGSVFLILSNLPIPGYNDFMAGIFGKEWATKMAYPVGATFDIMALLACFGIAYRLAEKYGVDALSAGAISLAAFLLATPYKVSFLPSGAKEAILVDGGIPTALMGSKGLFVAMIVAILSTEIYRWFIQKDIVIKMPDGVPPAVGKSFAALIPGFTVIAFIWILRLLVEQTHFGSLHNIVGELLGKPLGVLGGSLGGTLVAELVIMLMWSCGLHGANIVGGIMAPIWYGAMDENRIAFANHEALPNIFTQQFFDIWINIGGSGATLALVVAMILKARSQQMKQLGKLGIGPALFNINEPIIFGLPMVMNPMMLIPFIITPLVTVVATYIAMSTGLVAKPAGIAVPWTMPPIISGYLATGGKLSGAVMQAINIGISVCIYYPFFRMWDKQKFTEENGIQPAAEGVPSDTKKDIV

SEQ ID NO: 82 - protein, 442 aa, Bacillus aryabhattai
MTKGIKIVTIGGGSSYTPELIEGFIKRYDELPVRELWLVDVEEGKEKLEIVGNLAKRMVKKSGLPINVHLTLDRREALKDADFVTTQFRVGLLDARAKDERIPLKYGVLGQETNGPGGLFKGLRTIPVILDICRDIEELCPEAWLINFTNPAGMVTEAVLRYSNIKKIVGLCNVPIGMEMGIAKLLDVDHSRIRIDFAGLNHMVYGLDVYVDGVSVKDQVIDLLSDPTNSSFVKNVAGLGWEPEFTKALNALTCPYHMYYYKTREMVEKELVNYEKGETRAEVVKQLEKELFELYKDPNLDIKPPQLEQRGGAYYSDAAVRLITSIYTDKGDIQPVNTINNGAIASIPADSAVEVSCIITKDGPKPISVGDLPVPVRGLVQTIKSFERIAAEAAVTGDYDKAILALTINPLVASDKLAKQIVDEMLEAHKDYLPQFFKPVQA

SEQ ID NO: 83 - protein, 438 aa, Bacillus aryabhattai
MKVVTIGGGSSYTPELVEGFIKRYDELPIRELWLVDIEEGKEKLEIVGALARRMVKKAGVDMEIHLTLDRQKALINADFVTTQLRVGQLDARIQDERIPLKYGMIGQETNGAGGLFKGLRTIPVLLKIAEEIHEVCPNAWLINFTNPAGMVTEALLRYGKHSKVVGVCNLPVHMTNSIAGLLQIEKEDVHIEFAGLNHLVYGLHVYAKGKEVTQEVIRRLSDPKSQVTMRNIAPIPWQPDFLEALGVILCPYHRYYYKTKEILAEELLAFQSGTTRAEVVKALEAELFELYKNPNLESKPPQLEKRGGAYYSDAACNLISSIYNDKGDIQTVNVRNNGAISSLPNESAVEVNCVVTKQGPVPLAVGELPVEVNGLVQQIKSFERVGAEAAVTGSYEKALVALSINPLVPSDELAKSVLDELLQAHREYLPAFFKEVNA

SEQ ID NO: 84 - protein, 442 aa, Bacillus aryabhattai
MTKGIKIVTIGGGSSYTPELVEGFIKRYDELPVRELWLVDVEEGKEKLEIVGNLAKRMVEKAGVPIDIHLTLDRRKALKDADFVTTQFRVGLLDARMKDERIPLKYGVIGQETNGPGALFKGLRTIPVILSIVKDIEELCPNAWLVNFTNPAGMVTEAILRYSNHKKVVGLCNVPIGIRMGIAKALEVDESRVQVSFAGLNHMVFGLDVFLDGVSVKDQVIDAMADPTNAMSMRNIKGVSWDPDFIKGLGVIPCGYHRYYYKTNEMLQEEQNAAELEGTRAEVVRALEKDLFELYKDPNLAIKPPQLQKRGGAYYSDAACSLISSIYNDKGDIQPVNTINNGAIASIPNDSAVEVSCVITKEGPKPIAVGDLPVAVRGLVQQIKSFERVAAEAAVTGDYNSALVAMTINPLVPSDTIAKQLLDEMLDAHKSHLPQFFNSVNV

SEQ ID NO: 85 - protein, 649 aa, Bacillus aryabhattai
MLNSRMIVILRELMSVQSTLSTVTSEYLATVNQVSSRTIRTDIKQLDDILSQYGAKIKSARGTGYELSVLDEKQFHQLLKELSANQLFDTASIPTVPDDRVHYLIKRLLLADGYLKLEDLADELYISKSTVQNDLRDVKKQLCKYDILLEKRPNYGLKAKGDEMKMRFCMSEYIFNRKDKEVDMVHNEISILPQESILTIKAHILNQIKRYNIMVSDIGLNNLIIHLAIACKRIQEENFISLYSEDLLEIMEHKEYEVAAEIVREIEKAFKVRFPKSETAYIAIHLLGTKMLNNGNHEEKEVKQFIDTNIYELTAAILEAIEKELHLEISEDSELILGMSLHLKPAINRYKYGMNLRNPMLEGIKSHYPLAFEAGIIAGKVLKNRLEIDMHETEIGYLALHIGGAIERRKIINQPKRCLIVCASGVGSAKLLSYKLQATFGLNLTILGTTQYYKLHQMSLHSLDFIVSTIPIPDHLPVPVVEVSTILGNSDFKKIECLMKNKPQPSLEYTKKELVYLQQEFQTREEVLHFLGEKLTHLGLASSQFIESVFEREAASPTSFGNLVAIPHPMVPQTDTTFWAICTLKKPIMWEGNLVQFICLLSVKANNQGDLQKMYSLLVQVVDDKQVVQRLVKCKDYEEFIEVFMKNIR

SEQ ID NO: 86 - protein, 783 aa, Bacillus aryabhattai
MKSWKKILGTSFLATTVTLSGVGPVLMNGSTASAKTAQSDELPSSPIDMNTVPEERLANALKNQGVISQSATPQEVKKAVTSYINKKDAHKESMKGHKASKIDLKAKEMQTKQKEKFQRGEFANIKSQGKNSGNGVNVKPAAQANYKGQVRKDKVLVLLVEYADFKHNNVVQEPGYMYAKDFNRNHYQQLMFGKKDFTLFNGKKIKTFKQYYEEQSGGSYTVDGTVSDWLTVPGNAKEYGADNPAGGNDNAPGSKGPRNLVKEALAAAAKSGINLSDYDQFDQYDIDGDGNRNEPDGIVDHLMVIHAGTGQEAGGGKLGNDAIWSHRSTLGQVYKVPGTNMAAYDYTIEPEDGAVGVFAHEFGHDLGLPDEYDTQYTGQGEPVGVWSIMSGGSWAGEVAGTEPTSFSPQNKEFFQANMGGNWANIQQIDSKNITKSGSVSVVDQSVTKSKRPGIVKINLPKKAVQGIKPEFGSNYYYSKRGDDLHTTMTTPEFDLTTAKTATFNYKAFYEVEAEYDYLYVNAILPDGSKVLLDTIGDKANNADGSAETSGGNWEDKSYDLTQFKGKKIKLQFEYVTDGGLALQGFALDNASLVVDGKTVFTDDAEGAPKVTLDGFEKTNGISYKDNYYYLEWRNYAGSDKALQFARGAKYNTGMLLWYADESYLDNWVGQHPGEGFLGVVDSHAHKVLYFNVNGISTTANSSRYQIADAAFSFDKSPAWSYSHKTWGTISSKGSNGVTSFNDKKSYLDERIKDAGRLLPQNGLKLDVIGEAKDNSAGAVWIHK

SEQ ID NO: 87 - protein, 384 aa, Bacillus aryabhattai
MKLYALCKKTWAMSLLIFVLLVSLTPVSQAAAFLSVAQALENQNNSVQTVKGYVVGQPTGTSTVITSNYPNDYALALADSSNETNTDKMVYVQIPSNLRSTFGLQSHSELKGKSLTVTGTLTPYFSHSGIKSVTSISTETGGTDPAPDPTEPTVPVEDYYRTAAEKTGNSLKTELHNIIDHHTELSYSAVWEALKKTDEDPANANNVILLYTGRSQAKGTNGGGVDDWNREHVWAKSHGDFGTAMGPGTDLHHLRPTDVSVNSTRGNLDFDNGGTEHSEALGNYFDSDSWEPRDEVKGDVARMLFYMAVRYEGDVSDEPDLELNNTVNNGTAPYHGKLSVLLQWNAQDPVDDRERRRNDIIYSDYQHNRNPFIDHPEWVNEIWN

SEQ ID NO: 88 - protein, 434 aa, Bacillus aryabhattai
MSEKKPTYLEEQLEAVMTRHNNEYTFIFQKEKIRLNRSVEIEMLKDVNTAFQKEIMMSDDELKITIKPTSEFQPFTALRGKEEQQKWLFADQLIKQVKQHSFSRLHLLICPENIIFDKSLSPAFLHYGVKESLPPYESNEEQSFQELKATIAAAVDSSHTFEQYFKLYETLQLKEDAKKIMHISNEEELSQFVQHKLTQIEKHQKTLVQLPQKRWTIFKFSSVGILVLLIPALIYTFYSYFFAQPKQEAFVESNEYFLEKNYSKVVDTLEDYDIESMPKIVQYEIATSYLVNEKLGEEQKANALKTITLQSDPQYFAYWFYIGRGDNKKALEVSRLLEERSLIIYALMKYEGQLKTDNDLAADKKQEELDKVANELEEFKKENDQQEAEAAKQQEEKQAEEAQAAKQQEEDQKAAEAEKKKQEEQKKQQEDQSK

SEQ ID NO: 89 - protein, 1492 aa, Bacillus aryabhattai
MNMLWVVSGQTYQQLPVDFKTFRQATVGNTQHQTFTFSYPFEKGQMTLSSEKNGRLLISYGEERIAELSVSESFVWREQGHALTFVLTETAVKEAVYYIDYGSEVSFSTSDEAADVYQQKQKCVVEPKEGFYLSKINGKWKVFYNQEPLYLNGHRLEENTNLQPGALILWSSMTIQLIEKDLLKIQSYYDYASKLPATAQPISEMKKKYPVYRRTPRMIYDLPNDKVSLSFPSQDNDENNRSLWLTILPPLVMLLVMGTVSIIQPRGIFIIISVMMFTTTLIVSTVQYFKDKKQRKKRQERRRRIYTKYLENKREELQELVSVQRDVLSYHFPTFERMKYLTEQISDRIWERTVESSDFLQVRLGTGTVPGSYQISLNSGDMANREIDDLLEQSQHMEKVYREVKNAPVTAHLGLGSIGLVGKMKTVKNELHQLVGQLAFSHSYHDLRFVFIFDEEEYKEWEWMKWLPHFQLPHSFAKGFIYNEQTRDQLLSSIYEMLRERDLDEAKGEGVFLPHFVFIITNQQLIADHVILEYLEGYHPHLGFSAIFAAETKESLTEHVHTLVRYINESEGDILIQKQKAVNIPFQLDKHTLDYNERFARMLRTLDHQVGMTNSIPNSVSFLELFKAREIGELDIVQRWQSNESAKSLAVPIGLKGKEDIVELNLHEKAHGPHGLLAGTTGSGKSEFLQTYILSLAVNFHPHEVAFLLIDYKGGGMAQPFKNMPHLLGTITNIEGSKNFSTRALASIKSELKRRQRLFDRYEVNHINDYTDLYKQGMAEEPLPHLFLISDEFAELKSEEPEFIRELVSAARIGRSLGVHLILATQKPGGVIDDQIWSNARFKVALKVQDAADSKEILKNADAASITVTGRGYLQVGNNEVYELFQSAWSGAPYMEDTYGAEDEVAIVTDLGLIPLSDVSTQQTSSKQAEAEIDVVVQKIEKTQQELAIKKLNSPWLPPLKERLLRSEYKEKRDGLFPIGLIDEPEKQSQTVYNYQLMDDGNIGIFGSSGYGKSFTVMMLLLSLAERQSPEQLHYYIFDFGNGTLLPLRQLPHTADYFLMDQMRKIEKFMTIIKQEIARRKQLFQQREVSNIKMYNALSSEELPLIFITIDNFDLVKEEMQDLEMQFTQLVRDGQSLGIYMIFTATRVNSIRQSLMNNLKTKVVHYLMDHSEAYSILGRTPYALESIPGRAIIKTDQPYFAQLFLPAEGKDDFEIFDAVKLHIQRLKNQYADATVPEPVPMLSSKLVLAEFMTDRRVIREKGVIPLGLDEEFVRPVHMNFTKNKHCIVMGQTQKGKTNVVKVILQTALEQGVKQIGLFDSVDRGLSSYAAEEQVVYMETKEQIIDWLDLTEERLQRRENKYLEAVQEGTVHSLSFAPIMLVVDGFVRFQQNLDSVLQDRITKLMKNYSHLGFNLIVAGNNNEFSKGFDALTTEVKQISQAVLLMKKTEQSLFTLPYDRKEADVNPGFGYYIKSGKETRIQIPLCVSERKILT

SEQ ID NO: 90 - protein, 105 aa, Bacillus aryabhattai
MDAIDDLFDDIERRRKSKEYSRDADQLESYLHEVQRIMEFLEEGIYLFQNSHQQYASDWSGRSKSSYEDIYNDITQSTFHLYDVRDELFQTLRLEISRLRELASA

SEQ ID NO: 91 - protein, 199 aa, Bacillus aryabhattai
MEIKVNPEVLQTFAERIQQSHSQTADALQKLSWEATNLHFLSVADIDKVLDLQEELQHMIRKLDSLTENAHLLVKDTAEQMRKADGELEGSSGWTEFWSSVWSDGSTHSQDTWNDIKNLKEWNTYDNMHRTIEKPLGTLNVMWNSLSMSWEEKVVNGSTCSRTEFFTYGVIQMGLGILSQEMNPSNIMFSDSTEEVKRE

SEQ ID NO: 92 - protein, 130 aa, Bacillus aryabhattai
MKYTFNLLEKSLIEAMKDNKKIAQFYKTLMNSNLWLVVSSEHGASDILIAEHYVQTMKSAAYRYLPVFSSKYPIEQMLKDEKTVCVSFKDMLPVLNEEIAILLNPDTPLSKLFIPEEIRMLKEDQNYFKQ

SEQ ID NO: 93 - protein, 1057 aa, Bacillus aryabhattai
MTEQRKYTLKVILAVVLILALPSFFFQYIGDNPLQVTENSTRTIAVVNEDNGVKENSEKAIEFGEKVTPLLQEGSDYQWTVLGRSAATSGLQNGKYDAVIYIPSTFSQNILSYQEKNPEKATLEYKVQSQLNAVNKEKVVGELEDATTKVNNEMSSLYWSYVSQEVDKVRGQFDKILNKEIAFQKTMVAFYKPNSKDLAGELNDQKKILQQIQDNVNKAEKGSSDRQGDVEQVEKNLNSFIEYVNQYQTYQETQKQMLEKTQAQSVAAIQEELSSLESNQPSSKQNFNEQANQVFSGLSSIQQQLDQNQKAVGELGEQQADNVSEQEKALQDLNGQIVSSYKQQSEQTTLNQIESKLKPLRAQLETASSNEGGGGSTDPGEDGEAQPDPNQGGETPTDPGEDNGTQPDPNQGGETPTDPGEDDGTQPDSGQGEGTSEPIKVDLSQQTAKLDDISKGLEDLEQQLQGMVNEAPEAPEAPKTSEGTETPETSDTADASGTSNAVKSALEKVANLKTQVQAVKDELQNVTVQPEASGENTDALKQKIASLEADVKSLTAQKNKAEKDLEEATKSSSGQIAKLQEENERLKKENEKLKEDGKKPSKGSVDVDNLITMIKNKEDKILSSPNLSASRKEKLSNTFSGKIQSSSAEDLLKYYGDLSQYDLTLAQIGNTDIQNSVLNNDSIKGGLQQALAGTVSSQDARKQIQQNLSLTKEELAQFQSSVQDFADQYGQTVDAEQASIMEELSAIQEKANAMNQELQSSAGEDQALEKKEAPDVTGLMTLQQSMGQELKGMNELISSLGERQANVVTYTDELQKRVNEVQDKADTLNSKWAQNVDSTKLVRGQVYRLLNNTLVDGQNNDYVYHYLANPLQVSGEVPAEKVKEVPPVVILVIILISSLLIGYFSQYYRHAPMLVKGTLFGLLNLIVGLMVSIFSLNIYSLSGDRAMEWSIFTIVLLLASSTLVRIAFLLGSFAGWVTTVGLILFYITPLLNLSMPNFNYEDPVSKVYMSIQYDTQSLFGQALIVLLGMTAVLSVLPSIIRALKTTKEVENDETYSA

SEQ ID NO: 94 - protein, 165 aa, Bacillus aryabhattai
MMKPIVLNKKIGSIVLIFSLLCFPIAAAAEADDGNVEEIKPNVYKDDPINLDPESLLENKKEVGNIPEEIKDLTFEKEEDAEKESLKESLFANASAEDNTISAKAKEYNLFSSKNTSYKKERTEQTVSEKSSDLQWFYLLLIGVGVVILFAVLIPRISPQPSVKK

SEQ ID NO: 95 - protein, 79 aa, Bacillus aryabhattai
MYIEVTVDLKHYTGDRFDLRLSNYHSLKKVVEIVWQAKKITQSPKDGAWIRVSNKNLIFSGNETMINCGITTGDCIEIL

SEQ ID NO: 96 - protein, 97 aa, Bacillus aryabhattai
MSGIIRVTPAELRDMAGRYTNESGQVQELVSRLDTMKNQLQDMWEGASSEAFAAQYEELKPSFVEMSNLLTKIAKQLDDSANVLEDTDNQIASQIRG

SEQ ID NO: 97 - protein, 87 aa, Bacillus aryabhattai
MGVLQNIWPLLLMFVIFYFLLIRPQQKRQKNVQQMQSALKKGDKVVTIGGMHGLVDAIDESQVVVRSTDGSKLTFDRNAIREVRSVD

SEQ ID NO: 98 - protein, 21 aa, Bacillus aryabhattai
QNIWPLLLMFVIFYFLLIRPQ

SEQ ID NO: 99 - protein, 25 aa, Bacillus aryabhattai
MGVLQNIWPLLLMFVIFYFLLIRPQ

SEQ ID NO: 100 - protein, 25 aa, unidentified
MGVFQNIWPLLLMFVIFYFLLIRPQ

SEQ ID NO: 101 - protein, 25 aa, unidentified
MGVFQQIWPLLLMFVIFYFLLIRPQ

SEQ ID NO: 102 - protein, 25 aa, unidentified
MGVFNNIWPLLLMFVIFYFLLIRPQ

SEQ ID NO: 103 - protein, 25 aa, unidentified
MGVFNQIWPLLLMFVIFYFLLIRPQ

SEQ ID NO: 104 - protein, 25 aa, unidentified
MGVFNQIYPLLLMFVIFYFLLIRPQ

SEQ ID NO: 105 - protein, 25 aa, unidentified
MGVFNNIYPLLLMFVIFYFLLIRPQ

SEQ ID NO: 106 - protein, 25 aa, unidentified
MGVFQNIYPLLLMFVIFYFLLIRPQ

SEQ ID NO: 107 - protein, 25 aa, unidentified
MGVMQNIWPLLLMFVIFYFLLIRPQ

SEQ ID NO: 108 - protein, 25 aa, unidentified
MGVMQQIWPLLLMFVIFYFLLIRPQ

SEQ ID NO: 109 - protein, 25 aa, unidentified
MGVMNNIWPLLLMFVIFYFLLIRPQ

SEQ ID NO: 110 - protein, 25 aa, unidentified
MGVMNQIWPLLLMFVIFYFLLIRPQ

SEQ ID NO: 111 - protein, 25 aa, unidentified
MGVMNQIYPLLLMFVIFYFLLIRPQ

SEQ ID NO: 112 - protein, 25 aa, unidentified
MGVMNNIYPLLLMFVIFYFLLIRPQ

SEQ ID NO: 113 - protein, 25 aa, unidentified
MGVMQNIYPLLLMFVIFYFLLIRPQ

SEQ ID NO: 114 - protein, 25 aa, unidentified
MGVVQNIWPLLLMFVIFYFLLIRPQ

SEQ ID NO: 115 - protein, 25 aa, unidentified
MGVVQQIWPLLLMFVIFYFLLIRPQ

SEQ ID NO: 116 - protein, 25 aa, unidentified
MGVVNNIWPLLLMFVIFYFLLIRPQ

SEQ ID NO: 117 - protein, 25 aa, unidentified
MGVVNQIWPLLLMFVIFYFLLIRPQ

SEQ ID NO: 118 - protein, 25 aa, unidentified
MGVVNQIYPLLLMFVIFYFLLIRPQ

SEQ ID NO: 119 - protein, 25 aa, unidentified
MGVVNNIYPLLLMFVIFYFLLIRPQ

SEQ ID NO: 120 - protein, 25 aa, unidentified
MGVVQNIYPLLLMFVIFYFLLIRPQ

SEQ ID NO: 121 - protein, 25 aa, unidentified
MGVLQQIWPLLLMFVIFYFLLIRPQ

SEQ ID NO: 122 - protein, 25 aa, unidentified
MGVLQQIYPLLLMFVIFYFLLIRPQ

SEQ ID NO: 123 - protein, 25 aa, unidentified
MGVLNNIWPLLLMFVIFYFLLIRPQ

SEQ ID NO: 124 - protein, 25 aa, unidentified
MGVLNQIWPLLLMFVIFYFLLIRPQ

SEQ ID NO: 125 - protein, 25 aa, unidentified
MGVLNQIYPLLLMFVIFYFLLIRPQ

SEQ ID NO: 126 - protein, 25 aa, unidentified
MGVLNNIYPLLLMFVIFYFLLIRPQ

SEQ ID NO: 127 - protein, 25 aa, unidentified
MGVLQNIYPLLLMFVIFYFLLIRPQ

SEQ ID NO: 128 - protein, 21 aa, unidentified
NNIWPLLLMFVIFYFLLIRPQ

SEQ ID NO: 129 - protein, 21 aa, unidentified
NNIYPLLLMFVIFYFLLIRPQ

SEQ ID NO: 130 - protein, 21 aa, unidentified
NQIWPLLLMFVIFYFLLIRPQ

SEQ ID NO: 131 - protein, 21 aa, unidentified
NQIYPLLLMFVIFYFLLIRPQ

SEQ ID NO: 132 - protein, 21 aa, unidentified
QNIYPLLLMFVIFYFLLIRPQ

SEQ ID NO: 133 - protein, 21 aa, unidentified
QQIWPLLLMFVIFYFLLIRPQ

SEQ ID NO: 134 - protein, 21 aa, unidentified
QQIYPLLLMFVIFYFLLIRPQ